(19) United States
(12) Patent Application Publication
(10) Pub. No.: US 2026/0125467 A1
(43) Pub. Date: May 7, 2026

(54) NOVEL ANTI-LILRB4 ANTIBODIES AND DERIVATIVE PRODUCTS

(71) Applicant: IMMUNE-ONC THERAPEUTICS, INC., Palo Alto, CA (US)

(72) Inventors: An SONG, Palo Alto, CA (US); Tao HUANG, Mountain View, CA (US); Ryan STAFFORD, Foster City, CA (US); Maria Jose COSTA, Palo Alto, CA (US); Kyu Hee HONG, Palo Alto, CA (US); Caroline BONNANS, San Francisco, CA (US); Jianhui ZHOU, Redwood City, CA (US); Li ZHOU, San Jose, CA (US); Ji LI, Foster City, CA (US); J. Paul WOODARD, Palo Alto, CA (US); X. Charlene LIAO, Palo Alto, CA (US)

(21) Appl. No.: 19/369,309

(22) Filed: Oct. 26, 2025

Related U.S. Application Data
(63) Continuation of application No. 17/905,943, now Pat. No. 12,559,555.

(30) Foreign Application Priority Data
Mar. 12, 2020 (US) 62988892

(51) Int. Cl. C07K 16/28; A61K 38/17; A61K 39/00; A61K 39/395; A61K 40/11; A61K 40/24; A61K 40/31; A61K 40/42; A61K 45/06; A61P 35/02; C07K 14/705; C07K 14/725; C12N 5/0783

(52) U.S. Cl. CPC C07K 16/2803; A61K 38/177; A61K 38/1774; A61K 39/3955; A61K 40/11; A61K 40/24; A61K 40/31; A61K 40/4203; A61K 45/06; A61P 35/02; C07K 14/7051; C07K 14/70517; C07K 14/70521; C07K 14/70578; C07K 16/2809; C12N 5/0636; A61K 2039/505; A61K 2239/21; A61K 2239/22; A61K 2239/29; C07K 2317/24; C07K 2317/31; C07K 2317/622; C07K 2317/73; C07K 2317/732; C07K 2317/92; C07K 2319/02; C07K 2319/03; C07K 2319/33

(57) ABSTRACT

The present disclosure provides bispecific antibodies or antigen-binding fragments capable of binding to LILRB4 and CD3, isolated polynucleotides encoding the same, pharmaceutical compositions comprising the same, and the uses thereof.

### CROSS-REFERENCE TO RELATED APPLICATIONS

This application is a continuation application of U.S. application Ser. No. 17/905,943, which is the national phase of PCT/US2021/022029 filed on Mar. 12, 2021, which claims priority to U.S. provisional patent application No. 62/988,892, filed Mar. 12, 2020, the disclosure of which is incorporated herein by reference.

### SEQUENCE LISTING

The sequence listing that is contained in the file named “066564-8013WO01_ST25”, which is 150,794 bytes (as measured in Microsoft Windows) and was created on Mar. 12, 2021, is filed herewith by electronic submission and is incorporated by reference herein.

### FIELD OF THE INVENTION

The present disclosure relates generally to the fields of medicine, oncology, and immunology. More particular, the disclosure relates to antibodies that bind to LILRB4.

### BACKGROUND

Human Leukocyte Immunoglobulin-Like Receptor subfamily B member 4 (LILRB4), also known as Immunoglobulin-like transcript 3 (ILT3 or ILT-3), Leukocyte Immunoglobulin-like Receptor 5 (LIR5 or LIR-5), and CD85k or CD85K, is a type I membrane protein that contains cytoplasmic immunoreceptor tyrosine-based inhibition motif (ITIM) and involves in negative regulation of immune cell activation. LILRB4 is expressed on monocytes, macrophages and dendritic cells and can inhibit innate immunity in a cell-autonomous manner as well as suppress T cell activation through an indirect mechanism. LILRB4 is a specific marker for monocytic acute myeloid leukemia (AML) including refractory and relapsed disease. It has been shown that LILRB4 supports tumor cell infiltration into tissues and suppresses T cell activity via a signaling pathway that involves APOE, LILRB4, SHP-2, uPAR and ARG1 in AML cells (Deng M. et al., Nature (2018) 562:605-09). There is a significant need for novel anti-LILRB4 antibodies.

### BRIEF SUMMARY OF THE INVENTION

The present disclosure provides anti-LILRB4 antibodies and antigen-binding fragment thereof, amino acid and nucleotide sequences thereof, anti-LILRB4 chimeric antigen receptors, and uses thereof.

In one aspect, the present disclosure provides an isolated anti-LILRB4 antibody or an antigen-binding fragment thereof. In some embodiments, the anti-LILRB4 antibody or an antigen-binding fragment comprises: (a) a heavy chain variable region comprising a heavy chain complementarity determining region (HC-CDR) 1 having an amino acid sequence of SEQ ID NO: 5, an HC-CDR2 having an amino acid sequence of SEQ ID NO: 6 and an HC-CDR3 having an amino acid sequence of SEQ ID NO: 7; and (b) a light chain variable region comprising a light chain complementarity determining region (LC-CDR) 1 having an amino acid sequence of SEQ ID NO: 8 with a mutation at amino acid residues NS, an LC-CDR2 having an amino acid sequence of SEQ ID NO: 9 and an LC-CDR3 having an amino acid sequence of SEQ ID NO: 10.

In certain embodiments, the LC-CDR1 has an amino acid sequence of SEQ ID NO: 28.

In certain embodiments, the heavy chain variable region has an amino acid sequence at least about 90% identical to SEQ ID NO: 1; and wherein the light chain variable region has an amino acid sequence at least about 90% identical to SEQ ID NO: 27.

In certain embodiments, the heavy chain variable region has an amino acid sequence of SEQ ID NO: 1; and wherein the light chain variable region has an amino acid sequence of SEQ ID NO: 27.

In certain embodiments, the antibody or the antigen-binding fragment further comprises an immunoglobulin constant region, optionally a constant region of Ig, or optionally a constant region of human IgG.

In certain embodiments, the antibody described herein is of the IgG1, IgG2, IgG3 or IgG4 isotype.

In certain embodiments, the antibody or the antigen-binding fragment is humanized.

In certain embodiments, the antigen-binding fragment is a camelized single domain antibody, a diabody, a ds (disulfide-stabilized) diabody or ds diabody, a scFv, a scFv dimer, a BsFv, a dsFv, a (dsFv)₂, a dsFv-dsFv′, an Fv fragment, a Fab, a Fab′, a F(ab′)₂, a bispecific antibody, a nanobody, a domain antibody, or a bivalent antibody.

In certain embodiments, the anti-LILRB4 antibody described herein is a bispecific antibody. In some embodiments, the anti-LILRB4 bispecific antibody is against a T-cell receptor such as CD3. In some embodiments, the anti-LILRB4 bispecific antibody is against an NK-cell receptor such as CD16A.

Therefore, the present disclosure in another aspect provides a bispecific antibody or antigen-binding fragment capable of binding to LILRB4 and CD3.

In certain embodiments, the bispecific antibody or antigen-binding fragment provided herein comprises: (a) a first antigen-binding region comprising a first light chain variable (V_{L}) domain and a first heavy chain variable (V_{H}) domain; and (b) a second antigen-binding region comprising a second V_{L} domain and a second V_{H} domain, wherein the first antigen-binding region is capable of binding to LILRB4 and the second antigen-binding region is capable of binding to CD3, or vice versa.

In certain embodiments, the first V_{L} domain and the first heavy chain variable domain link to a first pair of constant domains, respectively, and wherein the second V_{L} domain and the second V_{H} domain link to a second pair of constant domains, respectively.

In certain embodiments, the first V_{L} domain links to a first light chain constant (C_{L}) domain, and the first V_{H} domain links to a first heavy chain constant domain 1 (C_{H}1). In certain embodiments, the first V_{L} domain links to a first C_{H}I domain, and the V_{H} domain links to a second C_{L} domain.

In certain embodiments, the second V_{L} domain links to a second C_{L} domain, and the second V_{H} domain links to a second C_{H}1 domain. In certain embodiments, the second V_{L} domain links to a second C_{H}1 domain, and the second V_{H} domain links to a second C_{L} domain. In certain embodiments, the second V_{L} domain links to a T cell receptor (TCR) a chain constant domain, and the second V_{H} domain links to a TCR β chain constant domain. In certain embodiments, the second V_{L} domain links to a TCR β chain constant domain, and the second V_{H} domain links to a TCR α chain constant domain.

In certain embodiments, the first antigen-binding region and/or the second antigen-binding region is a single chain variable fragment (scFv).

In certain embodiments, the antibody or antigen-binding fragment provided herein further comprises a third antigen-binding region comprising a third V_{L} domain and a third V_{H} domain, wherein the third antigen-binding region is capable of binding to LILRB4 or CD3.

In certain embodiments, the third V_{L} domain and the third heavy chain variable domain link to a first pair of constant domains, respectively. In certain embodiments, the third V_{L} domain links to a third C_{L} domain, and the third V_{H} domain links to a third C_{H}1 domain. In certain embodiments, the third V_{L} domain links to a third C_{H}1 domain, and the third V_{H} domain links to a third C_{L} domain. In certain embodiments, the third V_{L} domain links to a second TCR α chain constant domain, and the third V_{H} domain links to a second TCR β chain constant domain. In certain embodiments, the third V_{L} domain links to a second TCR β chain constant domain, and the third V_{H} domain links to a second TCR α chain constant domain.

In certain embodiments, the TCR α chain constant domain has an amino acid sequence of SEQ ID NO: 89. In certain embodiments, the TCR α chain constant domain has a S91A mutation of SEQ ID NO: 89.

In certain embodiments, the antibody or the antigen-binding fragment is linked to one or more conjugate moieties. In certain embodiments, the conjugate moiety comprises a clearance-modifying agent, a toxin, a detectable label, a chemotherapeutic agent, or purification moiety.

In another aspect, the present disclosure provides a pharmaceutical composition comprising the antibody or antigen-binding fragment thereof described herein, and a pharmaceutically acceptable carrier.

In another aspect, the present disclosure provides an isolated polynucleotide encoding the antibody or antigen-binding fragment thereof described herein.

In another aspect, the present disclosure provides a vector comprising the isolated polynucleotide described herein.

In another aspect, the present disclosure provides a host cell comprising the vector described herein. In certain embodiments, the host cell is a mammalian cell, e.g., a CHO cell.

In another aspect, the present disclosure provides a hybridoma encoding or producing the anti-LILRB4 antibody as provided herein.

In another aspect, the present disclosure provides a method of expressing the antibody or antigen-binding fragment thereof described herein. In some embodiments, the method comprises culturing the host cell described herein under the condition at which the vector described herein is expressed.

In another aspect, the present disclosure provides a method of treating or ameliorating the effect of a cancer in a subject. In some embodiments, the method comprises administering to the subject a therapeutically effective amount of the antibody or antigen-binding fragment thereof described herein or the pharmaceutical composition described herein.

The method may reduce or eradicate the tumor burden in the subject, may reduce the number of tumor cells, may reduce tumor size, may reduce tumor infiltration, may reduce tumor metastasis, may eradicate the tumor in the subject. The cancer may be a solid tumor or hematologic malignancy.

In certain embodiments, the cancer is adrenal cancer, bile duct carcinoma, bone cancer, brain cancer, breast cancer, cervical cancer, choriocarcinoma, colon cancer, colorectal cancer, esophageal cancer, eye cancer, gastric cancer, gastroesophageal cancer, glioblastoma, head and neck cancer, kidney cancer, liver cancer, lung cancer, non-small cell lung cancer, bronchioloalveolar cell lung cancer, mesothelioma, squamous cell carcinoma, melanoma, merkel cell cancer, nasopharyngeal carcinoma, neuroblastoma, oral cancer, ovarian cancer, pancreatic cancer, penile cancer, prostate cancer, renal cell cancer, retinoblastoma, sarcoma, skin cancer, testicular cancer, thymic carcinoma, thyroid cancer, uterine cancer, and vaginal cancer.

In some embodiments, the cancer is a metastatic, recurrent or drug-resistant cancer.

In some embodiments, said cancers are hematologic malignancies including acute lymphocytic/lymphoblastic leukemia (ALL), acute myeloid leukemia (AML), B-cell leukemia, blastic plasmacytoid dendritic cell neoplasm (BPDCN), chronic lymphoblastic leukemia (CLL), chronic myelomonocytic leukemia (CMML), chronic myelocytic leukemia (CML), diffuse large B-cell lymphoma (DLBCL), extranodal NK/T-cell lymphoma, hairy cell leukemia, HHV8-associated primary effusion lymphoma, plasmablastic lymphoma, pre-B acute lymphocytic leukemia (Pre-B ALL), primary CNS lymphoma, primary mediastinal large B-cell lymphoma, T-cell/histiocyte-rich B-cell lymphoma, heavy chain disease, Hodgkin's lymphoma, non-Hodgkin's lymphoma, Waldenstrom's macroglobulinemia, multiple myeloma (MM), myelodysplastic syndromes (MDS), myeloproliferative neoplasms, and polycythemia vera.

In certain embodiments, said hematologic malignancies include subsets or subtypes of acute myeloid leukemia (AML), acute promyelocytic leukemia (APL) or M3 AML, acute myelomonocytic leukemia or M4 AML, acute monocytic/monoblastic leukemia or M5 AML, and acute myeloblastic leukemia.

In certain embodiments, said hematologic malignancies include acute myeloid leukemia (AML) that is resistant to venetoclax, or venetoclax in combination with azacytidine/azacitidine, that is relapsed after treatment with azacytidine/azacitidine and/or venetoclax, that is resistant to venetoclax in combination with decitabine or is relapsed after treatment with azacytidine/azacitidine and decitabine.

In certain embodiments, the antibody or an antigen-binding fragment thereof is administered intravenously, intra-arterially, intra-tumorally, or subcutaneously.

In certain embodiments, the method further comprises administering to the subject one or more drugs selected from the group consisting of a topoisomerase inhibitor, an anthracycline topoisomerase inhibitor, an anthracycline, a daunorubicin, a nucleoside metabolic inhibitor, a cytarabine, a hypomethylating agent, a low dose cytarabine (LDAC), a combination of daunorubicin and cytarabine, a daunorubicin and cytarabine liposome for injection, Vyxeos®, an azacytidine, Vidaza®, a decitabine, an all-trans-retinoic acid (ATRA), an arsenic, an arsenic trioxide, a histamine dihydrochloride, Ceplene®, an interleukin-2, an aldesleukin, Proleukin®, a gemtuzumab ozogamicin, Mylotarg®, an FLT-3 inhibitor, a midostaurin, Rydapt®, a clofarabine, a farnesyl transferase inhibitor, a decitabine, an IDH1 inhibitor, an ivosidenib, Tibsovo®, an IDH2 inhibitor, an enasidenib, Idhifa®, a smoothened (SMO) inhibitor, a glasdegib, an arginase inhibitor, an IDO inhibitor, an epacadostat, a BCL-2 inhibitor, a venetoclax, Venclexta®, a platinum complex derivative, oxaliplatin, a kinase inhibitor, a tyrosine kinase inhibitor, a PI3 kinase inhibitor, a BTK inhibitor, an ibrutinib, IMBRUVICA®, an acalabrutinib, CALQUENCE®, a zanubrutinib, a PD-1 antibody, a PD-L1 antibody, a CTLA-4 antibody, a LAG3 antibody, an ICOS antibody, a TIGIT antibody, a TIM3 antibody, a CD40 antibody, a 4-1BB antibody, a CD47 antibody, a SIRP1α antibody or fusions protein, a CD70 antibody, and CLL1 antibody, a CD123 antibody, an antagonist of E-selectin, an antibody binding to a tumor antigen, an antibody binding to a T-cell surface marker, an antibody binding to a myeloid cell or NK cell surface marker, an alkylating agent, a nitrosourea agent, an antimetabolite, an antitumor antibiotic, an alkaloid derived from a plant, a hormone therapy medicine, a hormone antagonist, an aromatase inhibitor, and a P-glycoprotein inhibitor.

In certain embodiments, the method further comprises administering to the subject initially a monotherapy of anti-LILRB4 antibodies for a period of time followed by addition of one or more drugs selected from the group consisting of an azacytidine, Vidaza®, a BCL-2 inhibitor, a venetoclax, and Venclexta®.

In yet another aspect, the present disclosure provides a method for detecting a cancer cell or cancer stem cell in a sample or subject. In certain embodiments, the method comprises: (a) contacting a subject or a sample from the subject with the antibody or an antigen-binding fragment thereof described herein; and (b) detecting binding of said antibody to a cancer cell or cancer stem cell in said subject or sample.

In some embodiments, the sample is a body fluid or biopsy. In some embodiments, the sample is blood, sputum, tears, saliva, mucous, serum, urine or feces.

In some embodiments, the detection comprises immunohistochemistry, flow cytometry, immunoassays (including ELISA, RIA etc.) or Western blot.

In some embodiments, the method further comprises performing steps (a) and (b) a second time or additional times and determining a change in detection levels as compared to the first time.

The anti-LILRB4 antibody or an antigen binding fragment thereof may further comprise a label, such as a peptide tag, an enzyme, a magnetic particle, a chromophore, a fluorescent molecule, a chemo-luminescent molecule, or a dye. The isolated monoclonal antibody or an antigen binding fragment thereof may be conjugated to a liposome or nanoparticle.

In another aspect, the present disclosure provides use of the antibody or antigen-binding fragment thereof described herein in the manufacture of a medicament for treating cancer in a subject.

In another aspect, the present disclosure provides a kit comprising the antibody or antigen-binding fragment thereof described herein, useful in detecting LILRB4.

In another aspect, the present disclosure provides an anti-LILRB4 chimeric antigen receptor (CAR) protein. In some embodiments, the CAR protein comprises: (a) a heavy chain variable region comprising an HC-CDR1 having an amino acid sequence of SEQ ID NO: 5, an HC-CDR2 having an amino acid sequence of SEQ ID NO: 6 and an HC-CDR3 having an amino acid sequence of SEQ ID NO: 7; and (b) a light chain variable region comprising an LC-CDR1 having an amino acid sequence of SEQ ID NO: 8 with a mutation at amino acid residues NS, an LC-CDR2 having an amino acid sequence of SEQ ID NO: 9 and an LC-CDR3 having an amino acid sequence of SEQ ID NO: 10. In some embodiments, the LC-CDR1 has an amino acid sequence of SEQ ID NO: 28.

In some embodiments, the heavy chain variable region of the LILRB4 CAR protein has an amino acid sequence at least about 90% identical to SEQ ID NO: 1; and the light chain variable region of the CAR protein has an amino acid sequence at least about 90% identical to SEQ ID NO: 27. In some embodiments, the heavy chain variable region has an amino acid sequence of SEQ ID NO: 1; and wherein the light chain variable region has an amino acid sequence of SEQ ID NO: 27. In some embodiments, the CAR protein comprises a single-chain variable fragment (scFv) having an amino acid sequence at least 85%, 90%, 95% or 99% identical to SEQ ID NO: 66 or SEQ ID NO: 68. In some embodiments, the CAR protein has a scFv having an amino acid sequence identical to SEQ ID NO: 66 or SEQ ID NO: 68.

In another aspect, the present disclosure provides a polynucleotide molecule encoding a CAR protein described herein. In some embodiments, the polynucleotide molecule further comprises a promoter active in eukaryotic cells. In some embodiments, the polynucleotide molecule is an expression vector.

In another aspect, the present disclosure provides an engineered cell comprising the polynucleotide molecule encoding a CAR protein described herein. In some embodiments, the cell is a T cell, an NK cell or a macrophage.

In another aspect, the present disclosure provides a method of treating or ameliorating cancer in a subject in need thereof comprising administering to the subject an effective amount of a cell therapy comprising one or more cells comprising the polynucleotide molecule encoding a CAR protein described herein. In some embodiments, the method further comprises administering to said human subject a second cancer therapy. In some embodiments, the second cancer therapy is chemotherapy, immunotherapy, radiotherapy, hormone therapy or surgery. In some embodiments, the second cancer therapy is administered at the same time as the cell therapy. In some embodiments, said second cancer therapy is administered before or after the cell therapy. In some embodiments, the method further comprises administering to said human subject a second administration of an effective amount of one or more cells comprising the polynucleotide molecule encoding a CAR protein described herein.

In some embodiments, said cell therapy is administered local to cancer site, regional to a cancer site, or systemically.

In some embodiments, said cancer is hematologic malignancies including acute lymphocytic/lymphoblastic leukemia (ALL), acute myeloid leukemia (AML), B-cell leukemia, blastic plasmacytoid dendritic cell neoplasm (BPDCN), chronic lymphoblastic leukemia (CLL), chronic myelomonocytic leukemia (CMML), chronic myelocytic leukemia (CML), diffuse large B-cell lymphoma (DLBCL), extranodal NK/T-cell lymphoma, follicular lymphoma, hairy cell leukemia, HHV8-associated primary effusion lymphoma, plasmablastic lymphoma, pre-B acute lymphocytic leukemia (Pre-B ALL), primary CNS lymphoma, primary mediastinal large B-cell lymphoma, T-cell/histiocyte-rich B-cell lymphoma, heavy chain disease, Hodgkin's lymphoma, non-Hodgkin's lymphoma, Waldenstrom's macroglobulinemia, multiple myeloma (MM), myelodysplastic syndromes (MDS), myeloproliferative neoplasms, and polycythemia vera.

In certain embodiments, said hematologic malignancies include subsets or subtypes of acute myeloid leukemia (AML), acute promyelocytic leukemia (APL) or M3 AML, acute myelomonocytic leukemia or M4 AML, acute monocytic/monoblastic leukemia or M5 AML, and acute myeloblastic leukemia.

In still an additional aspect, there is provided a method of treating or ameliorating the effect of an autoimmune disease in a subject, the method comprising administering to the subject a therapeutically effective amount of the antibody or an antigen-binding fragment thereof as defined herein. The antibody or an antigen-binding fragment thereof may be administered intravenously, intra-arterially, intraperitoneally, or subcutaneously. The method may further comprise administering to the subject one or more drugs selected from the group consisting of a steroid or an NSAID. The autoimmune disease may be Guillain-Barre syndrome, Chronic inflammatory demyelinating polyneuropathy, ankylosing spondylitis, psoriatic arthritis, enteropathic arthritis, reactive arthritis, undifferentiated spondyloarthropathy, juvenile spondyloarthropathy, Behcet's disease, enthesitis, ulcerative colitis, Crohn's disease, irritable bowel syndrome, inflammatory bowel disease, fibromyalgia, chronic fatigue syndrome, pain conditions associated with systemic inflammatory disease, systemic lupus erythematosus, Sjogren's syndrome, rheumatoid arthritis, juvenile rheumatoid arthritis, juvenile onset diabetes mellitus (also known as Type I diabetes mellitus), Wegener's granulomatosis, polymyositis, dermatomyositis, inclusion body myositis, multiple endocrine failure, Schmidt's syndrome, autoimmune uveitis, Addison's disease, Grave's Disease, Hashimoto's thyroiditis, autoimmune thyroid disease, pernicious anemia, gastric atrophy, chronic hepatitis, lupoid hepatitis, atherosclerosis, multiple sclerosis, amyotrophic lateral sclerosis, hypoparathyroidism, Dressler's syndrome, myasthenia gravis, Eaton-Lambert syndrome, autoimmune thrombocytopenia, idiopathic thrombocytopenic purpura, hemolytic anemia, pemphigus vulgaris, pemphigus, dermatitis herpetiformis, alopecia, scleroderma, progressive systemic sclerosis, CREST syndrome (calcinosis, Raynaud's phenomenon, esophageal dysmotility, sclerodactyly, and telangtasia), adult onset diabetes mellitus (also known as Type II diabetes mellitus), mixed connective tissue disease, polyarteritis nodosa, systemic necrotizing vasculitis, glomerulonephritis, atopic dermatitis, atopic rhinitis, Goodpasture's syndrome, Chagas' disease, sarcoidosis, rheumatic fever, asthma, anti-phospholipidsyndrome, erythema multiforme, Cushing's syndrome, autoimmune chronic active hepatitis, allergic disease, allergic encephalomyelitis, transfusion reaction, leprosy, malaria, leshmaniasis, trypanosomiasis, Takayasu's arteritis, polymyalgia rheumatica, temporal arteritis, shistosomiasis, giant cell arteritis, eczema, lymphomatoid granulomatosis, Kawasaki's disease, endophthalmitis, psoriasis, erythroblastosis fetalis, eosinophilic faciitis, Shulman's syndrome, Felty's syndrome, Fuch's cyclitis, IgA nephropathy, Henoch-Schonlein purpura, graft versus host disease, transplantation rejection, tularemia, periodic fever syndromes, pyogenic arthritis, Familial Mediterranean Fever, TNF-receptor associated periodic syndrome (TRAPS), Muckle-Wells syndrome, or hyper-IgD syndrome.

### BRIEF DESCRIPTION OF FIGURES

FIG. 1 shows the schematics of the mechanisms of action of anti-LILRB4 antibodies and derivative products.

FIG. 2 shows the comparison of icIEF results of H7K3 (Molecule A) and H7K3m5 (Molecule B) at 40° C. for 2 W or 4 W.

FIG. 3 shows recognition of human endogenous LILRB4 on THP-1 cells by the anti-LILRB4 antibody H7K3m5.

FIG. 4 shows ADCC of THP-1-GFP cells by wild type (WT) or afucosylated (afu) H7K3m5.

FIG. 5 shows LILRB4 expression on human monocytes and plasmacytoid dendritic cells (pDC).

FIG. 6 shows up-regulation of LILRB4 expression by IL-10 and IFNα treatment in human monocytes.

FIG. 7 shows LILRB4 level down-regulated and uPAR level up-regulated on human monocytes stimulated by LPS.

FIG. 8A shows LILRB4 expression on in vitro human monocytes differentiated macrophages. FIG. 8B shows copy number of LILRB4 on in vitro monocyte-derived human macrophages.

FIG. 9 shows copy number of LILRB4 on in vitro differentiated MDSCs.

FIGS. 10A-10B show copy number of LILRB4 on human monocyte-derived dendritic cells (DCs). LILRB4 level is seen in the following order from high to low: Tolerogenic DC>Activated DC>Immature DC

FIG. 11 shows the comparison of LILRB4 mRNA expression levels between solid tumor samples from the TCGA RNA sequencing database with high and low signals for macrophage infiltration, based on a macrophage gene expression “signature” genera rated through computational biology approaches. The total number of samples for each tumor type that was included in the analysis is indicated in parentheses. The results shown here are in part based upon RNA sequencing data generated by the TCGA Research Network.

FIGS. 12A-12B illustrate the representative flow cytometry data showing that H7K3m5 specifically binds monocytic myeloid cells infiltrated into solid tumor microenvironment (TME), as well as peripheral blood monocytic myeloid cells from solid tumor patients. Dark grey-filled histogram: sample incubated with H7K3m5; light grey-filled histogram: sample incubated with human IgG1 isotype control. FIG. 12A. Binding signal of H7K3m5 or its isotype control on various myeloid cell subsets infiltrating the TME. FIG. 12B. Binding signal of H7K3m5 or its isotype control on various myeloid cell subsets from peripheral blood.

FIGS. 13A-13D show no H7K3m5 mediated monocyte killing in autologous ADCC using fresh PBMCs. Freshly isolated PBMCs from healthy donors were incubated overnight in the presence of serially titrated H7K3m5, isotype control human IgG1, or rituximab as positive control. Monocytes and B cells were identified and counted as CD14⁺CD19⁻ and CD19⁺CD14⁻ by flow cytometry. FIGS. 13A and 13C show lack of monocyte killing by PBMCs from two different donors and FIGS. 13B and 13D show corresponding B cell killing as positive control.

FIGS. 14A-14D show autologous ADCC of normal monocytes by wild type (WT) or afucosylated (afu) H7K3m5. FIGS. 14A and 14C show representative monocyte killing through ADCC by PBMCs from two different donors. ADCC on monocyte by afucosylated H7K3m5 was observed in donor 024 but activity was minimal in donor 13. No ADCC was observed by wild type H7K3m5. FIGS. 14B and 14D show corresponding B cell killing as positive control.

FIGS. 15A-15D show autologous ADCC of pDC or monocytes by wild type (WT) or afucosylated (afu) H7K3m5-mediated autologous ADCC. ADCC against pDCs was observed by both wild type and afucosylated H7K3m5 in two donors. In the meantime, monocytes may be killed only with afucosylated H7K3m5, depending on donors. Moreover, the afucosylated H7K3m5 showed much stronger ADCC activity than the wild-type towards pDC or monocytes (FIGS. 15A-15B).

FIGS. 16A-16B show dose-dependent ADCC of CD33+ MDSC cells with purified NK cells in the presence of H7K3m5, which has no ADCC effect on monocytes at the same dose levels. H7K3m5 showed ADCC activity against the AML cell line THP-1 in the same experiment (FIG. 16B).

FIGS. 17A-17B show ADCP of THP-1-GFP cells by anti-LILRB4. THP-1-GFP cells were co-cultured with in-vitro differentiated macrophages for 24 hours in the presence of serially titrated wild type H7K3m5 or isotype control human IgG1. THP-1-GFP cells and macrophages were identified and quantified as GFP+ and CD163+CD206+, respectively. Percent of THP-1 cell killing were calculated from absolute count of GFP+ cells or from GFP+% cells. FIGS. 17A and 17B show ADCP of THP-1-GFP cells by wild type H7K3m5 from macrophages differentiated from two separate heathy donors.

FIG. 18 shows in vitro T cell cytotoxicity against THP-1-GFP cells with anti-LILRB4. THP-1-GFP cells were co-cultured with purified naïve T cells. Anti-LILRB4 H7K3m5 can induce T-cell cytotoxicity against AML cells. Effector pan T cells were from 3 different healthy donors. The curves are plotted as mean±SD. The EC50 values are in nano molar units.

FIG. 19 shows representative cytokine production profile in the supernatant from the in vitro T-cell cytotoxicity assay samples. Anti-LILRB4 induced T-cell cytotoxicity against THP-1 cells is reflected by the elevation of cytokines in the co-culture. The curves are plotted as mean±SD. The EC₅₀ values are in nano molar units. An EC50 value cannot be derived for IL-6.

FIG. 20 shows the evaluation of T-cell activation by flow cytometry. A-B: surface staining of T cell activation markers CD69 (A) and CD25 (B). C-E: Intracellular cytokine staining of co-cultured T cells and THP-1 cells by flow cytometry. C: Cells producing both IFNγ and TNFα; D: Cells producing IFNγ but not TNFα; E: Cells producing TNFα but not IFNγ.

FIG. 21 shows increased T cell activation markers and MHC expression on THP-1 cells upon H7K3m5 treatment. D428=donor 428. MFI=geometric mean fluorescence intensity.

FIG. 22 shows surface expression of activation markers on THP-1 AML cells by flow cytometry. MFI=geometric mean fluorescence intensity.

FIG. 23 shows H7K3m5 is efficacious in AML xenograft model. This experiment evaluated the growth kinetics of THP-1.luc cells and determined the efficacy of H7K3m5 in the THP-1.luc human AML xenograft model in female NSG mice using bio-imaging. 1×10⁶ THP-1.luc cells were intravenously implanted to JAX female NSG mice via tail vein. Whole body bioluminescent imaging was conducted on Day 1 (4-6 hrs after cell injection) prior to animal randomization and single-dose intravenous administration of the vehicle control or H7K3m5 (1 mg/kg). On Days 7, 14, 17 and 21, whole body bioluminescent imaging data were collected for the control and treated animals.

FIG. 24 illustrates the flow cytometric data showing that H7K3m5 potentiates maturation/activation of monocyte-derived dendritic cells (Mo-DC) in response to Toll-Like Receptor (TLR) signaling. H7K3m5 enhanced the expression of activation markers (CD86, HLA-DR) while decreasing the expression of the tolerogenic marker CD209. Each line represents result from a different healthy donor. The fraction of donors in which H7K3m5 produced the desired pro-inflammatory effect is indicated in parentheses. *p<0.05 (paired t test).

FIG. 25 illustrates the flow cytometry data showing that H7K3m5 enhances the expression of activation markers (CD86 and HLA-DR) on the surface of mature monocyte-derived DC (Mo-DC) upon a mixed leukocyte reaction with allogeneic T cells. The effect of H7K3m5 was evaluated in the absence (−CD40L) or presence (+CD40L) of CD40 ligand to study the effect of H7K3m5 on immature and mature Mo-DC, respectively. Each line represents the result from a different healthy donor (n=3 donors).

FIG. 26 illustrates the ELISA data showing that H7K3m5 enhances IL-12 production in an allogeneic mixed leukocyte reaction of T cells and mature monocyte-derived DC (Mo-DC). The effect of H7K3m5 was evaluated in the absence (−CD40L) or presence (+CD40L) of CD40 ligand to study the effect of H7K3m5 on immature and mature Mo-DC, respectively. Data are presented as mean±SEM and data for each donor is also shown as individual data points (n=2-3 donors).

FIG. 27 illustrates the ELISA data showing that H7K3m5 enhances IFN-7 production in an allogeneic mixed leukocyte reaction of T cells and monocyte-derived DC (Mo-DC). The effect of H7K3m5 was evaluated in the absence (−CD40L) or presence (+CD40L) of CD40 ligand to study the effect of H7K3m5 on immature and mature Mo-DC, respectively. Data are presented as mean±SEM and data for each donor is also shown as individual data points (n=3 donors).

FIGS. 28A and 28B show the schematic representation of configurations of LILRB4/CD3 bispecific antibodies.

FIGS. 29A and 29B show the binding to normal monocytes and AML cell line THP-1 by CD3 and LILRB4 bispecific antibodies as measured by FACS. Similar binding affinity trends were observed across different anti-LILRB4 mono-specific and bispecific antibodies between monocytes and THP-1.

FIGS. 30A and 30B show T cell-mediated cytotoxicity of bispecific CD3/LILRB4 antibodies on monocytes (FIG. 22A) and THP-1-luc-GFP cells (FIG. 22B).

FIGS. 31A and 31B show the autologous killing of monocytes by LILRB4×CD3 bi-specifics (FIG. 31A) and autologous killing of B cells by Rituxan as a control (FIG. 31B).

FIG. 32 shows the binding affinity comparison using H7K3m5 full-length IgG and ScFv proteins in flow cytometry assays against human primary monocytes and human leukemia cell line THP-1.

FIG. 33 shows the schematic representation of the DNA construct for expressing the anti-LILRB4 CAR proteins. The DNA construct was based on 2^{nd} generation CAR constructs containing CD28 or 4-1BB costimulatory domain with CD3zeta activation domain. The scFv was derived from anti-LILRB4 monoclonal antibody H7K3m5. The 5′ and 3′ homologous arms are homologous sequences upstream and downstream of the Cas9 DNA cleavage site in the TRAC gene (based on gRNA design). Promoter and leader peptide are elements for gene expression and extracellular translocation. SV40 poly-A tail was included for improving transcript stability and translation.

FIG. 34 shows the efficient generation of LILRB4 CAR-T cells using CRISPR knock-out and knock-in method. Human primary T cells were transfected with CRISPR-Cas9 RNP complexes designed to inactivate the TCR alpha (TRAC) locus, with or without DNA template for homologous recombination-based knock in. Following transfection, cells were expanded in culture for 2 weeks. Anti-LILRB4 CAR-T cells were identified by binding to LILRB4-Fc fusion protein (ACRObiosystems CDK-H5259) and anti-Fc antibody (Biolegend B278652, negative control). Successful TCR alpha (TRAC) inactivation (Knock-Out or KO) was measured by anti-CD3 staining (anti-CD3 PE, BD 555333). ATC, activated T cells; KO, TCR alpha (TRAC) inactivated T cells; RB4_CD28, T cells expressing anti-LILRB4 CAR with a CD28 costimulatory domain; RB4_41BB, T cells expressing anti-LILRB4 CAR with a 4-1BB costimulatory domain.

FIG. 35 shows the proliferation of the TCR alpha (TRAC) inactivated T cells (KO) and anti-LILRB4 CAR (or control CAR) knocked-in T cells. After knocking out TCRalpha, the cells were grown in complete Optimizer medium with IL-2 300 IU/ml and without anti-CD3/28 added. Fold expansion was plotted by dividing the total T cell number on days (as indicated) with the starting culture number. Anti-LILRB4 CAR-T cells had significantly higher fold of expansion in comparison to the control CAR-T cells. ATC, activated T cells; KO, TCR alpha (TRAC) inactivated T cells; ctrl_CD28, T cells expressing a control CAR with a CD28 costimulatory domain; ctrl_41BB, T cells expressing a control CAR with a 4-1BB costimulatory domain; RB4_CD28, T cells expressing Anti-LILRB4 CAR with a CD28 costimulatory domain; RB4_41BB, T cells expressing Anti-LILRB4 CAR with a 4-1BB costimulatory domain.

FIGS. 36A-36H show the antigen-dependent activation of CAR-T culture. 1 μg/ml recombinant control antigen or LILRB4 antigen were coated on 96 well plates overnight in PBS buffer. Plates were washed twice with PBS buffer. 1×10⁵ CAR-T cells in culture media (without any cytokine added) were added to each well and incubated for 72 hours. Cell culture supernatant was collected for cytokine release measurement by Luminex assay. ATC, activated T cells; KO, TCR alpha (TRAC) inactivated T cells; antiRB4_CD28CART, T cells expressing Anti-LILRB4 CAR with a CD28 costimulatory domain; antiRB4_41BB CART, T cells expressing anti-LILRB4 CAR with a 4-1BB costimulatory domain; control_CD28CART, T cells expressing a control CAR with a CD28 costimulatory domain; control_41BBCART, T cells expressing a control CAR with a 4-1BB costimulatory domain.

FIGS. 37A-37C show the characterization of CAR-T cells after 2 weeks expansion. Frozen CAR-T cells in liquid nitrogen storage were thawed and kept in culture for 2-3 days before flow cytometry analysis. Antibodies used were anti-CD8 APC Cy7 (BD561945), anti-PD1 PE (BD560908) and anti-TIM3 BV421 (BD565562). Anti-LILRB4 CAR-T cells were identified by binding to LILRB4-Fc fusion protein (ACRObiosystems CDK-H5259) and anti-Fc antibody (Biolegend B278652).

FIG. 38 shows the cytotoxicity of anti-LILRB4 CAR-T cells. CHO K1 RB4 cells were seeded at different density (6×10⁴, 2×10⁴ or 7×10³) for 12 hours, 1×10⁵ CAR-T cells were added and cytotoxicity were measured by removing the supernatant CAR-T cells and wash the plate 2 times with PBS. Total viable adherent CHO K1 RB4 cells were measured by Promega CTG2.0 luminescence kit. And the % cytotoxicity were calculated by dividing the Luminescent signal of each condition with the same E:T ratio activated T cell control.

FIGS. 39A-39B show the schematics of a phase 1 first-in-human clinical trial. FIG. 39A is a schematic of the “window” design for dose escalation. FIG. 39B is a schematic of anti-LILRB4 monotherapy. FIGS. 39C-39D are the schematics of potential combination studies of anti-LILRB4 antibody with azacytidine and/or venetoclax. Other potential combinations with anti-LILRB4 would follow the same or a similar schema. AZA, azacytidine; VEN, venetoclax; C1D1, Cycle 1 Day 1; Cycle 2 Day 1; DLT, dose limiting toxicity; MTD1, maximum tolerated dose of anti-LILRB4 monotherapy; MTD2, maximum tolerated dose of anti-LILRB4 in combination with azacytidine. Anti-LILRB4 is administered as monotherapy or in combination with other agents every 14 days until progression of disease or death.

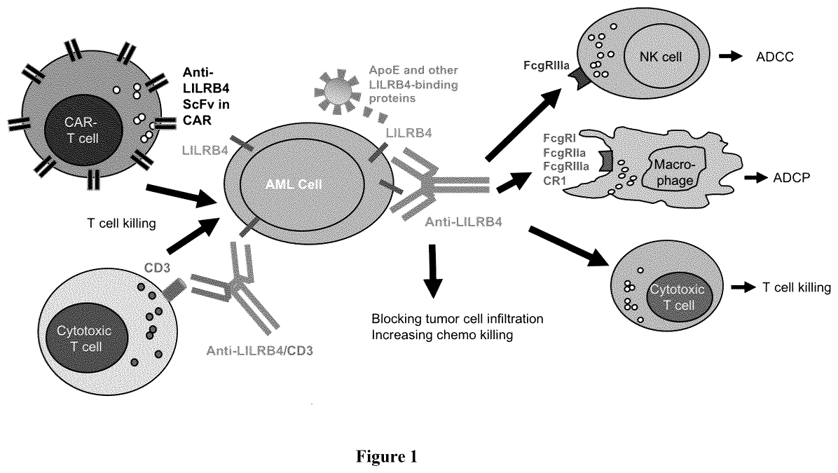

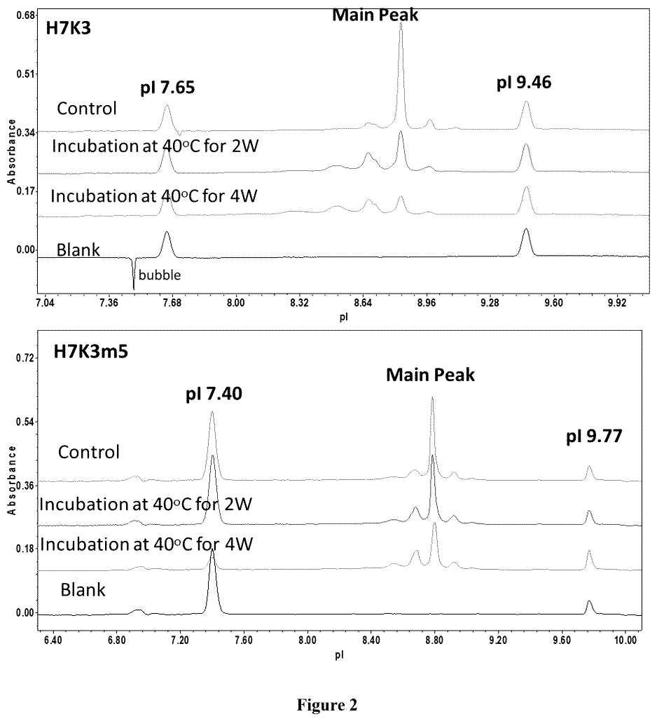

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### I. DEFINITIONS

It is to be understood that both the foregoing general description and the following detailed description are exemplary and explanatory only and are not restrictive of the invention as claimed. In this application, the use of the singular includes the plural unless specifically stated otherwise. In this disclosure, the term “or” is used to mean “and/or” unless explicitly indicated to refer to alternatives only or the alternatives are mutually exclusive. As used herein “another” may mean at least a second or more. Furthermore, the use of the term “including”, as well as other forms, such as “includes” and “included”, is not limiting. Also, terms such as “element” or “component” encompass both element or component comprising one unit and elements or components that comprise more than one subunit unless specifically stated otherwise. Also, the use of the term “portion” can include part of a moiety or the entire moiety.

As used herein, the singular forms “a”, “an” and “the” include plural references unless the context clearly dictates otherwise.

The term “antibody” as used herein includes any immunoglobulin, monoclonal antibody, polyclonal antibody, multivalent antibody, bivalent antibody, monovalent antibody, multi-specific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain consists of a variable domain (V_{H}) and a constant region including a first, second, and third constant domain (C_{H1}, C_{H2}, C_{H3}, respectively); mammalian light chains are classified as λ or κ, while each light chain consists of a variable domain (V_{L}) and a constant domain (C_{L}). A typical IgG antibody has a “Y” shape, with the stem of the Y typically consisting of the second and third constant domains of two heavy chains bound together via disulfide bonding. Each arm of the Y includes the variable domain and first constant domain of a single heavy chain bound to the variable and constant domains of a single light chain. The variable domains of the light and heavy chains are responsible for antigen binding. The variable domains in both chains generally contain three highly variable loops called the complementarity determining regions (CDRs) (light chain CDRs including LCDR1, LCDR2, and LCDR3, heavy chain CDRs including HCDR1, HCDR2, HCDR3). CDR boundaries for the antibodies and antigen-binding fragments disclosed herein may be defined or identified by the conventions of Kabat, IMGT, Chothia, or Al-Lazikani (Al-Lazikani, B., Chothia, C., Lesk, A. M., J. Mol. Biol., 273(4), 927 (1997); Chothia, C. et al., J Mol Biol. (1985) 186(3):651-63; Chothia, C. and Lesk, A. M., J.Mol.Biol. (1987) 196:901; Chothia, C. et al., Nature (1989) 342(6252):877-83; Marie-Paule Lefranc et al., Developmental and Comparative Immunology (2003) 27: 55-77; Marie-Paule Lefranc et al., Immunome Research (2005) 1(3); Marie-Paule Lefranc, Molecular Biology of B cells (second edition), chapter 26, 481-514, (2015)). The three CDRs are interposed between flanking stretches known as framework regions (FRs), which are more highly conserved than the CDRs and form a scaffold to support the hypervariable loops. The constant domains of the heavy and light chains are not involved in antigen-binding but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgAQ1 (alpha1 heavy chain), or IgA2 (alpha2 heavy chain).

The term “antigen” refers to a substance capable of inducing adaptive immune responses. Specifically, an antigen is a substance specifically bound by antibodies or T lymphocyte antigen receptors. Antigens are usually proteins and polysaccharides, less frequently also lipids. Suitable antigens include without limitation parts of bacteria (coats, capsules, cell walls, flagella, fimbrai, and toxins), viruses, and other microorganisms. Antigens also include tumor antigens, e.g., antigens generated by mutations in tumors. As used herein, antigens also include immunogens and haptens.

The term “antigen-binding fragment” as used herein refers to an antibody fragment formed from a portion of an antibody comprising one or more CDRs, or any other antibody fragment that binds to an antigen but does not comprise an intact native antibody structure. Examples of antigen-binding fragment include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a bispecific antibody, a multispecific antibody, a camelized single domain antibody, a nanobody, a domain antibody, and a bivalent domain antibody. An antigen-binding fragment is capable of binding to the same antigen to which the parent antibody binds.

A “Fab fragment” comprises one light chain and the C_{H}1 and variable domains of one heavy chain. The heavy chain of a Fab molecule cannot form a disulfide bond with another heavy chain molecule.

A “Fab′ fragment” comprises one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form an F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

“Fv” with regard to an antibody refers to the smallest fragment of the antibody to bear the complete antigen-binding site. An Fv fragment consists of the variable domain of a single light chain bound to the variable domain of a single heavy chain.

“Single-chain Fv antibody” or “scFv” refers to an engineered antibody consisting of a light chain variable domain and a heavy chain variable domain connected to one another directly or via a peptide linker sequence (Huston J S et al., Proc Natl Acad Sci USA (1988) 85:5879).

An “Fc” region comprises two heavy chain fragments comprising the C_{H}2 and C_{H}3 domains of an antibody. The two heavy chain fragments are held together by two or more disulfide bonds and by hydrophobic interactions of the C_{H}3 domains. The Fc region of the antibody is responsible for various effector functions such as antibody-dependent cell-mediated cytotoxicity (ADCC), and complement dependent cytotoxicity (CDC), but does not function in antigen binding.

“Single-chain Fv-Fc antibody” or “scFv-Fc” refers to an engineered antibody consisting of a scFv connected to the Fc region of an antibody.

A “dsFv” refers to a disulfide-stabilized Fv fragment that the linkage between the variable domain of a single light chain and the variable domain of a single heavy chain is a disulfide bond. In some embodiments, a “(dsFv)₂” or “(dsFv-dsFv′)” comprises three peptide chains: two V_{H} domains linked by a peptide linker (e.g., a long flexible linker) and bound to two V_{L} domains, respectively, via disulfide bridges. In some embodiments, dsFv-dsFv′ is bispecific in which each disulfide paired heavy and light chain has a different antigen specificity.

“Camelized single domain antibody,” “heavy chain antibody,” or “HCAb” refers to an antibody that contains two V_{H} domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature (1993) 363:446-8; Nguyen V K. et al., Immunogenetics (2002) 54:39-47; Nguyen V K. et al., Immunology (2003) 109:93-101). The variable domain of a heavy chain antibody (VHH domain) represents the smallest known antigen-binding unit generated by adaptive immune responses (Koch-Nolte F. et al., FASEB J. (2007) 21:3490-8).

A “nanobody” refers to an antibody fragment that consists of a VHH domain from a heavy chain antibody and two constant domains, CH2 and CH3.

“Diabodies” or “dAbs” include small antibody fragments with two antigen-binding sites, wherein the fragments comprise a V_{H} domain connected to a V_{L} domain in the same polypeptide chain (V_{H}-V_{L} or V_{L}-V_{H}) (see, e.g., Holliger P. et al., Proc Natl Acad Sci USA. July 15; 90(14):6444-8 (1993); EP404097; WO93/11161). By using a linker that is too short to allow pairing between the two domains on the same chain, the domains are forced to pair with the complementary domains of another chain, thereby creating two antigen-binding sites. The antigen-binding sites may target the same or different antigens (or epitopes). In certain embodiments, a “bispecific ds diabody” is a diabody target two different antigens (or epitopes).

In certain embodiments, an “scFv dimer” is divalent (or bivalent) single-chain variable fragments (di-scFvs, bi-scFvs) that can be engineered by linking two scFvs. A bivalent diabody or bivalent scFv (BsFv, di-scFvs, bi-scFvs) comprising V_{H}-V_{L} (linked by a peptide linker) dimerized with another V_{H}-V_{L} moiety such that V_{H}'s of one moiety coordinate with the V_{L}'s of the other moiety and form two binding sites which can target the same antigens (or epitopes) or different antigens (or epitopes). In other embodiments, an “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate and each coordinated pair has a different antigen specificity.

A “domain antibody” refers to an antibody fragment containing only the variable domain of a heavy chain or the variable domain of a light chain. In certain instances, two or more V_{H} domains are covalently joined with a peptide linker to create a bivalent or multivalent domain antibody. The two V_{H} domains of a bivalent domain antibody may target the same or different antigens.

A “bispecific” antibody refers to an artificial antibody which has fragments derived from two different monoclonal antibodies and is capable of binding to two different epitopes. The two epitopes may present on the same antigen, or they may present on two different antigens.

“Cancer” as used herein refers to any medical condition characterized by malignant cell growth or neoplasm, abnormal proliferation, infiltration or metastasis, and includes both solid tumors and non-solid cancers (hematologic malignancies) such as leukemia. As used herein “solid tumor” refers to a solid mass of neoplastic and/or malignant cells. Examples of cancer or tumors include hematological malignancies, oral carcinomas (for example of the lip, tongue or pharynx), digestive organs (for example esophagus, stomach, small intestine, colon, large intestine, or rectum), peritoneum, liver and biliary passages, pancreas, respiratory system such as larynx or lung (small cell and non-small cell), bone, connective tissue, skin (e.g., melanoma), breast, reproductive organs (fallopian tube, uterus, cervix, testicles, ovary, or prostate), urinary tract (e.g., bladder or kidney), brain and endocrine glands such as the thyroid. In certain embodiments, the cancer is selected from ovarian cancer, breast cancer, head and neck cancer, renal cancer, bladder cancer, hepatocellular cancer, and colorectal cancer. In certain embodiments, the cancer is selected from a lymphoma, Hodgkin's lymphoma, non-Hodgkin's lymphoma and B-cell lymphoma.

The term “chimeric” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human animal, such as from mouse or rabbit. In some embodiments, the non-human animal is a mammal, for example, a mouse, a rat, a rabbit, a goat, a sheep, a guinea pig, or a hamster.

The term “specific binding” or “specifically binds” as used herein refers to a non-random binding reaction between two molecules, such as for example between an antibody and an antigen. In certain embodiments, the antibodies or antigen-binding fragments provided herein specifically bind to human LILRB4 with a binding affinity (K_{D}) of ≤10⁻⁶ M (e.g., ≤5×10⁻⁷ M, ≤2×10⁻⁷ M, ≤10⁻⁷ M, ≤5×10⁻⁸ M, ≤2×10⁻⁸ M, ≤10⁻⁸ M, ≤5×10⁻⁹ M, ≤4×10⁻⁹M, ≤3×10⁻⁹M, ≤2×10⁻⁹ M, or ≤10⁻⁹ M). K_{D} used herein refers to the ratio of the dissociation rate to the association rate (k_{off}/kₒₙ), which may be determined by using any conventional method known in the art, including but are not limited to surface plasmon resonance method, microscale thermophoresis method, HPLC-MS method and flow cytometry (such as FACS) method. In certain embodiments, the K_{D} value can be appropriately determined by using flow cytometry.

The ability to “block binding” or to “compete for the same epitope” as used herein refers to the ability of an antibody or antigen-binding fragment to inhibit the binding interaction between two molecules (e.g. human LILRB4 and an anti-LILRB4 antibody) to any detectable degree. In certain embodiments, an antibody or antigen-binding fragment that blocks binding between two molecules inhibits the binding interaction between the two molecules by at least 85%, or at least 90%. In certain embodiments, this inhibition may be greater than 85%, or greater than 90%.

Those skilled in the art will recognize that it is possible to determine, without undue experimentation, if a given antibody binds to the same epitope as the antibody of present disclosure by ascertaining whether the former prevents the latter from binding to a LILRB4 antigen polypeptide. If the given antibody competes with the antibody of present disclosure, as shown by a decrease in binding by the antibody of present disclosure to the LILRB4 antigen polypeptide, then the two antibodies bind to the same, or a closely related, epitope. Or if the binding of a given antibody to the LILRB4 antigen polypeptide was inhibited by the antibody of present disclosure, then the two antibodies bind to the same, or a closely related, epitope.

The term “chimeric antigen receptor” or “CAR”, as used herein, refer to engineered receptors that are capable of grafting a desired specificity to an antigen into immune effector cells, such as T cells, NK cells and macrophages. Typically, a CAR protein comprises an extracellular domain that introduces the desired specificity, a transmembrane domain and an intracellular domain that transmits a signal to the immune effector cells when the immune effector cells bind to the antigen. In certain embodiments, the extracellular domain comprises a leader peptide, an antigen recognition region and a spacer region. In certain embodiments, the antigen recognition region is derived from an antibody that specifically binds to the antigen. In certain embodiments, the antigen recognition region is a single-chain variable fragment (scFv) derived from the antibody. In certain embodiments, the single-chain variable fragment (scFv) is derived from a humanized antibody. In certain embodiment, the single-chain variable fragment comprises a heavy chain variable region fused to a light chain variable region through a flexible linker.

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g. Met, Ala, Val, Leu, and Ile), among residues with neutral hydrophilic side chains (e.g. Cys, Ser, Thr, Asn and Gln), among residues with acidic side chains (e.g. Asp, Glu), among amino acids with basic side chains (e.g. His, Lys, and Arg), or among residues with aromatic side chains (e.g. Trp, Tyr, and Phe). As known in the art, conservative substitution usually does not cause significant change in the protein conformational structure, and therefore could retain the biological activity of a protein.

“Effector functions” as used herein refer to biological activities attributable to the binding of Fc region of an antibody to its effectors such as C1 complex and Fc receptor. Exemplary effector functions include: complement dependent cytotoxicity (CDC) induced by interaction of antibodies and C1q on the C1 complex; antibody-dependent cell-mediated cytotoxicity (ADCC) induced by binding of Fc region of an antibody to Fc receptor on an effector cell; and phagocytosis.

The term “epitope” as used herein refers to the specific group of atoms or amino acids on an antigen to which an antibody binds. Two antibodies may bind the same or a closely related epitope within an antigen if they exhibit competitive binding for the antigen. For example, if an antibody or antigen-binding fragment blocks binding of a reference antibody to the antigen by at least 85%, or at least 90%, or at least 95%, then the antibody or antigen-binding fragment may be considered to bind the same/closely related epitope as the reference antibody.

The term “homologue” and “homologous” as used herein are interchangeable and refer to nucleic acid sequences (or its complementary strand) or amino acid sequences that have sequence identity of at least 80% (e.g., at least 85%, 88%, 90%, 91%, 92%, 93%, 94%, 95%, 96%, 97%, 98%, 99%) to another sequences when optimally aligned.

The phrase “host cell” as used herein refers to a cell into which an exogenous polynucleotide and/or a vector has been introduced.

The term “humanized” as used herein means that the antibody or antigen-binding fragment comprises CDRs derived from non-human animals, FR regions derived from human, and when applicable, the constant regions derived from human.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. An “isolated nucleic acid sequence” refers to the sequence of an isolated nucleic acid molecule. In certain embodiments, an “isolated antibody or antigen-binding fragment thereof” refers to the antibody or antigen-binding fragments having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% as determined by electrophoretic methods (such as SDS-PAGE, isoelectric focusing, capillary electrophoresis), or chromatographic methods (such as ion exchange chromatography or reverse phase HPLC).

A “leader peptide” refers to a peptide having a length of about 5-30 amino acids that is present at the N-terminus of newly synthesized proteins that form part of the secretory pathway. Proteins of the secretory pathway include, but are not limited to proteins that reside either inside certain organelles (the endoplasmic reticulum, Golgi or endosomes), are secreted from the cell, or are inserted into a cellular membrane. In some embodiments, the leader peptide forms part of the transmembrane domain of a protein.

“LILRB4” as used herein, refers to LILRB4 derived from any vertebrate source, including mammals such as primates (e.g., humans, monkeys) and rodents (e.g., mice and rats). Exemplary sequence of human LILRB4 includes GenBank SEQ Reference No. NP_001265355, AAH26309, ABM83015, ABM86208, AIC55892. The term “LILRB4” as used herein is intended to encompass any form of human LILRB4, for example, 1) native unprocessed LILRB4 molecule, “full-length” LILRB4 chain or naturally occurring variants of LILRB4, including, for example, splice variants or allelic variants; 2) any form of LILRB4 that results from processing in the cell; or 3) full length, a fragment (e.g., a truncated form, an extracellular/transmembrane domain) or a modified form (e.g. a mutated form, a glycosylated/PEGylated, a His-tag/immunofluorescence fused form) of LILRB4 subunit generated through recombinant method.

The term “anti-LILRB4 antibody” refers to an antibody that is capable of specifically binding to LILRB4 (e.g. human or monkey LILRB4).

A “LILRB4-related” disease or condition as used herein refers to any disease or condition caused by, exacerbated by, or otherwise linked to increased or decreased expression or activities of LILRB4. In some embodiments, the LILRB4 related condition is immune-related disorder, such as, for example, cancer, autoimmune disease, inflammatory disease or infectious disease.

The term “link” as used herein refers to the association via intramolecular interaction, e.g., covalent bonds, metallic bonds, and/or ionic bonding, or inter-molecular interaction, e.g., hydrogen bond or noncovalent bonds.

The term “operably linked” refers to an arrangement of elements wherein the components so described are configured so as to perform their usual function. Thus, a given signal peptide that is operably linked to a polypeptide directs the secretion of the polypeptide from a cell. In the case of a promoter, a promoter that is operably linked to a coding sequence will direct the expression of the coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol. (1990) 215:403-410; Stephen F. et al., Nucleic Acids Res. (1997) 25:3389-3402), ClustalW2 (available on the website of European Bioinformatics Institute, see also, Higgins D. G. et al., Methods in Enzymology (1996) 266:383-402; Larkin M. A. et al., Bioinformatics (2007) 23:2947-8), and ALIGN or Megalign (DNASTAR) software. Those skilled in the art may use the default parameters provided by the tool, or may customize the parameters as appropriate for the alignment, such as for example, by selecting a suitable algorithm.

The term “polynucleotide” or “nucleic acid” includes both single-stranded and double-stranded nucleotide polymers. The nucleotides comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate.

The term “polypeptide” or “protein” means a string of at least two amino acids linked to one another by peptide bonds. Polypeptides and proteins may include moieties in addition to amino acids (e.g., may be glycosylated) and/or may be otherwise processed or modified. Those of ordinary skill in the art will appreciate that a “polypeptide” or “protein” can be a complete polypeptide chain as produced by a cell (with or without a signal sequence), or can be a functional portion thereof. Those of ordinary skill will further appreciate that a polypeptide or protein can sometimes include more than one polypeptide chain, for example linked by one or more disulfide bonds or associated by other means. The term also includes amino acid polymers in which one or more amino acids are chemical analogs of a corresponding naturally-occurring amino acid and polymers.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

The term “therapeutically effective amount” or “effective dosage” as used herein refers to the dosage or concentration of a drug effective to treat a disease or condition. For example, with regard to the use of the monoclonal antibodies or antigen-binding fragments thereof disclosed herein to treat cancer, a therapeutically effective amount is the dosage or concentration of the monoclonal antibody or antigen-binding fragment thereof capable of reducing the tumor volume, eradicating all or part of a tumor, inhibiting or slowing tumor growth or cancer cell infiltration into other organs, inhibiting growth or proliferation of cells mediating a cancerous condition, inhibiting or slowing tumor cell metastasis, ameliorating any symptom or marker associated with a tumor or cancerous condition, preventing or delaying the development of a tumor or cancerous condition, or some combination thereof.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g., expression vectors) containing the nucleic acid sequence provided herein encoding the antibody or antigen-binding fragment thereof, at least one promoter (e.g., SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

### II. Anti-LILRB4 Antibody and Antigen-Binding Fragment

The present disclosure in one aspect provides an anti-LILRB4 antibody and antigen-binding fragment thereof that has a high binding affinity to LILRB4. In some embodiments, when bound to LILRB4, such antibodies modulate the activation of LILRB4. In certain embodiments, the antibody or antigen-binding fragment, when bound to LILRB4, suppresses activation of LILRB4. In certain embodiments, the antibody or antigen-binding fragment, when bound to LILRB4, can specifically interfere with, block or reduce the interaction between ApoE and LILRB4. In certain embodiments, the antibody or antigen-binding fragment provided herein is capable of inhibiting ApoE-mediated activity of LILRB4. In certain embodiments, the antibodies or antigen-binding fragments provided herein specifically or selectively bind to human LILRB4.

Binding affinity of the antibody and antigen-binding fragment provided herein can be represented by K_{D} value, which represents the ratio of dissociation rate to association rate (k_{off}/kₒₙ) when the binding between the antigen and antigen-binding molecule reaches equilibrium. The antigen-binding affinity (e.g., K_{D}) can be appropriately determined using suitable methods known in the art, including, for example, bio-layer interferometry.

Binding of the antibodies to human LILRB4 can also be represented by “half maximal effective concentration” (EC₅₀) value, which refers to the concentration of an antibody where 50% of its maximal effect (e.g., binding or inhibition etc.) is observed. The EC₅₀ value can be measured by methods known in the art, for example, sandwich assay such as ELISA, Western Blot, flow cytometry assay, and other binding assays.

### Specific Anti-LILRB4 Antibodies

The present disclosure in one aspect provides an anti-LILRB4 antibody and antigen-binding fragment thereof comprising one or more (e.g. 1, 2, 3, 4, 5, or 6) CDR sequences of an anti-LILRB4 antibody disclosed herein. CDRs are known to be responsible for antigen binding, however, it has been found that not all of the 6 CDRs are indispensable or unchangeable. In other words, it is possible to replace or change or modify one or more CDRs in anti-LILRB4 antibody disclosed herein, yet substantially retain the specific binding affinity to LILRB4.

In certain embodiments, the LILRB4 antibody is derived from the antibody H7K3 having heavy chain variable region sequence of SEQ ID NO: 1 and light chain variable region sequence of SEQ ID NO: 3. In particular embodiments, the anti-LILRB4 antibody has enhanced stability compared to H7K3, yet substantially retain the specific binding affinity to LILRB4.

In certain embodiments, the LILRB4 antibody has a heavy chain variable region comprising a heavy chain complementarity determining region (HC-CDR) 1 having an amino acid sequence of SEQ ID NO: 5, an HC CDR2 having an amino acid sequence of SEQ ID NO: 6 and an HC CDR3 having an amino acid sequence of SEQ ID NO: 7. In certain embodiments, the HC-CDR3 has an amino acid sequence of SEQ ID NO: 7 with a mutation at amino acid residue W.

In certain embodiments, the LILRB4 antibody has a light chain variable region comprising a light chain complementarity determining region (LC-CDR) 1 having an amino acid sequence of SEQ ID NO: 8, an LC-CDR2 having an amino acid sequence of SEQ ID NO: 9 and an LC-CDR3 having an amino acid sequence of SEQ ID NO: 10. In certain embodiments, the light chain variable region comprises an LC-CDR1 has an amino acid sequence of SEQ ID NO: 8 with a mutation at amino acid residues NS, an LC-CDR2 having an amino acid sequence of SEQ ID NO: 9 and an LC-CDR3 having an amino acid sequence of SEQ ID NO: 10. In certain embodiments, the LC-CDR1 has an amino acid sequence selected from SEQ ID NOs: 20, 22, 24, 26, 28, 30, 32, 34,

In certain embodiments, the LILRB4 antibody has a CDR sequence as listed in Table 1 below.

TABLE 1
CDR sequences of the anti-LILRB4 antibodies

| Antibody | | | |
|---|---|---|---|
| VH/VL # | CDR1 | CDR2 | CDR3 |
| H7 | SEQ ID NO: 5 | SEQ ID NO: 6 | SEQ ID NO: 7 |
| | GFSLSSSYW | IDSGSVGIT | ARHGDNWALDL |
| K3 | SEQ ID NO: 8 | SEQ ID NO: 9 | SEQ ID NO: 10 |
| | QSINSW | KASTLAS | HGYIRGDLDNV |
| H7m1 | SEQ ID NO: 5 | SEQ ID NO: 6 | SEQ ID NO: 12 |
| | GFSLSSSYW | IDSGSVGIT | ARHGDNVALDL |
| H7m2 | SEQ ID NO: 5 | SEQ ID NO: 6 | SEQ ID NO: 14 |
| | GFSLSSSYW | IDSGSVGIT | ARHGDNYALDL |
| H7m3 | SEQ ID NO: 5 | SEQ ID NO: 6 | SEQ ID NO: 16 |
| | GFSLSSSYW | IDSGSVGIT | ARHGDNFALDL |
| H7m4 | SEQ ID NO: 5 | SEQ ID NO: 6 | SEQ ID NO: 18 |
| | GFSLSSSYW | IDSGSVGIT | ARHGDNQALDL |
| K3m1 | SEQ ID NO: 20 | SEQ ID NO: 9 | SEQ ID NO: 10 |
| | QSIVSW | KASTLAS | HGYIRGDLDNV |
| K3m2 | SEQ ID NO: 22 | SEQ ID NO: 9 | SEQ ID NO: 10 |
| | QSIDSW | KASTLAS | HGYIRGDLDNV |
| K3m3 | SEQ ID NO: 24 | SEQ ID NO: 9 | SEQ ID NO: 10 |
| | QSIESW | KASTLAS | HGYIRGDLDNV |
| K3m4 | SEQ ID NO: 26 | SEQ ID NO: 9 | SEQ ID NO: 10 |
| | QSIQSW | LLIYKASTLAS | HGYIRGDLDNV |
| K3m5 | SEQ ID NO: 28 | SEQ ID NO: 9 | SEQ ID NO: 10 |
| | QSISSW | KASTLAS | HGYIRGDLDNV |
| K3m6 | SEQ ID NO: 30 | SEQ ID NO: 9 | SEQ ID NO: 10 |
| | QSITSW | KASTLAS | HGYIRGDLDNV |
| K3m7 | SEQ ID NO: 32 | SEQ ID NO: 9 | SEQ ID NO: 10 |
| | QSINQW | KASTLAS | HGYIRGDLDNV |
| K3m8 | SEQ ID NO: 34 | SEQ ID NO: 9 | SEQ ID NO: 10 |
| | QSINVW | KASTLAS | HGYIRGDLDNV |

The heavy chain and light chain variable region amino acid sequences of the anti-LILRB4 antibodies above are provided below.

H7
(SEQ ID NO: 1)
EVQLVESGGGLVQPGGSLRLSCAASGFSLSSSYWISWVRQAPGKGLE
WIGSIDSGSVGITYYATWVKGRFTISRDNSKNTLYLQMNSLRAEDTAVYY
CARHGDNWALDLWGQGTLVTVSS
K3
(SEQ ID NO: 3)
DIQMTQSPSTLSASVGDRVTITCRASQSINSWLAWYQQKPGKAPKLLIYK
ASTLASGVPSRFSGSGSGTEFTLTISSLQPDDFATYYCQHGYIRGDLDNV
FGGGTKVEIK
H7m1
(SEQ ID NO: 11)
EVQLVESGGGLVQPGGSLRLSCAASGFSLSSSYWISWVRQAPGKGLE
WIGSIDSGSVGITYYATWVKGRFTISRDNSKNTLYLQMNSLRAEDTAVYY
CARHGDNVALDLWGQGTLVTVSS
H7m2
(SEQ ID NO: 13)
EVQLVESGGGLVQPGGSLRLSCAASGFSLSSSYWISWVRQAPGKGLEWIG
SIDSGSVGITYYATWVKGRFTISRDNSKNTLYLQMNSLRAEDTAVYYC
ARHGDNYALDLWGQGTLVTVSS
H7m3
(SEQ ID NO: 15)
EVQLVESGGGLVQPGGSLRLSCAASGFSLSSSYWISWVRQAPGKGLE
WIGSIDSGSVGITYYATWVKGRFTISRDNSKNTLYLQMNSLRAEDTAVYY
CARHGDNFALDLWGQGTLVTVSS
H7m4
(SEQ ID NO: 17)
EVQLVESGGGLVQPGGSLRLSCAASGFSLSSSYWISWVRQAPGKGLE
WIGSIDSGSVGITYYATWVKGRFTISRDNSKNTLYLQMNSLRAEDTAVYY
CARHGDNQALDLWGQGTLVTVSS
K3m1
(SEQ ID NO: 19)
DIQMTQSPSTLSASVGDRVTITCRASQSIVSWLAWYQQKPGKAPKLLIYK
ASTLASGVPSRFSGSGSGTEFTLTISSLQPDDFATYYCQHGYIRGDLDNV
FGGGTKVEIK
K3m2
(SEQ ID NO: 21)
DIQMTQSPSTLSASVGDRVTITCRASQSIDSWLAWYQQKPGKAPKLLIYK
ASTLASGVPSRFSGSGSGTEFTLTISSLQPDDFATYYCQHGYIRGDLDNV
FGGGTKVEIK
K3m3
(SEQ ID NO: 23)
DIQMTQSPSTLSASVGDRVTITCRASQSIESWLAWYQQKPGKAPKLLIYK
ASTLASGVPSRFSGSGSGTEFTLTISSLQPDDFATYYCQHGYIRGDLDNV
FGGGTKVEIK
K3m4
(SEQ ID NO: 25)
DIQMTQSPSTLSASVGDRVTITCRASQSIQSWLAWYQQKPGKAPKLLIYK
ASTLASGVPSRFSGSGSGTEFTLTISSLQPDDFATYYCQHGYIRGDLDNV
FGGGTKVEIK
K3m5
(SEQ ID NO: 27)
DIQMTQSPSTLSASVGDRVTITCRASQSISSWLAWYQQKPGKAPKLLIYK
ASTLASGVPSRFSGSGSGTEFTLTISSLQPDDFATYYCQHGYIRGDLDNV
FGGGTKVEIK
K3m6
(SEQ ID NO: 29)
DIQMTQSPSTLSASVGDRVTITCRASQSITSWLAWYQQKPGKAPKLLIYK
ASTLASGVPSRFSGSGSGTEFTLTISSLQPDDFATYYCQHGYIRGDLDNV
FGGGTKVEIK
K4m7
(SEQ ID NO: 31)
DIQMTQSPSTLSASVGDRVTITCRASQSINQWLAWYQQKPGKAPKLLIYK
ASTLASGVPSRFSGSGSGTEFTLTISSLQPDDFATYYCQHGYIRGDLDNV
FGGGTKVEIK
K4m8
(SEQ ID NO: 33)
DIQMTQSPSTLSASVGDRVTITCRASQSINVWLAWYQQKPGKAPKLLIYK
ASTLASGVPSRFSGSGSGTEFTLTISSLQPDDFATYYCQHGYIRGDLDNV
FGGGTKVEIK

In certain embodiments, the antibodies and antigen-binding fragments thereof provided herein comprise suitable framework region (FR) sequences, as long as the antibodies and antigen-binding fragments thereof can specifically bind to LILRB4. The CDR sequences provided in Table 1 can be grafted to any suitable FR sequences of any suitable species such as mouse, human, rat, rabbit, among others, using suitable methods known in the art such as recombinant techniques.

In certain embodiments, the antibodies and antigen-binding fragments thereof provided herein are humanized. A humanized antibody or antigen-binding fragment is desirable in its reduced immunogenicity in human. A humanized antibody is chimeric in its variable regions, as non-human CDR sequences are grafted to human or substantially human FR sequences. Humanization of an antibody or antigen-binding fragment can be essentially performed by substituting the non-human (such as murine) CDR genes for the corresponding human CDR genes in a human immunoglobulin gene (see, for example, Jones et al., Nature (1986) 321:522-525; Riechmann et al., Nature (1988) 332:323-327; Verhoeyen et al., Science (1988) 239:1534-1536).

Suitable human heavy chain and light chain variable domains can be selected to achieve this purpose using methods known in the art. In an illustrative example, “best-fit” approach can be used, where a non-human (e.g. rodent) antibody variable domain sequence is screened or BLASTed against a database of known human variable domain sequences, and the human sequence closest to the non-human query sequence is identified and used as the human scaffold for grafting the non-human CDR sequences (see, for example, Sims et al., J. Immunol. (1993) 151:2296; Chothia et al., J. Mot. Biol. (1987) 196:901). Alternatively, a framework derived from the consensus sequence of all human antibodies may be used for the grafting of the non-human CDRs (see, for example, Carter et at. Proc. Natl. Acad. Sci. USA (1992) 89:4285; Presta et al., J. Immunol. (1993) 151:2623).

In certain embodiments, the humanized antibodies or antigen-binding fragments provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived different human immunoglobulin genes, for example, FR sequences derived from one human antibody and constant region from another human antibody.

In certain embodiments, the humanized antibodies and antigen-binding fragment thereof provided herein comprise a heavy chain FR sequence of H7 and/or a light chain FR sequence of K3. In some embodiments, the FR regions derived from human may comprise the same amino acid sequence as the human immunoglobulin from which it is derived. In some embodiments, one or more amino acid residues of the human FR are substituted with the corresponding residues from the parent non-human antibody. This may be desirable in certain embodiments to make the humanized antibody or its fragment closely approximate the non-human parent antibody structure. In certain embodiments, the humanized antibody or antigen-binding fragment provided herein comprises no more than 10, 9, 8, 7, 6, 5, 4, 3, 2, or 1 amino acid residue substitutions in each of the human FR sequences, or no more than 10, 9, 8, 7, 6, 5, 4, 3, 2, or 1 amino acid residue substitutions in all the FRs of a heavy or a light chain variable domain. In some embodiments, such change in amino acid residue could be present in heavy chain FR regions only, in light chain FR regions only, or in both chains.

In certain embodiments, the antibodies and antigen-binding fragments thereof provided herein comprise a heavy chain variable domain sequence selected from the group consisting of SEQ ID NOs: 1, 11, 13, 15, and 17. In certain embodiments, the antibodies and antigen-binding fragments thereof provided herein comprise a light chain variable domain sequence selected from the group consisting of SEQ ID NOs: 3, 19, 21, 23, 25, 27, 29, 31, and 33.

In some embodiments, the anti-LILRB4 antibodies and the antigen-binding fragments provided herein comprise all or a portion of the heavy chain variable domain and/or all or a portion of the light chain variable domain. In one embodiment, the anti-LILRB4 antibodies and the antigen-binding fragments provided herein is a single domain antibody which consists of all or a portion of the heavy chain variable domain provided herein. More information of such a single domain antibody is available in the art (see, e.g., U.S. Pat. No. 6,248,516).

In certain embodiments, the anti-LILRB4 antibodies and the fragments thereof provided herein further comprise an immunoglobulin constant region. In some embodiments, an immunoglobulin constant region comprises a heavy chain and/or a light chain constant region. The heavy chain constant region comprises CH1, hinge, and/or CH2-CH3 regions. In certain embodiments, the heavy chain constant region comprises an Fc region. In certain embodiments, the light chain constant region comprises Cκ or Cλ.

The antibodies or antigen-binding fragments thereof provided herein can be a monoclonal antibody, polyclonal antibody, humanized antibody, chimeric antibody, recombinant antibody, bispecific antibody, labeled antibody, bivalent antibody, or anti-idiotypic antibody. A recombinant antibody is an antibody prepared in vitro using recombinant methods rather than in animals.

### Antibody Variants

The antibodies and antigen-binding fragments thereof provided herein also encompass various variants thereof. In certain embodiments, the antibodies and antigen-binding fragments thereof encompasses various types of variants of an exemplary antibody provided herein.

In certain embodiments, the antibody variants comprise one or more modifications or substitutions in one or more CDR sequences as provided in Table 1, one or more variable region sequences (but not in any of the CDR sequences) provided herein, and/or the constant region (e.g., Fc region). Such variants retain specific binding affinity to LILRB4 of their parent antibodies, but have one or more desirable properties conferred by the modification(s) or substitution(s). For example, the antibody variants may have improved antigen-binding affinity, improved glycosylation pattern, reduced risk of glycosylation, reduced deamidation or deamination, improved or increased effector function(s), reduced or depleted effector function(s), improved FcRn receptor binding, increased pharmacokinetic half-life, pH sensitivity, and/or compatibility to conjugation (e.g., one or more introduced cysteine residues).

The parent antibody sequence may be screened to identify suitable or preferred residues to be modified or substituted, using methods known in the art, for example “alanine scanning mutagenesis” (see, for example, Cunningham and Wells (1989) Science, 244:1081-1085). Briefly, target residues (e.g., charged residues such as Arg, Asp, His, Lys, and Glu) can be identified and replaced by a neutral or negatively charged amino acid (e.g., alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g. cysteine residue, positively charged residue, etc.).

### Affinity Variant

Affinity variant may contain modifications or substitutions in one or more CDR sequences, one or more FR sequences, or the heavy or light chain variable region sequences provided herein. The affinity variants retain specific binding affinity to LILRB4 of the parent antibody, or even have improved LILRB4 specific binding affinity over the parent antibody.

Various methods known in the art can be used to achieve this purpose. For example, a library of antibody variants (such as Fab or scFv variants) can be generated and expressed with phage display technology, and then screened for the binding affinity to human LILRB4. For another example, computer software can be used to virtually simulate the binding of the antibodies to human LILRB4, and identify the amino acid residues on the antibodies which form the binding interface. Such residues may be either avoided in the substitution so as to prevent reduction in binding affinity or targeted for substitution to provide for a stronger binding.

In certain embodiments, the humanized antibody or antigen-binding fragment provided herein comprises one or more amino acid residue substitutions in one or more CDR sequences, and/or one or more FR sequences. In certain embodiments, an affinity variant comprises no more than 10, 9, 8, 7, 6, 5, 4, 3, 2, or 1 substitution in the CDR sequences and/or FR sequences in total.

In certain embodiments, the anti-LILRB4 antibodies and antigen-binding fragments thereof comprise 1, 2, or 3 CDR sequences having at least 80% (e.g. at least 85%, 88%, 90%, 91%, 92%, 93%, 94%, 95%, 96%, 97%, 98%, 99%) sequence identity to that (or those) listed in Table 1, and in the meantime retain the binding affinity to LILRB4 at a level similar to or even higher than its parent antibody.

In certain embodiments, the anti-LILRB4 antibodies and antigen-binding fragments thereof comprise one or more variable region sequences having at least 80% (e.g. at least 85%, 88%, 90%, 91%, 92%, 93%, 94%, 95%, 96%, 97%, 98%, 99%) sequence identity to that (or those) provided herein, and in the meantime retain the binding affinity to LILRB4 at a level similar to or even higher than its parent antibody. In some embodiments, the substitutions, insertions, or deletions occur in regions outside the CDRs (e.g., in the FRs).

### Glycosylation Variant

In still another embodiment, the antibody comprises a particular glycosylation pattern. For example, an aglycosylated antibody can be made (i.e., the antibody lacks glycosylation). The glycosylation pattern of an antibody may be altered to, for example, increase the affinity or avidity of the antibody for an antigen. Such modifications can be accomplished by, for example, altering one or more of the glycosylation sites within the antibody sequence. For example, one or more amino acid substitutions can be made that result removal of one or more of the variable region framework glycosylation sites to thereby eliminate glycosylation at that site. Such aglycosylation may increase the affinity or avidity of the antibody for antigen. See, e.g., U.S. Pat. Nos. 5,714,350 and 6,350,861.

An antibody may also be made in which the glycosylation pattern includes hypofucosylated or afucosylated glycans, such as a hypofucosylated antibodies or afucosylated antibodies have reduced amounts of fucosyl residues on the glycan. The antibodies may also include glycans having an increased amount of bisecting GlcNac structures. Such altered glycosylation patterns have been demonstrated to increase the ADCC ability of antibodies. Such modifications can be accomplished by, for example, expressing the antibodies in a host cell in which the glycosylation pathway was been genetically engineered to produce glycoproteins with particular glycosylation patterns. These cells have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (α(1,6)-fucosyltransferase), such that antibodies expressed in the Ms704, Ms705, and Ms709 cell lines lack fucose on their carbohydrates. The Ms704, Ms705, and Ms709 FUT8−/− cell lines were created by the targeted disruption of the FUT8 gene in CHO/DG44 cells using two replacement vectors (see U.S. Patent Publication No. 20040110704. As another example, EP 1 176 195 describes a cell line with a functionally disrupted FUT8 gene, which encodes a fucosyl transferase, such that antibodies expressed in such a cell line exhibit hypofucosylation by reducing or eliminating the α-1,6 bond-related enzyme. EP 1 176 195 also describes cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 2003/035835 describes a variant CHO cell line, Lec13 cells, with reduced ability to attach fucose to Asn(297)-linked carbohydrates, also resulting in hypofucosylation of antibodies expressed in that host cell. Antibodies with a modified glycosylation profile can also be produced in chicken eggs, as described in PCT Publication WO 06/089231. Alternatively, antibodies with a modified glycosylation profile can be produced in plant cells, such as Lemna (U.S. Pat. No. 7,632,983). Methods for production of antibodies in a plant system are disclosed in the U.S. Pat. Nos. 6,998,267 and 7,388,081. PCT Publication WO1999/054342 describes cell lines engineered to express glycoprotein-modifying glycosyl transferases (e.g., β(1,4)-N-acetylglucosaminyltransferase III (GnTIII)) such that antibodies expressed in the engineered cell lines exhibit increased bisecting GlcNac structures which results in increased ADCC activity of the antibodies. Hypofucosylation is also called afucosylation when fucosylation is minimal on antibodies.

Alternatively, the fucose residues of the antibodies can be cleaved off using a fucosidase enzyme; e.g., the fucosidase α-L-fucosidase removes fucosyl residues from antibodies. Antibodies disclosed herein further include those produced in lower eukaryote host cells, in particular fungal host cells such as yeast and filamentous fungi have been genetically engineered to produce glycoproteins that have mammalian- or human-like glycosylation patterns. A particular advantage of these genetically modified host cells over currently used mammalian cell lines is the ability to control the glycosylation profile of glycoproteins that are produced in the cells such that compositions of glycoproteins can be produced wherein a particular N-glycan structure predominates (see, e.g., U.S. Pat. Nos. 7,029,872 and 7,449,308). These genetically modified host cells have been used to produce antibodies that have predominantly particular N-glycan structures.

In addition, since fungi such as yeast or filamentous fungi lack the ability to produce fucosylated glycoproteins, antibodies produced in such cells will lack fucose unless the cells are further modified to include the enzymatic pathway for producing fucosylated glycoproteins (See for example, PCT Publication WO2008112092). In particular embodiments, the antibodies disclosed herein further include those produced in lower eukaryotic host cells and which comprise fucosylated and nonfucosylated hybrid and complex N-glycans, including bisected and multiantennary species, including but not limited to N-glycans such as GlcNAc(1-4)Man3GlcNAc2; Gal(1-4)GlcNAc(1-4)Man3GlcNAc2; NANA(1-4)Gal(1-4)GlcNAc(1-4)Man3GlcNAc2. In particular embodiments, the antibody compositions provided herein may comprise antibodies having at least one hybrid N-glycan selected from the group consisting of GlcNAcMan5GlcNAc2; GalGlcNAcMan5GlcNAc2; and NANAGalGlcNAcMan5GlcNAc2. In particular aspects, the hybrid N-glycan is the predominant N-glycan species in the composition. In further aspects, the hybrid N-glycan is a particular N-glycan species that comprises about 30%, 40%, 50%, 60%, 70%, 80%, 90%, 95%, 97%, 98%, 99%, or 100% of the hybrid N-glycans in the composition.

In particular embodiments, the antibody compositions provided herein comprise antibodies having at least one complex N-glycan selected from the group consisting of GlcNAcMan3GlcNAc2; GalGlcNAcMan3GlcNAc2; NANAGalGlcNAcMan3GlcNAc2; GlcNAc2Man3GlcNAc2; GalGlcNAc2Man3GlcNAc2; Gal2GlcNAc2Man3GlcNAc2; NANAGal2GlcNAc2Man3GlcNAc2; and NANA2Gal2GlcNAc2Man3GlcNAc2. In particular aspects, the complex N-glycan is the predominant N-glycan species in the composition. In further aspects, the complex N-glycan is a particular N-glycan species that comprises about 30%, 40%, 50%, 60%, 70%, 80%, 90%, 95%, 97%, 98%, 99%, or 100% of the complex N-glycans in the composition. In particular embodiments, the N-glycan is fusosylated. In general, the fucose is in an α1,3-linkage with the GlcNAc at the reducing end of the N-glycan, an α1,6-linkage with the GlcNAc at the reducing end of the N-glycan, an α1,2-linkage with the Gal at the non-reducing end of the N-glycan, an α1,3-linkage with the GlcNac at the non-reducing end of the N-glycan, or an α1,4-linkage with a GlcNAc at the non-reducing end of the N-glycan.

Therefore, in particular aspects of the above the glycoprotein compositions, the glycoform is in an α1,3-linkage or α1,6-linkage fucose to produce a glycoform selected from the group consisting of Man5GlcNAc2(Fuc), GlcNAcMan5GlcNAc2(Fuc), Man3GlcNAc2(Fuc), GlcNAcMan3GlcNAc2(Fuc), GlcNAc2Man3GlcNAc2(Fuc), GalGlcNAc2Man3GlcNAc2(Fuc), Gal2GlcNAc2Man3GlcNAc2(Fuc), NANAGal2GlcNAc2Man3GlcNAc2(Fuc), and NANA2Gal2GlcNAc2Man3GlcNAc2(Fuc); in an α1,3-linkage or α1,4-linkage fucose to produce a glycoform selected from the group consisting of GlcNAc(Fuc)Man5GlcNAc2, GlcNAc(Fuc)Man3GlcNAc2, GlcNAc2(Fuc1-2)Man3GlcNAc2, GalGlcNAc2(Fuc1-2)Man3GlcNAc2, Gal2GlcNAc2(Fuc1-2)Man3GlcNAc2, NANAGal2GlcNAc2(Fuc1-2)Man3GlcNAc2, and NANA2Gal2GlcNAc2(Fuc1-2)Man3GlcNAc2; or in an α1,2-linkage fucose to produce a glycoform selected from the group consisting of Gal(Fuc)GlcNAc2Man3GlcNAc2, Gal2(Fuc1-2)GlcNAc2Man3GlcNAc2, NANAGal2(Fuc1-2)GlcNAc2Man3GlcNAc2, and NANA2Gal2(Fuc1-2)GlcNAc2Man3GlcNAc2.

In further aspects, the antibodies comprise high mannose N-glycans, including but not limited to, Man8GlcNAc2, Man7GlcNAc2, Man6GlcNAc2, Man5GlcNAc2, Man4GlcNAc2, or N-glycans that consist of the Man3GlcNAc2 N-glycan structure. In further aspects of the above, the complex N-glycans further include fucosylated and non-fucosylated (or afucosylated) bisected and multiantennary species. As used herein, the terms “N-glycan” and “glycoform” are used interchangeably and refer to an N-linked oligosaccharide, for example, one that is attached by an asparagine-N-acetylglucosamine linkage to an asparagine residue of a polypeptide. N-linked glycoproteins contain an N-acetylglucosamine residue linked to the amide nitrogen of an asparagine residue in the protein.

The anti-LILRB4 antibodies and antigen-binding fragments provided herein also encompass a glycosylation variant, which can be obtained to either increase or decrease the extent of glycosylation of the antibody or antigen binding fragment.

The antibody or antigen binding fragment thereof may comprise one or more amino acid residues with a side chain to which a carbohydrate moiety (e.g. an oligosaccharide structure) can be attached. Glycosylation of antibodies is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine. Removal of a native glycosylation site can be conveniently accomplished, for example, by altering the amino acid sequence such that one of the above-described tripeptide sequences (for N-linked glycosylation sites) or serine or threonine residues (for O-linked glycosylation sites) present in the sequence in the is substituted. A new glycosylation site can be created in a similar way by introducing such a tripeptide sequence or serine or threonine residue.

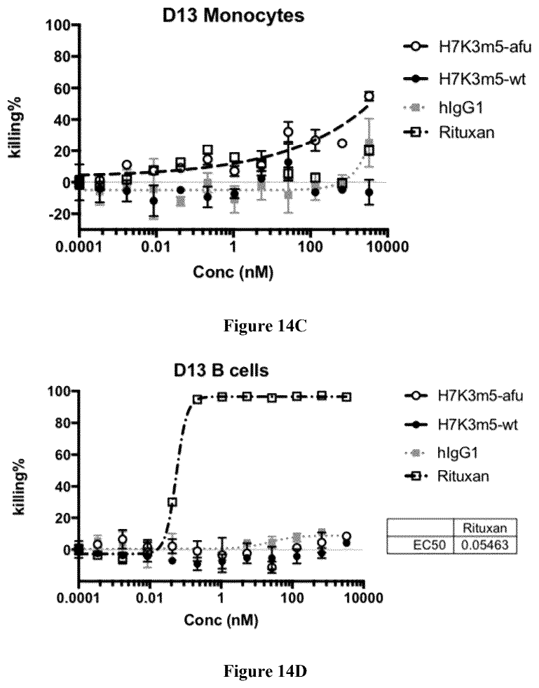

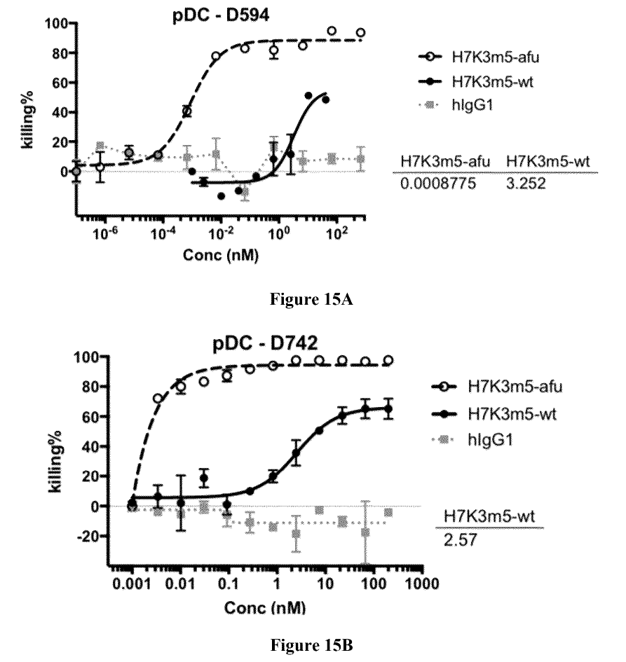

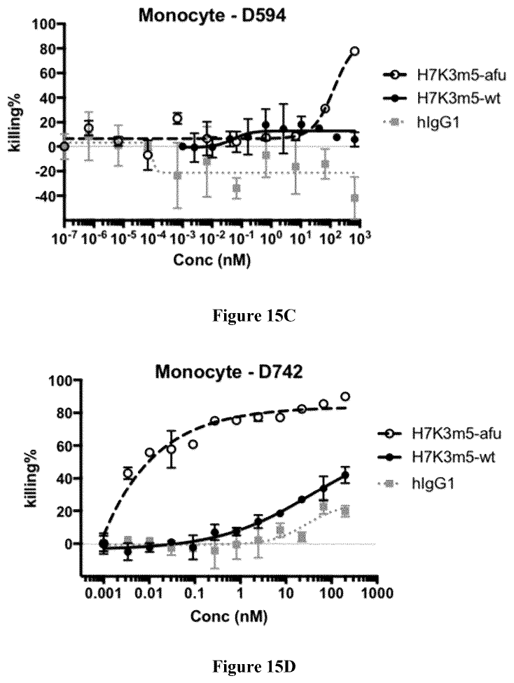

One type of glycosylation modification is made with antibody-producing cells deficient for certain enzymatic pathways that are responsible for site-specific glycosylation, including fucosylation. For example, an antibody lacking fucosylation (referred to as afucosylated antibody) generally has enhanced ADCC activity. With afucosylated H7K3m5, killing of normal monocytes via ADCC was observed in 25-50% of tested PBMC donors (FIGS. 14A-14D, and FIGS. 15C-15D). In addition, as shown in FIGS. 15A-15B, both afucosylated and wild type H7K3m5 resulted in killing of pDCs via autologous ADCC. In the meantime, monocytes may be killed only with afucosylated H7K3m5, depending on donors, and not with wild-type H7K3m5 (FIGS. 15C-15D).

### Cysteine-Engineered Variant

The anti-LILRB4 antibodies and antigen-binding fragments provided herein also encompass a cysteine-engineered variant, which comprises one or more introduced free cysteine amino acid residues.

A free cysteine residue is one which is not part of a disulfide bridge. A cysteine-engineered variant is useful for conjugation with for example, a cytotoxic and/or imaging compound, a label, or a radioisotope among others, at the site of the engineered cysteine, through for example a maleimide or haloacetyl. Methods for engineering antibodies or antigen-binding fragments to introduce free cysteine residues are known in the art, see, for example, WO2006/034488.

### Fc Variant

The antibodies disclosed herein can also be engineered to include modifications within the Fc region, typically to alter one or more functional properties of the antibody, such as serum half-life, complement fixation, Fc receptor binding, and/or effector function (e.g., antigen-dependent cellular cytotoxicity). Furthermore, the antibodies disclosed herein can be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat. The antibodies disclosed herein also include antibodies with modified (or blocked) Fc regions to provide altered effector functions. See, e.g., U.S. Pat. No. 5,624,821; WO2003/086310; US2004/0002587; US2005/0152894; US2005/0249723; WO2006/019447. Such modification can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies, enabling less frequent dosing and thus increased convenience and decreased use of material. This mutation has been reported to abolish the heterogeneity of inter-heavy chain disulfide bridges in the hinge region.

In one embodiment, the hinge region of CH1 is modified such that the number of cysteine residues in the hinge region is increased or decreased. This approach is described further in U.S. Pat. No. 5,677,425. The number of cysteine residues in the hinge region of CH1 is altered, for example, to facilitate assembly of the light and heavy chains or to increase or decrease the stability of the antibody. In another embodiment, the antibody is modified to increase its biological half-life. Various approaches are possible. For example, one or more of the following mutations can be introduced: T252L, T254S, T256F, as described in U.S. Pat. No. 6,277,375. Alternatively, to increase the biological half-life, the antibody can be altered within the CH1 or CL region to contain a salvage receptor binding epitope taken from two loops of a C_{H}2 domain of an Fc region of an IgG, as described in U.S. Pat. Nos. 5,869,046 and 6,121,022. In yet other embodiments, the Fc region is altered by replacing at least one amino acid residue with a different amino acid residue to alter the effector function(s) of the antibodies. For example, one or more amino acids selected from amino acid residues 234, 235, 236, 237, 297, 318, 320 and 322 can be replaced with a different amino acid residue such that the antibody has an altered affinity for an effector ligand but retains the antigen binding ability of the parent antibody. The effector ligand to which affinity is altered can be, for example, an Fc receptor or the C1 component of complement. This approach is described in further detail in U.S. Pat. Nos. 5,624,821 and 5,648,260.

In another example, one or more amino acid residues within amino acid positions 231 and 239 are altered to thereby alter the ability of the antibody to fix complement. This approach is described further in PCT Publication WO1994/029351. In yet another example, the Fc region is modified to increase or decrease the ability of the antibodies to mediate antibody dependent cellular cytotoxicity (ADCC) and/or to increase or decrease the affinity of the antibodies for an Fc receptor by modifying one or more amino acids at the following positions: 238, 239, 243, 248, 249, 252, 254, 255, 256, 258, 264, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 2000/042072. Moreover, the binding sites on human IgG1 for FcγR1, FcγRII, FcγRIII and FcRn have been mapped and variants with improved binding have been described. Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A.

In one embodiment, the Fc region is modified to decrease the ability of the antibodies to mediate effector function and/or to increase anti-inflammatory properties by modifying residues 243 and 264. In one embodiment, the Fc region of the antibody is modified by changing the residues at positions 243 and 264 to alanine. In one embodiment, the Fc region is modified to decrease the ability of the antibody to mediate effector function and/or to increase anti-inflammatory properties by modifying residues 243, 264, 267 and 328.

In one embodiment, the Fc region is modified to abolish the ability of the antibodies to mediate effector function by modifying residues 234, 235 and 329 to alanine or glycine (L234A-L235A-P329G).

The anti-LILRB4 antibodies and antigen-binding fragments provided herein also encompass an Fc variant, which comprises one or more amino acid residue modifications or substitutions at its Fc region and/or hinge region.

In certain embodiments, the anti-LILRB4 antibodies or antigen-binding fragments comprise one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody and antigen-binding fragment thereof to improve binding affinity with FcRn are well-known in the art, see, for example, Vaughn, D. et al., Structure, 6(1): 63-73, 1998; Kontermann, R. et al., Antibody Engineering, Volume 1, Chapter 27: Engineering of the Fc region for improved PK, published by Springer, 2010; Yeung, Y. et al., Cancer Research (2010) 70: 3269-3277; and Hinton, P. et al., J. Immunology (2006) 176:346-356.

In certain embodiments, the anti-LILRB4 antibodies or antigen-binding fragments comprise one or more amino acid substitution(s) that alters the antibody-dependent cellular cytotoxicity (ADCC). Certain amino acid residues at CH2 domain of the Fc region can be substituted to provide for enhanced ADCC activity. Alternatively, or additionally, carbohydrate structures on the antibody can be changed to enhance ADCC activity. Methods of altering ADCC activity by antibody engineering have been described in the art, see for example, Shields R L. et al., J Biol Chem. (2001) 276(9): 6591-604; Idusogie E E. et al., J Immunol. (2000) 164(8):4178-84; Steurer W. et al., J Immunol. (1995) 155(3): 1165-74; Idusogie E E. et al., J Immunol. (2001) 166(4): 2571-5; Lazar G A. et al., PNAS (2006) 103(11): 4005-4010; Ryan M C. et al., Mol. Cancer Ther. (2007) 6: 3009-3018; Richards J O. et al., Mol Cancer Ther. (2008) 7(8): 2517-27; Shields R. L. et al., J. Biol. Chem, 2002, 277: 26733-26740; Shinkawa T. et al., J. Biol. Chem (2003) 278: 3466-3473.

In certain embodiments, the anti-LILRB4 antibodies or antigen-binding fragments comprise one or more amino acid substitution(s) that alters Complement Dependent Cytotoxicity (CDC), for example, by improving or diminishing C1q binding and/or CDC (see, for example, WO99/51642; Duncan & Winter Nature 322:738-40 (1988); U.S. Pat. Nos. 5,648,260; 5,624,821); and WO1994/029351 concerning other examples of Fe region variants.

In certain embodiments, the anti-LILRB4 antibodies or antigen-binding fragments comprise one or more amino acid substitution(s) in the interface of the Fc region to facilitate and/or promote heterodimerization. These modifications comprise introduction of a protuberance into a first Fc polypeptide and a cavity into a second Fc polypeptide, wherein the protuberance can be positioned in the cavity so as to promote interaction of the first and second Fc polypeptides to form a heterodimer or a complex. Methods of generating antibodies with these modifications are known in the art, e.g., as described in U.S. Pat. No. 5,731,168.

### Antigen-Binding Fragments

Provided herein are also anti-LILRB4 antigen-binding fragments. Various types of antigen-binding fragments are known in the art and can be developed based on the anti-LILRB4 antibodies provided herein, including for example, the exemplary antibodies whose CDR and variable sequences are provided herein, and their different variants (such as affinity variants, glycosylation variants, Fc variants, cysteine-engineered variants and so on).

In certain embodiments, an anti-LILRB4 antigen-binding fragment provided herein is a camelized single domain antibody, a diabody, a single chain Fv fragment (scFv), an scFv dimer, a BsFv, a dsFv, a (dsFv)₂, a dsFv-dsFv′, an Fv fragment, a Fab, a Fab′, a F(ab′)₂, a bispecific antibody, a ds diabody, a nanobody, a domain antibody, a single domain antibody, or a bivalent domain antibody.

A Single Chain Variable Fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alaine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the VH C terminus and an abundance of arginines and prolines at other positions as the only common features of the selected tethers.

The recombinant antibodies of the present disclosure may also involve sequences or moieties that permit dimerization or multimerization of the receptors. Such sequences include those derived from IgA, which permit formation of multimers in conjunction with the J-chain. Another multimerization domain is the Gal4 dimerization domain. In other embodiments, the chains may be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., Nhydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338 describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

Various techniques can be used for the production of such antigen-binding fragments. Illustrative methods include, enzymatic digestion of intact antibodies (see, e.g., Morimoto et al., Journal of Biochemical and Biophysical Methods (1992) 24:107-117; and Brennan et al., Science (1985) 229:81), recombinant expression by host cells such as E. coli (e.g. for Fab, Fv and ScFv antibody fragments), screening from a phase display library as discussed above (e.g. for ScFv), and chemical coupling of two Fab′-SH fragments to form F(ab′)₂ fragments (Carter et al., Bio/Technology (1992) 10:163-167). Other techniques for the production of antibody fragments will be apparent to a skilled practitioner.

In certain embodiments, the antigen-binding fragment is a scFv. Generation of scFv is described in, for example, WO 93/16185; U.S. Pat. Nos. 5,571,894; and 5,587,458. scFv may be fused to an effector protein at either the amino or the carboxyl terminus to provide for a fusion protein (see, for example, Antibody Engineering, ed. Borrebaeck).

### Bispecific Antibodies

In certain embodiments, the LILRB4 antibody disclosed herein is a bispecific antibody. In certain embodiments, the LILRB4 bispecific antibody can be used to treat hematologic and solid malignancies via redirecting the cytotoxic T-cells or NK-cells toward cancer cells by engaging both antigens on the T-cells or NK-cells and LILRB4 on the cancer cells. In some embodiments, the anti-LILRB4 bispecific antibody is against a T-cell receptor such as CD3. In some embodiments, the anti-LILRB4 bispecific antibody is against an NK-cell receptor such as CD16A.

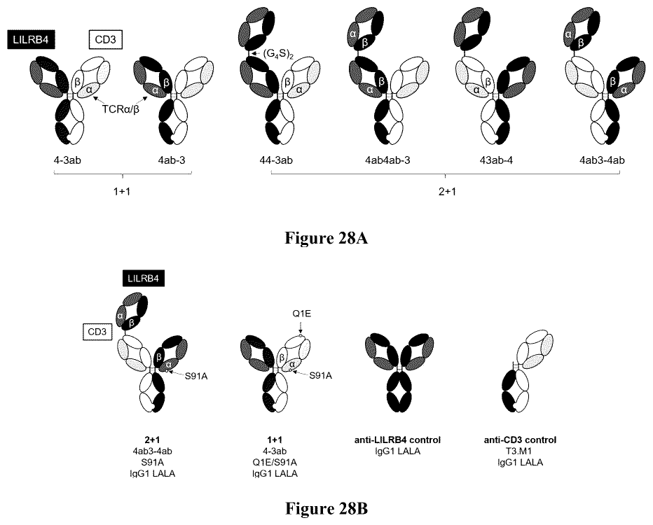

It is appreciated that the anti-LILRB4 bispecific antibodies of the present disclosure can have various forms and structures, which can be understood by the exemplary embodiments of the bispecific antibody specifically binds to LILRB4 and CD3 (LILRB4/CD3 bispecific) as illustrated in FIGS. 28A and 28B.

As illustrated in FIG. 28A, in an exemplary embodiment of the invention, the LILRB4/CD3 bispecific antibody is Y shaped and comprises two arms. One arm of the antibody, formed by a portion of a first heavy chain polypeptide and a first light chain polypeptide, comprises a pair of heavy chain variable domain (V_{H}1) and light chain variable domain (V_{L}l), which form an antigen-binding site capable of specifically binding to LILRB4. The other arm of the antibody, formed by a portion of a second heavy chain polypeptide and a second light chain polypeptide, comprises a second pair of heavy chain variable domains (V_{H}2) and light chain variable domain (V_{L}2). The V_{H}2 and V_{L}2 form a second antigen-binding site capable of specifically binding to CD3. In this configuration, each bispecific antibody comprises a single copy of the antigen binding site against LILRB4 and a single copy of the antigen binding site against CD3, which is referred to as 1+1

Referring to the embodiment 4-3ab in FIG. 28A, the first heavy chain polypeptide comprises from N to C terminus V_{H}1-C_{H}1-C_{H}2-C_{H}3, wherein C_{H}1, C_{H}2, and C_{H}3 refers to heavy chain constant domain 1, 2 and 3; the first light chain polypeptide comprises from N to C terminus V_{L}1-C_{L}, wherein C_{L} refers to a light chain constant domain; the second heavy chain polypeptide comprises from N to C terminus V_{H}2-TCRβ-C_{H}2-C_{H}3; and the second light chain polypeptide comprises from N to C terminus V_{L}2-TCRα. The use of TCRα and TCRβ constant domain in the bispecific antibody enables correct association of the light chains and their cognate heavy chain, leading to higher yield of desired bispecific antibody against LILRB4 and CD3 (see, e.g., WO2019057122A1). In certain embodiments, the TCRα domain has an amino acid sequence of SEQ ID NO: 89, the TCRβ domain has an amino acid sequence of SEQ ID NO: 90.

Alternatively, as illustrated in the 4ab-3 embodiment in FIG. 28A, the first heavy chain polypeptide comprises from N to C terminus V_{H}1-TCRβ-C_{H}2-C_{H}3; the first light chain polypeptide comprises from N to C terminus V_{L}1-TCRα; the second heavy chain polypeptide comprises from N to C terminus V_{H}2-C_{H1}-C_{H}2-C_{H}3; and the second light chain polypeptide comprises from N to C terminus V_{L}2-C_{L}.

As illustrated in FIG. 28A, the stem of the Y shaped antibody comprises a Fc region consisting of the second and the third constant domains (C_{H}2 and C_{H}3) of the first and second heavy chain polypeptides bound together via disulfide bonding. In certain embodiments, the Fc region is engineered with knobs in holes (KiH) technology (Ridgway J B et al, Protein Eng (1996) 9:617-21; Atwell S et al, J Mol Biol (1997) 270:26-35; Merchant et al, Nature Biotech (1998) 16, 677-681), which prevents homodimerization of the heavy chain polypeptides. In short, each of the constant region of the two heavy chain polypeptides is mutated to create either a knob or a hole, which pairs to promote heterodimerization. The design of the LILRB4/CD3 bispecific antibody using KiH technology enable heterodimerization of the heavy chains and correct association of the light chains and their cognate heavy chain, leading to higher yield of desired bispecific antibody against LILRB4 and CD3.

In certain embodiments, the configuration of the bispecific antibody comprises two copies of the antigen binding sites against LILRB4 and a single copy of the antigen binding site against CD3. This configuration is referred to as 2+1 and illustrated in FIG. 28A. In such configuration, the LILRB4/CD3 bispecific antibody comprises two pairs of heavy chain/light chain polypeptides, which forms a first antigen-binding region and a second antigen-binding region that bind to LILRB4 and CD3, respectively. Distinctive from the 1+1 configuration, in the 2+1 configuration, one heavy chain polypeptide comprises a third heavy chain variable domain (V_{H}3) cognate to a third light variable domain in a third light chain polypeptide, forming a third antigen-binding region binding to LILRB4.

Referring to the embodiment 44-3ab in FIG. 28A, in an exemplary embodiment of the invention, the LILRB4/CD3 bispecific antibody, which is Y shaped and comprises two arms, comprises a first heavy chain polypeptide, a first light chain polypeptide, a second heavy chain polypeptide, a second light chain polypeptide and a third light chain polypeptide. The first heavy chain polypeptide comprises from N to C terminus V_{H}3-C_{H}1-L-V_{H}1-C_{H}1-C_{H}2-C_{H}3, wherein L is a linker (e.g., (G₄S)₂); the first light chain polypeptide comprises from N to C terminus V_{L}1-C_{L}; the second heavy chain polypeptide comprises from N to C terminus V_{H}2-TCRβ-C_{H}2-C_{H}3; and the second light chain polypeptide comprises from N to C terminus V_{L}2-TCRα; the third light chain polypeptide comprises from N to C terminus V_{L}3-C_{L}. On one arm of the antibody, the V_{H}1 and V_{L}1 form a first antigen-binding site against LILRB4, while the V_{H}3 and V_{L}3 form a second antigen-binding site against LILRB4. On the other arm of the antibody, the V_{H}2 and V_{L}2 form an antigen-binding site capable of specifically binding to CD3. In certain embodiment, the third light chain polypeptide V_{L}3-C_{L} may be identical to the first light chain polypeptide V_{L}1-C_{L}.

Referring to the embodiment 4ab4ab-3 in FIG. 28A, in an exemplary embodiment of the invention, the LILRB4/CD3 bispecific antibody comprises a first heavy chain polypeptide, a first light chain polypeptide, a second heavy chain polypeptide, a second light chain polypeptide and a third light chain polypeptide. The first heavy chain polypeptide comprises from N to C terminus V_{H}3-TCRβ-L-V_{H}1-TCRβ-C_{H}2-C_{H}3, wherein L is a linker (e.g., (G₄S)₂); the first light chain polypeptide comprises from N to C terminus V_{L}1-TCRα; the second heavy chain polypeptide comprises from N to C terminus V_{H}2-C_{H}1-C_{H}2-C_{H}3; and the second light chain polypeptide comprises from N to C terminus V_{L}2-C_{L}; the third light chain polypeptide comprises from N to C terminus V_{L}3-TCRα. On one arm of the antibody, the V_{H}1 and V_{L}1 form a first antigen-binding site against LILRB4, while the V_{H}3 and V_{L}3 form a second antigen-binding site against LILRB4. On the other arm of the antibody, the V_{H}2 and V_{L}2 form an antigen-binding site capable of specifically binding to CD3. In certain embodiment, the third light chain polypeptide V_{L}3-TCRα may be identical to the first light chain polypeptide V_{L}1-TCRα.

Referring to the embodiment 43ab-4 in FIG. 28A, in an exemplary embodiment of the invention, the LILRB4/CD3 bispecific antibody comprises a first heavy chain polypeptide, a first light chain polypeptide, a second heavy chain polypeptide, a second light chain polypeptide and a third light chain polypeptide. The first heavy chain polypeptide comprises from N to C terminus V_{H}3-C_{H}1-L-V_{H}2-TCRβ-C_{H}2-C_{H}3, wherein L is a linker (e.g., (G₄S)₂); the first light chain polypeptide comprises from N to C terminus V_{L}2-TCRα; the second heavy chain polypeptide comprises from N to C terminus V_{H}1-C_{H}1-C_{H}2-C_{H}3; and the second light chain polypeptide comprises from N to C terminus V_{L}1-C_{L}; the third light chain polypeptide comprises from N to C terminus V_{L}3-C_{L}. On one arm of the antibody, the V_{H}1 and V_{L}1 form a first antigen-binding site against LILRB4. On the other arm of the antibody, the V_{H}3 and V_{L}3 form a second antigen-binding site against LILRB4, while the V_{H}2 and V_{L}2 form an antigen-binding site capable of specifically binding to CD3. In certain embodiment, the third light chain polypeptide V_{L}3-C_{L} may be identical to the first light chain polypeptide V_{L}1-C_{L}.

Referring to the embodiment 4ab3-4ab in FIG. 28A, in an exemplary embodiment of the invention, the LILRB4/CD3 bispecific antibody comprises a first heavy chain polypeptide, a first light chain polypeptide, a second heavy chain polypeptide, a second light chain polypeptide and a third light chain polypeptide. The first heavy chain polypeptide comprises from N to C terminus V_{H}3-TCRβ-L-V_{H}2-C_{H1}-C_{H}2-C_{H}3, wherein L is a linker (e.g., (G₄S)₂); the first light chain polypeptide comprises from N to C terminus V_{L}2-C_{L}; the second heavy chain polypeptide comprises from N to C terminus V_{H}1-TCRβ-C_{H}2-C_{H}3; and the second light chain polypeptide comprises from N to C terminus V_{L}1-TCRα; the third light chain polypeptide comprises from N to C terminus V_{L}3-TCRα. On one arm of the antibody, the V_{H}1 and V_{L}1 form a first antigen-binding site against LILRB4. On the other arm of the antibody, the V_{H}3 and V_{L}3 form a second antigen-binding site against LILRB4, while the V_{H}2 and V_{L}2 form an antigen-binding site against CD3. In certain embodiment, the third light chain polypeptide V_{L}3-TCRα may be identical to the first light chain polypeptide V_{L}1-TCRα.

In certain embodiments, the antigen-binding site directed to CD3 is generated based on the anti-CD3 antibodies known in the art, e.g., the anti-CD3 antibodies described in WO2019057099, SP34 (Pessano et al EMBO J (1985) 4, 337-334), OKT3 (Ortho, Raritan, NJ; Van Wauwe et al, J Immunol (1984) 133, 129-32), M291 (Protein Design Laboratories, Fremont, CA), BC3 (Fred Hutchinson Cancer Research Center, Seattle, WA), TR66 (Novus Biologicals, Centennial, CO) and BMA030 (Walker C et al., Eur J Immunol. (1907) 17:1611-8).

In certain embodiments, the LILRB4/CD3 bispecific antibody can be engineered to improve homogeneity and manufacturability. In some embodiments, as illustrated in FIG. 28B, the TCRα can be mutated at S91A of SEQ ID NO: 89 to remove 0-glycan modification site. In some embodiment, a Q1E mutation can be made at the N-terminal of the heavy chain or light polypeptide to prevent N-terminal pyro-Q formation.

In certain embodiments, anti-LILRB4 bispecific antibodies could be constructed in many other ways as reviewed by Konterman et al 2017, 9 182-212. In particular anti-LILRB4 bispecific antibodies could be constructed as covalent antibody conjugates, asymmetric F(ab′)2, CovX-bodies, mouse/rat chimeric IgGs, κλ-bodies with common heavy chains, tandem single-chain variable domains (scFv), BiTEs, triplebodies, diabodies, tandem domain antibodies, scFv fusions with C_{H1}/CL domains, Fab-scFv bibodies or tribodies, Fab-Fv fusion, Fab-single domain antibody (sdAb)/VHH fusions, orthogonal Fab-Fab, scFv2-albumin/toxin fusions, single-chain diabody-albumin/toxin fusion, tandem scFv albumin/toxin fusions, dock-and-lock (DNL) Fab₃, DNL-Fab₂-scFv, DNL-Fab-IgG fusions, ImmTAC TCR-scFv fusions, IgG with different heavy chains and different or common light chains, IgG-scFv fusions to the heavy or light chain N or C terminus, IgG single-chain Fab (scFab) fusions to the heavy or light chain N- or C-terminus, single-chain IgG (scIgG) with scFv fusions, dual-variable domain (DVD) bispecific antibodies, asymmetric scFv-Fc, tandem-scFv-Fc fusions, dual affinity re-tarting antibodies (DART) with or without Fc fusion, asymmetric Fab-scFv-Fc fusions, scFv-CH3 fusions, TriFabs, IgG tandem scFv fusions, IgG-crossFab fusions, tandem Fab-IgG fusions with orthogonal Fabs, didiabody-Fc fusion, single-chain diabody Fc-fusion, Fab-scFv-Fc fusions, scFv₄-Fc fusion, scFv2-Fcab, Di-diabody, single-chain diabody CH3 fusion, IgE/M CH2 fusions, F(ab′)₂ fusions, CH1/CK fusions, two-in-one dual action Fabs (DAF), or DutaMab, DNL-Fab₂-IgG fusion.

Heterodimizeration of heavy chains for bispecific antibodies which contain Fc domains can be accomplished by a number of means including but not limited to knob-in-holes (Ridgway et al PEDS 1996; Atwell et al J Mol Biol 1997; Merchant et al Nat Biotechnol 1998), HA-TF mutations (Moore et al Mabs 2011), ZW1 (Von Kreudenstein et al MAbs 2013), CH3 charge pairs (Gunasekaran et al J Biol Chem 2010), IgG1 hinge/CH3 charge pairs (Strop et al J Mol Biol 2012), IgG2 hinge/CH3 charge pairs (Strop et al J Mol Biol 2012), EW-RVT mutations with or without an engineered disulfide (Choi et al Mol Cancer Ther 2013; Choi et al Mol Immunol 2015), biclonic (Geuijen et al J Clinical Onc 2014), DuoBody (Labrijn et al Proc Natl Acad Sci USA 2013), SEEDbody IgG/A chimera (David et al Protein Eng Des Sel 2010), BEAT (Moretti et al BMC Proceedings 2013), 7.8.60 or 29.8.34 (Leaver-Fey et al Structure 2016). Correct pairing of different light chains can be accomplished by a variety of methods including, but not limited to CrossMab (Schaefer et al Cancer Cell 2011), orthogonal Fab (Lewis et al Nat Biotechnol 2014), T-cell receptor fusions (Wu et al MAbs 2015), CR3 (Golay et al J Immunol 2016), MUT4 (Golay et al J Immunol 2016), DuetMab (Mazor et al MAbs 2015, 7, 377-89; Mazor et al MAbs 2015, 7, 461-669).

In certain embodiments, bispecific antibodies could be targeted to LILRB4 and another target including, but not limited to CD3, CD2, CD16a, NKp46, CD137, OX40, PD-1, PD-L1, CD40, CTLA4, LAG3, TIM3, CD47.

### Conjugates

In some embodiments, the anti-LILRB4 antibodies and antigen-binding fragments thereof further comprise a conjugate moiety. The conjugate moiety can be linked to the antibodies and antigen-binding fragments thereof. A conjugate moiety is a proteinaceous or non-proteinaceous moiety that can be attached to the antibody or antigen-binding fragment thereof. It is contemplated that a variety of conjugate moieties may be linked to the antibodies or antigen-binding fragments provided herein (see, for example, “Conjugate Vaccines”, Contributions to Microbiology and Immunology, J. M. Cruse and R. E. Lewis, Jr. (eds.), Carger Press, New York, (1989)). These conjugate moieties may be linked to the antibodies or antigen-binding fragments by covalent binding, affinity binding, intercalation, coordinate binding, complexation, association, blending, or addition, among other methods.

In certain embodiments, the antibodies and antigen-binding fragments disclosed herein may be engineered to contain specific sites outside the epitope binding portion that may be utilized for binding to one or more conjugate moieties. For example, such a site may include one or more reactive amino acid residues, such as for example cysteine or histidine residues, to facilitate covalent linkage to a conjugate moiety.

In certain embodiments, the antibodies may be linked to a conjugate moiety indirectly, or through another conjugate moiety. For example, the antibody or antigen-binding fragments may be conjugated to biotin, then indirectly conjugated to a second conjugate that is conjugated to avidin. The conjugate can be a clearance-modifying agent, a toxin (e.g., a chemotherapeutic agent), a detectable label (e.g., a radioactive isotope, a lanthanide, a luminescent label, a fluorescent label, or an enzyme-substrate label), or purification moiety.

A “toxin” can be any agent that is detrimental to cells or that can damage or kill cells. Examples of toxin include, without limitation, taxol, cytochalasin B, gramicidin D, ethidium bromide, emetine, mitomycin, etoposide, tenoposide, vincristine, monomethyl auristatin E (MMAE), monomethyl auristatin F (MMAF), mertansine, emtansine, DM1, maytansinoid DM1, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), anti-mitotic agents (e.g., vincristine and vinblastine), a topoisomerase inhibitor, and a tubulin-binders.

Examples of detectable label may include a fluorescent labels (e.g. fluorescein, rhodamine, dansyl, phycoerythrin, or Texas Red), enzyme-substrate labels (e.g. horseradish peroxidase, alkaline phosphatase, luceriferases, glucoamylase, lysozyme, saccharide oxidases or β-D-galactosidase), radioisotopes (e.g. ¹²³I, ¹²⁴I, ¹²⁵I, ¹³¹I, ³⁵S, ³H, ¹¹¹In, ¹¹²In ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P, other lanthanides), luminescent labels, chromophoric moiety, digoxigenin, biotin/avidin, a DNA molecule or gold for detection.

In certain embodiments, the conjugate moiety can be a clearance-modifying agent which helps increase half-life of the antibody. Illustrative examples include water-soluble polymers, such as PEG, carboxymethylcellulose, dextran, polyvinyl alcohol, polyvinyl pyrrolidone, copolymers of ethylene glycol/propylene glycol, and the like. The polymer may be of any molecular weight, and may be branched or unbranched. The number of polymers attached to the antibody may vary, and if more than one polymers are attached, they can be the same or different molecules.

In certain embodiments, the conjugate moiety can be a purification moiety such as a magnetic bead.

In certain embodiments, the antibodies and antigen-binding fragments thereof provided herein is used for a base for a conjugate.

### Polynucleotides and Recombinant Methods

The present disclosure provides isolated polynucleotides that encode the anti-LILRB4 antibodies and antigen-binding fragments thereof. In certain embodiments, the isolated polynucleotides comprise one or more nucleotide sequences as shown in SEQ ID NOs: 2, 4, and 35, which encodes the variable region of the exemplary antibodies provided herein. DNA encoding the monoclonal antibody is readily isolated and sequenced using conventional procedures (e.g., by using oligonucleotide probes that are capable of binding specifically to genes encoding the heavy and light chains of the antibody). The encoding DNA may also be obtained by synthetic methods.

The isolated polynucleotide that encodes the anti-LILRB4 antibodies and antigen-binding fragments can be inserted into a vector for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g. SV40, CMV, EF-1α), and a transcription termination sequence.

The present disclosure provides vectors (e.g., expression vectors) containing the nucleic acid sequence provided herein encoding the antibodies or antigen-binding fragments, at least one promoter (e.g., SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the antibody or antigen-binding fragment can be introduced to a host cell for cloning or gene expression. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. lichenformis, Pseudomonas such as P. aeruginosa, and Streptomyces.

In addition to prokaryotes, eukaryotic microbes such as filamentous fungi or yeast are suitable cloning or expression hosts for anti-LILRB4 antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies or antigen-fragment provided here are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. (1977) 36:59); baby hamster kidney cells (BHK, ATCC CCL 10); Chinese Hamster Ovary cells (CHO), CHO cells deficient in dihydrofolate reductase (DHFR) activity, CHO-DHFR (Urlaub et al., Proc. Natl. Acad. Sci. USA (1980) 77:4216); mouse sertoli cells (TM4, Mather, Biol. Reprod. (1980) 23:243-251); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. (1982) 383:44-68); MRC 5 cells; FS4 cells; and a human hepatoma line (Hep G2). In some preferable embodiments, the host cell is 293F cell.

Host cells are transformed with the above-described expression or cloning vectors for anti-LILRB4 antibody production and cultured in conventional nutrient media modified as appropriate for inducing promoters, selecting transformants, or amplifying the genes encoding the desired sequences. In another embodiment, the antibody may be produced by homologous recombination known in the art.

The host cells used to produce the antibodies or antigen-binding fragments provided herein may be cultured in a variety of media. Commercially available media such as Ham's F10 (Sigma), Minimal Essential Medium (MEM) (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. (1980) 102:255, U.S. Pat. Nos. 4,767,704; 4,657,866; 4,927,762; 4,560,655; or 5,122,469; WO 90/03430; WO 87/00195; or U.S. Pat. Re. 30,985 maybe used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology (1992) 10:163-167 describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The anti-LILRB4 antibodies and antigen-binding fragments thereof prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the antibody and antigen-binding fragment thereof. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. (1983) 62:1-13). Protein G is recommended for all mouse isotypes and for human gamma3 (Guss et al., EMBO J. (1986)5:1567-75). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### Purification

In certain embodiments, the antibodies of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the number of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### III. PHARMACEUTICAL COMPOSITION

The present disclosure further provides pharmaceutical compositions comprising the anti-LILRB4 antibodies or antigen-binding fragments thereof and one or more pharmaceutically acceptable carriers.

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising an antibody or antigen-binding fragment and conjugates as provided herein decreases oxidation of the antibody or antigen-binding fragment. This reduction in oxidation prevents or reduces loss of binding affinity, thereby improving antibody stability and maximizing shelf-life. Therefore, in certain embodiments compositions are provided that comprise one or more antibodies or antigen-binding fragments as disclosed herein and one or more antioxidants such as methionine. Further provided are methods for preventing oxidation of, extending the shelf-life of, and/or improving the efficacy of an antibody or antigen-binding fragment as provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agents. The solvent may contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the anti-LILRB4 antibody or antigen-binding fragment thereof or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

### IV. METHODS OF USE OF ANTI-LILRB4 ANTIBODIES

The present disclosure also provides therapeutic methods comprising: administering a therapeutically effective amount of the antibody or antigen-binding fragment as provided herein to a subject in need thereof, thereby treating or preventing a LILRB4-related condition or a disorder. In some embodiment, the LILRB4-related condition or a disorder is cancer, autoimmune disease, inflammatory disease, or infectious disease.

Examples of cancer can be generally categorized into solid tumors and hematologic malignancies. Solid tumors include but are not limited to, non-small cell lung cancer (squamous/non-squamous), small cell lung cancer, renal cell cancer, colorectal cancer, colon cancer, ovarian cancer, breast cancer (including basal breast carcinoma, ductal carcinoma and lobular breast carcinoma), pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, melanoma, multiple myeloma, mycoses fungoides, Merkel cell cancer, hepatocellular carcinoma (HCC), fibrosarcoma, myxosarcoma, liposarcoma, chondrosarcoma, osteogenic sarcoma, and other sarcomas, synovioma/synovial sarcoma, mesothelioma, Ewing's sarcoma, leiomyosarcoma, rhabdomyosarcoma, basal cell carcinoma, adenocarcinoma, sweat gland carcinoma, medullary thyroid carcinoma, papillary thyroid carcinoma, pheochromocytoma, sebaceous gland carcinoma, papillary carcinoma, papillary adenocarcinomas, medullary carcinoma, bronchogenic carcinoma, hepatoma, bile duct carcinoma, choriocarcinoma, Wilms' tumor, cervical cancer, testicular tumor, seminoma, mast cell derived tumors, EBV-positive and -negative PTLD, nasopharyngeal carcinoma, spinal axis tumor, brain stem glioma, astrocytoma, medulloblastoma, craniopharyogioma, ependymoma, pinealoma, hemangioblastoma, acoustic neuroma, oligodendroglioma, meningioma, melanoma, neuroblastoma and retinoblastoma.

Solid tumors are characterized by multiple biologic hallmarks including sustaining proliferative signaling, evading growth suppressors, resisting cell death, enabling replicative immortality, inducing angiogenesis, activating invasion and metastasis, tumor promoting inflammation, avoiding immune destruction, genomic instability and mutation, and deregulating cellular energetics. Treatment efforts have evolved from cytotoxic chemotherapies targeting rapidly dividing cells to small molecules inhibiting select signaling pathways to monoclonal antibodies targeting surface proteins. More recently the concept of cancer immunotherapy to reinvigorate endogenous immunity or cellular therapies utilizing synthetic immunity have shown promise. Despite these advances, most patients with advanced solid tumors still do not survive long-term. The use of immune checkpoint inhibitors such as anti-CTLA-4 or anti-PD-1/PD-L1 have led to long-term progression-free and overall survival in a minority of patients.

Newer immunotherapy approaches targeting different aspects of immune biology and different tumor-infiltrating cells are needed to improve outcomes, such as those targeting LILRB4 as an inhibitory receptor expressed on subset of myeloid cells, including myeloid derived suppressor cells (MDSC). These myeloid cells are described functionally as myeloid derived suppressive cells because their immune suppressive/anti-inflammatory phenotype can inhibit the activation, proliferation and cytotoxic activity of tumor antigen-specific T cells.

In some embodiments, depleting MDSC may revert the suppressive effect on tumor antigen-specific T cells for solid tumor treatment.

In some embodiments, blocking LILRB4 on myeloid cells could also unlock its inhibitory effects on antigen presentation cells (APCs), including dendritic cells or myeloid leukemia cells which express LILRB4. Increased antigen presenting activity can be observed by certain cytokines produced APCs, and can leads to T cell activation, cytotoxicity and T cell cytokine production.

Hematologic malignancies include but are not limited to acute lymphocytic/lymphoblastic leukemia (ALL), acute myeloid leukemia (AML), B-cell leukemia, blastic plasmacytoid dendritic cell neoplasm (BPDCN), chronic lymphoblastic leukemia (CLL), chronic lymphocytic leukemia (CLL), chronic myeloid leukemia (CML), chronic myelomonocytic leukemia (CMML), classical Hodgkin lymphoma (CHL), diffuse large B-cell lymphoma (DLBCL), extranodal NK/T-cell lymphoma, hairy cell leukemia, heavy chain disease, HHV8-associated primary effusion lymphoma, lymphoid malignancy, multiple myeloma (MM), myelodysplasia, myelodysplastic syndrome (MDS), non-Hodgkin's lymphoma, plasmablastic lymphoma, pre-B acute lymphocytic leukemia (Pre-B ALL), primary CNS lymphoma, primary mediastinal large B-cell lymphoma, T-cell/histiocyte-rich B-cell lymphoma, myeloproliferative neoplasms, and Waldenstrom's macroglobulinemia.

Autoimmune or inflammatory diseases include, but are not limited to, Acquired Immunodeficiency Syndrome (AIDS, which is a viral disease with an autoimmune component), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, CREST syndrome, Crohn's disease, Degos' disease, dermatomyositis-juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma, systemic scleroderma, progressive systemic sclerosis (PSS), systemic sclerosis (SS), Sjogren's syndrome, stiff-man syndrome, systemic lupus erythematosus (SLE), Takayasu arteritis, temporal arteritis/giant cell arteritis, inflammatory bowel disease (IBD), ulcerative colitis, Cohn's disease, intestinal mucosal inflammation, wasting disease associated with colitis, uveitis, vitiligo and Wegener's granulomatosis, Alzheimer's disease, asthma, atopic allergy, allergy, atherosclerosis, bronchial asthma, eczema, glomerulonephritis, graft vs. host disease, hemolytic anemias, osteoarthritis, sepsis, stroke, transplantation of tissue and organs, vasculitis, diabetic retinopathy, ventilator induced lung injury, viral infections, autoimmune diabetes and the like. Inflammatory disorders, include, for example, chronic and acute inflammatory disorders.

Infectious disease include, but are not limited to, fungus infection, parasite/protozoan infection or chronic viral infection, for example, malaria, coccidioiodmycosis immitis, histoplasmosis, onychomycosis, aspergillosis, blastomycosis, candidiasis albicans, paracoccidiodomycosis, microsporidiosis, Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, Trichuriasis, Trypanosomiasis, helminth infection, infection of hepatitis B (HBV), hepatitis C (HCV), herpes virus, Epstein-Barr virus, HIV, cytomegalovirus, herpes simplex virus type I, herpes simplex virus type II, human papilloma virus, adenovirus, human immunodeficiency virus I, human immunodeficiency virus II, Kaposi West sarcoma associated herpes virus epidemics, thin ring virus (Torquetenovirus), human T lymphotrophic virus I, human T lymphotrophic virus II, varicella zoster, JC virus or BK virus.

The therapeutically effective amount of an antibody or antigen-binding fragment as provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by one of ordinary skill in the art (e.g., physician or veterinarian) as indicated by these and other circumstances or requirements.

In certain embodiments, the antibody or antigen-binding fragment as provided herein may be administered at a therapeutically effective dosage of about 0.0001 mg/kg to about 100 mg/kg. In certain of these embodiments, the antibody or antigen-binding fragment is administered at a dosage of about 50 mg/kg or less, and in certain of these embodiments the dosage is 10 mg/kg or less, 5 mg/kg or less, 3 mg/kg or less, 1 mg/kg or less, 0.5 mg/kg or less, or 0.1 mg/kg or less. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The antibodies and antigen-binding fragments disclosed herein may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes.

In some embodiments, the antibodies or antigen-binding fragments disclosed herein may be administered alone or in combination with one or more additional therapeutic means or agents. For example, the antibodies or antigen-binding fragments disclosed herein may be administered in combination with another therapeutic agent, for example, a chemotherapeutic agent or an anti-cancer drug.

In certain of these embodiments, an antibody or antigen-binding fragment as disclosed herein that is administered in combination with one or more additional therapeutic agents may be administered simultaneously with the one or more additional therapeutic agents, and in certain of these embodiments the antibody or antigen-binding fragment and the additional therapeutic agent(s) may be administered as part of the same pharmaceutical composition. However, an antibody or antigen-binding fragment administered “in combination” with another therapeutic agent does not have to be administered simultaneously with or in the same composition as the agent. An antibody or antigen-binding fragment administered prior to or after another agent is considered to be administered “in combination” with that agent as the phrase is used herein, even if the antibody or antigen-binding fragment and second agent are administered via different routes. Where possible, additional therapeutic agents administered in combination with the antibodies or antigen-binding fragments disclosed herein are administered according to the schedule listed in the product information sheet of the additional therapeutic agent, or according to the Prescriber's Digital Reference (available online only at pdr.net) or protocols well known in the art.

Particular agents contemplated for combination therapy with antibodies of the present disclosure include chemotherapy. Chemotherapy may include cytarabine (ara-C) and an anthracycline (most often daunorubicin), high-dose cytarabine alone, all-trans-retinoic acid (ATRA) in addition to induction chemotherapy, usually an anthracycline, histamine dihydrochloride (Ceplene) and interleukin 2 (Proleukin) after the completion of consolidation therapy, gemtuzumab ozogamicin (Mylotarg) for patients aged more than 60 years with relapsed AML who are not candidates for high-dose chemotherapy, clofarabine, as well as targeted therapies, such as kinase inhibitors, farnesyl transferase inhibitors, decitabine, and inhibitors of MDR1 (multidrug-resistance protein), or arsenic trioxide or relapsed acute promyelocytic leukemia (APL).

In certain embodiments, the agents for combination therapy are one or more drugs selected from the group consisting of a topoisomerase inhibitor, an anthracycline topoisomerase inhibitor, an anthracycline, a daunorubicin, a nucleoside metabolic inhibitor, a cytarabine, a hypomethylating agent, a low dose cytarabine (LDAC), a combination of daunorubicin and cytarabine, a daunorubicin and cytarabine liposome for injection, Vyxeos®, an azacytidine/azacitidine, Vidaza®, a decitabine, an all-trans-retinoic acid (ATRA), an arsenic, an arsenic trioxide, a histamine dihydrochloride, Ceplene®, an interleukin-2, an aldesleukin, Proleukin®, a gemtuzumab ozogamicin, Mylotarg®, an FLT-3 inhibitor, a midostaurin, Rydapt®, a clofarabine, a farnesyl transferase inhibitor, a decitabine, an IDH1 inhibitor, an ivosidenib, Tibsovo®, an IDH2 inhibitor, an enasidenib, Idhifa®, a smoothened (SMO) inhibitor, a glasdegib, an arginase inhibitor, an IDO inhibitor, an epacadostat, a BCL-2 inhibitor, a venetoclax, Venclexta®, a platinum complex derivative, oxaliplatin, a kinase inhibitor, a tyrosine kinase inhibitor, a PI3 kinase inhibitor, a BTK inhibitor, an ibrutinib, IMBRUVICA®, an acalabrutinib, CALQUENCE®, a zanubrutinib, a PD-1 antibody, a PD-L1 antibody, a CTLA-4 antibody, a LAG3 antibody, an ICOS antibody, a TIGIT antibody, a TIM13 antibody, a CD40 antibody, a 4-1BB antibody, a CD47 antibody, a SIRP1α antibody or fusions protein, an antagonist of E-selectin, an antibody binding to a tumor antigen, an antibody binding to a T-cell surface marker, an antibody binding to a myeloid cell or NK cell surface marker, an alkylating agent, a nitrosourea agent, an antimetabolite, an antitumor antibiotic, an alkaloid derived from a plant, a hormone therapy medicine, a hormone antagonist, an aromatase inhibitor, and a P-glycoprotein inhibitor.

In certain embodiments, the LILRB4-related condition or a disorder is acute myeloid leukemia (AML), Acute Myelomonocytic Leukemia (FAB M4) subtype and Acute Monoblastic/Monocytic Leukemia (FAB M5) subtype.

In certain embodiments, AML that is resistant or refractory to standard of care treatment such as venetoclax and azacytidine/azacitidine.

AML is an aggressive malignancy with poor prognosis. In the World Health Organization (WHO) classification, acute myelomonocytic leukemia (M4 in the French-American-British [FAB] classification) and acute monoblastic/monocytic leukemia (M5 in the FAB classification) are subtypes of AML, Not Otherwise Specified (AML, NOS).

Acute myelomonocytic leukemia in the WHO system (FAB M4) has blasts consisting of myeloblasts, monoblasts, and promonocytes (total of ≥20%), and ≥20% to 79% of monocytic lineage. Acute monoblastic/monocytic leukemia (FAB M5) has monoblasts/promonocytes≥20%, and ≥80% of marrow cells with monocytic features. Acute myelomonocytic leukemia (M4) and acute monocytic leukemia (M5) account for approximately 20% and 10% of all cases of AML, respectively (Ganzel et al., 2016). AML patients with a significant monocytic component are more likely to have evidence of extramedullary disease (Ganzel et al., 2016) and hyperleukocytosis (defined as ≥100×10³/L white blood cells in the peripheral blood) (Rollig and Ehninger, 2015).

LILRB4 is expressed on AML cells with monocytic differentiation (Deng et al., 2018; Dobrowolska et al., 2013) and CMML (Chien et al., 2019). The expression level of LILRB4 may be equivalent to normal monocytes or up to 10-fold higher on AML blasts with monocytic differentiation (Deng et al., 2018). Moreover, both functional and immunophenotypic studies suggest that LILRB4 is expressed by leukemic stem cells from monocytic AML (Deng et al., 2018).

In 2008, AZA was approved by the European Medicines Agency (EMA) for the treatment of AML patients with 20% to 30% bone marrow (BM) blasts, older than 64 years and who are ineligible for hematopoietic stem cell transplant (HSCT). Hematologists at specialized centers started treating AML patients with >30% BM blasts with AZA as early as 2007, indicating that the physicians were convinced they were doing the best for their patients. This assumption was based on the significant improvement of overall survival (OS) obtained in the AZA-MDS-001 trial and the Cancer and Leukemia Group B protocols, in which 32% and 38% of the trial population had AML with 20% to 30% BM blasts, respectively.

In 2010, the international phase 3 randomized AZA-AML-001 clinical trial testing AZA versus conventional care regimens (CCR) (intensive chemotherapy, low-dose cytarabine or best supportive care as preselected by the treating physician) in AML patients older than 65 years with newly-diagnosed AML, >30% BM blasts and ≤15×10⁹/L white blood cell (WBC) count was initiated. In this trial, a clinically meaningful improvement in OS for AZA versus CCR (10.4 vs. 6.5 months; p=0.1009) was reported. Additionally, the overall response (complete remission [CR]+ complete remission with incomplete blood count recovery [CRi]) rates were comparable in the AZA (27.8%) and CCR (25.1%) arms (P=0.5384) and the EMA approval of AZA was expanded on 30 Oct. 2015 to include AML patients with >30% BM blasts.

In certain embodiments, the LILRB4-related condition or a disorder is Chronic Myelomonocytic Leukemia (CMML).

CMML diagnostic classification is based on clinical examination, morphology, cytogenetics, and, whenever possible, flow cytometry and molecular biology should be integrated to classify patients according to WHO 2016 categories. WHO classification (Arber et al., 2016) includes CMML-0 for cases with <2% blasts in peripheral blood and <5% blasts in BM; CMML-1 for cases with 2% to 4% blasts in peripheral blood and/or 5% to 9% blasts in BM; and CMML-2 for cases with 5% to 19% blasts in peripheral blood, 10% to 19% in BM, and/or when Auer rods are present.

The distinction between “dysplastic” CMML and “proliferative” CMML initially proposed by the FAB classification based on a WBC cutoff of 13×10⁹/L remains useful, as their clinical features differ (cytopenia vs. organomegaly, high WBC, and constitutional symptoms), and consequently, their clinical management.

Extramedullary leukemia, apart from splenomegaly and hepatomegaly, mainly includes specific serous effusions (pleural and less often pericardial or peritoneal) and specific cutaneous infiltration, all associated with worse prognosis.

VIDAZA® (azacitidine) is a nucleoside metabolic inhibitor indicated for the treatment of patients with the following FAB myelodysplastic syndrome (MDS) subtypes: Refractory anemia or refractory anemia with ringed sideroblasts (if accompanied by neutropenia or thrombocytopenia or requiring transfusions), refractory anemia with excess blasts, refractory anemia with excess blasts in transformation, and chronic myelomonocytic leukemia (CMML).

The present disclosure further provides methods of using the anti-LILRB4 antibodies or antigen-binding fragments thereof to detect presence or amount of LILRB4 in a sample, comprising contacting the sample with the antibody or antigen-binding fragment thereof, and determining the presence or the amount of LILRB4 in the sample. The method of detecting LILRB4 using an anti-LILRB4 antibody includes, without limitation, ELISA, Western-blot, flow cytometry and FACS.

In some embodiments, the present disclosure provides methods of diagnosing a LILRB4 related disease or condition in a subject, comprising: a) contacting a sample obtained from the subject with the antibody or antigen-binding fragment thereof provided herein; b) determining presence or amount of LILRB4 in the sample; and c) correlating the existence of the LILRB4 to the LILRB4 related disease or condition in the subject.

In some embodiments, the present disclosure provides kits comprising the antibody or antigen-binding fragment thereof provided herein, optionally conjugated with a detectable moiety. The kits may be useful in detection of LILRB4 or diagnosis of LILRB4 related disease.

In some embodiments, the present disclosure also provides use of the antibody or antigen-binding fragment thereof provided herein in the manufacture of a medicament for treating a LILRB4 related disease or condition in a subject, in the manufacture of a diagnostic reagent for diagnosing a LILRB4 related disease or condition.

### V. CHIMERIC ANTIGEN RECEPTORS

The present disclosure in another aspect provides a chimeric antigen receptor (CAR) protein that binds LILRB4 (LILRB4 CAR protein). In certain embodiments, the CAR protein comprises an antigen recognition region, i.e., an antibody or antigen-binding fragment that recognizes LILRB4 as described herein, and other membrane and intracellular components. In some embodiments, the LILRB4 CAR protein comprises a LILRB4 antigen recognition region, a transmembrane domain and an intracellular co-stimulatory signal domain. In certain embodiments, the single chain LILRB4 CAR protein also comprises a leader peptide, a spacer region and an intracellular T cell signaling domain.

In certain embodiments, the antigen recognition region comprises multiple polypeptide chains.

In some embodiments, the CAR protein comprises a first polypeptide including an antibody heavy chain variable domain and a polypeptide including an antibody light chain variable domain, wherein the first or the second polypeptide further includes a transmembrane domain, and wherein the antibody heavy chain variable domain and the antibody light chain variable domain together form an antigen recognition region.

In some embodiments, the CAR protein comprises a first polypeptide including an antibody heavy chain variable domain and a second polypeptide including an antibody light chain variable domain and an antibody light chain constant domain, wherein the first polypeptide further includes a transmembrane domain, and wherein the antibody heavy chain variable domain, the antibody light chain variable domain and the antibody light chain constant domain together form an antigen recognition region. In some embodiments, the first portion further includes an intracellular co-stimulatory signaling domain and a CD3C intracellular T cell signaling domain.

In some embodiments, the CAR protein comprises a first polypeptide including an antibody heavy chain variable domain and an antibody heavy chain constant domain, and a second polypeptide including an antibody light chain variable domain, wherein the first polypeptide further includes a transmembrane domain, and wherein the antibody heavy chain variable domain, the antibody heavy chain constant domain, and the antibody light chain variable domain together form an antigen recognition region. In some embodiments, the first portion further includes an intracellular co-stimulatory signaling domain and a CD3C intracellular T cell signaling domain.

In some embodiments, the CAR protein comprises a first polypeptide including an antibody heavy chain variable domain and a second polypeptide including an antibody light chain variable domain, wherein the second polypeptide further includes a transmembrane domain, and wherein the antibody heavy chain variable domain, the antibody light chain variable domain and the antibody light chain constant domain together form an antigen recognition region. In some embodiments, the second portion further includes an intracellular co-stimulatory signaling domain and a CD3C intracellular T cell signaling domain.

In some embodiments, the CAR protein comprises a first polypeptide including an antibody heavy chain variable domain and an antibody heavy chain constant domain, and a second polypeptide including an antibody light chain variable domain, wherein the second polypeptide further includes a transmembrane domain, and wherein the antibody heavy chain variable domain, the antibody heavy chain constant domain, and the antibody light chain variable domain together form an antigen recognition region. In some embodiments, the second portion further includes an intracellular co-stimulatory signaling domain and a CD3ζ intracellular T cell signaling domain.

In certain embodiments, the CAR protein is a single chain polypeptide that comprises an anti-LILRB4 scFv as described herein, i.e., an anti-LILRB4 heavy chain variable domain and an anti-LILRB4 light chain variable domain, which are linked by a linker domain. In one embodiment, the CAR protein includes from the N-terminus to the C-terminus: a leader peptide, an anti-LILRB4 heavy chain variable domain, a linker domain, an anti-LILRB4 light chain variable domain, a hinge region, a transmembrane domain, an intracellular co-stimulatory signal domain. In one embodiment, the CAR protein includes from the N-terminus to the C-terminus: a leader peptide, an anti-LILRB4 light chain variable domain, a linker domain, an anti-LILRB4 heavy chain variable domain, a hinge region, a transmembrane domain, an intracellular co-stimulatory signal domain. In some embodiments, the CAR protein further includes a CD3ζ intracellular T cell signaling domain.

In certain embodiment, the linker domain generally is comprised of helix- and turn-promoting amino acid residues such as alanine, serine and glycine. However, other residues can function as well. In some embodiment, the linker domain is inserted between the VH and VL of the scFv. In some embodiments, the linker domain is between the transmembrane domain and the intracellular co-stimulatory signaling domain. In some embodiments, the linker domain is between the intracellular T cell signaling domain and the intracellular co-stimulatory signaling domain. In some embodiments, the linker domain comprises the sequence GGGGSGGGGSGGGGS (SEQ ID NO: 70).

In some embodiments, the transmembrane domain is a CD8a transmembrane domain which has at least 90%, 95%, 96%, 97%, 98%, 99% or 100% amino acid sequence identity compared to a naturally occurring CD8α transmembrane domain polypeptide (SEQ ID NO: 71). In some embodiments, the CD8α transmembrane domain is encoded by the nucleic acid sequence of SEQ ID NO: 72.

In some embodiments, the transmembrane domain is a CD28 transmembrane domain which has at least 90%, 95%, 96%, 97%, 98%, 99% or 100% amino acid sequence identity compared to a naturally occurring CD28 transmembrane domain polypeptide (SEQ ID NO: 73). In some embodiments, the CD28 transmembrane domain is encoded by the nucleic acid sequence of SEQ ID NO: 74.

The intracellular co-stimulatory signaling domain includes amino acid sequences capable of providing co-stimulatory signaling in response to binding of an antigen to the CAR. In some embodiments, the signaling of the co-stimulatory signaling domain results in the production of cytokines and proliferation of the T cell or NK cell expressing the same. In some embodiments, the intracellular co-stimulatory signaling domain is a CD28 intracellular co-stimulatory signaling domain, a 4-1BB intracellular co-stimulatory signaling domain, an ICOS intracellular co-stimulatory signaling domain, an OX-40 intracellular co-stimulatory signaling domain or any combination thereof. In some embodiments, the CD28 co-stimulating domain has the polypeptide sequence of SEQ ID NO: 75. In some embodiments, the CD28 intracellular co-stimulatory signaling domain is encoded by the nucleic acid sequence of SEQ ID NO: 76. In some embodiments, the 4-1BB intracellular co-stimulatory signaling domain has the polypeptide sequence of SEQ ID NO: 77. In some embodiments, the 4-1BB intracellular co-stimulatory signaling domain is encoded by the nucleic acid sequence of SEQ ID NO: 78.

A “hinge region” as provided herein is a polypeptide connecting the antigen-binding region with the transmembrane domain. In some embodiments, the hinge region connects a heavy chain variable region with the transmembrane domain. In some embodiments, the hinge region connects a heavy chain constant region with the transmembrane domain. In some embodiments, the hinge region connects a light chain variable region with the transmembrane domain. In some embodiments, the hinge region connects a light chain constant region with the transmembrane domain. In some embodiments, the binding affinity of the antigen-binding region to an antigen is increased compared to the absence of the hinge region. In some embodiments, the steric hindrance between an antigen-binding region and an antigen is decreased in the presence of the hinge region. In some embodiments, the hinge region is a CD8α hinge region. In some embodiments, the hinge region is a CD28 hinge region.

In some embodiments, the intracellular T cell signaling domain includes the signaling domain of the zeta (ζ) chain of the human CD3 complex, i.e., a CD3ζ intracellular T cell signaling domain. In some embodiments, the intracellular T cell signaling domain is the protein CD3zIso1 with the amino acid sequence of SEQ ID No: 79. In some embodiments, the intracellular T cell signaling domain is the protein CD3zIso3 with the amino acid sequence of SEQ ID No: 80, encoded by the nucleic acid sequence of SEQ ID NO: 81.

In one example, the CAR protein is a single chain polypeptide that includes from the N-terminus to the C-terminus: a CD8α leader peptide, an anti-LILRB4 scFv, a CD8α hinge region, a CD8α transmembrane domain (or a CD28 transmembrane domain), a 4-1BB intracellular co-stimulatory signaling domain (or a CD28 intracellular co-stimulatory signaling domain, or a CD28 intracellular co-stimulatory signaling domain followed by a 4-1BB intracellular co-stimulatory signaling domain) and a CD3C intracellular T cell signaling domain in one of two isoforms (CD3zIso1 or CD3zIso3).

In certain embodiments, the LILRB4 CAR protein provided herein demonstrates a high affinity to LILRB4. In certain embodiments, the CAR protein provided herein has a binding affinity to LILRB4 (EC₅₀ as measured by ELISA) of less than 1 nM, 0.9 nM, 0.8 nM, 0.7 nM, 0.6 nM, 0.5 nM, 0.4 nM, 0.3 nM, 0.2 nM, 0.1 nM, 0.09 nM, 0.08 nM, 0.07 nM, 0.06 nM or 0.05 nM. For the purposes of this application, ELISA EC₅₀ values may be determined as follows. LILRB-4 extracellular domain protein (with 6 HIS tag at the C-terminus) was produced recombinantly in HEK293 cells and coated onto a high binding 96-well clear plate (Corning-Costar, Fisher Scientific) at 1 μg/ml concentration (100 μl/well) at 4° C. for 14 to 16 hours. The coated plates were washed with PBS, pH 7.4, briefly and blocked with 200 μl/well of 5% non-fat milk in PBS for 2 hours at 37° C. Serial dilutions of the testing monoclonal antibodies (IgGs or scFvs fragments), starting from 10 μg/ml and 3-fold titration down for 12 steps, were added to the 96-well plate for binding by incubating 45 minutes at 37° C. with a cover on the assay plate. Then the plates were washed with PBS containing Tween 20 (0.05% concentration) for 3 times and PBS one time. Secondary antibody of anti-human or anti-rabbit, or other species IgG specific antibodies with HRP conjugate (Jackson ImmunoResearch) was added for incubation at room temperature for 1 hour per manufacturer's suggested dilution. Detection was conducted by adding HRP substrate, TMB (ThermoFisher) for 10 minutes, and stopped by adding 50 μl/well of 2N H₂SO₄. The plates were read for absorbance at 450 nm using a plate reader (SpectraMax M4, Molecular Devices). Data were collected and graphed using a 4-parameter fitting curve with GrapPad Prism 7 software for EC₅₀ calculation.

In another aspect, the present disclosure provides a polynucleotide molecule encoding a CAR protein described herein. In some embodiments, the polynucleotide molecule further comprises a promoter active in eukaryotic cells. In some embodiments, the promoter is the JeT promoter. The JeT promoter is a recombinant promoter with transcriptional activity comparable to a number of strong mammalian promoters. The JeT promoter consists of five key elements: (1) a TATA box; (2) a transcription initiation site (Inr); (3) a CAT consensus sequence in conjunction with (4) a CArG element and finally, (5) four Spl transcription binding sites (GGGCGG) arranged in two tandems (US 2002/0098547 A1). In some embodiments, the polynucleotide molecule is an expression vector. In some embodiment, the vector is generated based on pLVX-EF1alpha-IRES-ZsGreen from Clontech, or pSIN-EF1alpha-IRES-Puromycin or pSIN-EF1alpha. In one example, the polynucleotide molecule of the present disclosure comprises the following elements sequentially: (1) JeT promoter; (2) sequence encoding a CD8-alpha leader; (3) sequence encoding a heavy chain variable region; (4) sequence encoding a linker; (5) sequence encoding a light chain variable region; (6) sequence encoding a CD8 hinge and TM domain; (7) sequence encoding a 4-1BB co-stimulatory domain; (8) sequence encoding a CD3-zeta activation domain. In one example, the elements described above are flanked by 5′ and 3′ homologous arms that facilitate the insertion of the polynucleotide molecule to a target locus, e.g., T cell receptor alpha constant (TRAC) locus.

### VI. ENGINEERED CELLS EXPRESSING ANTI-LILRB4 CAR PROTEIN

In another aspect, the present disclosure provides engineered immune cells which express a CAR protein described herein. The immune cells may be T cells (e.g., regulatory T cells, CD4+ T cells, CD8+ T cells, or gamma-delta T cells), Natural Killer (NK) cells, invariant NK cells, NKT cells, or macrophages. Also provided herein are methods of producing and engineering the immune cells as well as methods of using and administering the cells for adoptive cell therapy, in which case the cells may be autologous or allogeneic. Thus, the engineered immune cells may be used as immunotherapy, such as to target cancer cells.

Expressing the CAR protein allows the engineered immune cells to bind to a target cell, such as a cancer cell, by recognizing an antigen present on the target cell. Upon binding to the target cell, the engineered immune cell becomes activated, then proceed to proliferate and become cytotoxic, eventually destroys the target cell. CAR-T cell immunotherapy has demonstrated success in clinical trials and been approved by U.S. FDA to treat refractory B-cell acute lymphoblastic leukemia and B-cell non-Hodgkin lymphoma (Hartmann J et al., EMBO Mol Med (2017) 9:1183-97). CAR NK cells and CAR macrophages have been developed recently as immunotherapy options in addition to CAR-T cells (Kloess S et al., Transfusion Medicine and Hemotherapy (2019) 46:4-13; Klichinsky M et al., AACR Annual Meeting 2017, Abstract 4575). Therefore, in certain embodiments of the present disclosure, the immune cells that express the CAR protein described herein are T cells, NK cells or macrophages.

The immune cells may be isolated from subjects, particularly human subjects. The immune cells can be obtained from a subject of interest, such as a subject suspected of having a particular disease or condition, a subject suspected of having a predisposition to a particular disease or condition, a subject who is undergoing therapy for a particular disease or condition, a subject who is a healthy volunteer or healthy donor, or from blood bank. Immune cells can be collected from any location in which they reside in the subject including, but not limited to, blood, cord blood, spleen, thymus, lymph nodes, and bone marrow. The isolated immune cells may be used directly, or they can be stored for a period of time, such as by freezing.

The immune cells may be enriched/purified from any tissue where they reside including, but not limited to, blood (including blood collected by blood banks or cord blood banks), spleen, bone marrow, tissues removed and/or exposed during surgical procedures, and tissues obtained via biopsy procedures. Tissues/organs from which the immune cells are enriched, isolated, and/or purified may be isolated from both living and non-living subjects, wherein the non-living subjects are organ donors. In particular embodiments, the immune cells are isolated from blood, such as peripheral blood or cord blood. In some aspects, immune cells isolated from cord blood have enhanced immunomodulation capacity, such as measured by CD4- or CD8-positive T cell suppression. In specific aspects, the immune cells are isolated from pooled blood, particularly pooled cord blood, for enhanced immunomodulation capacity. The pooled blood may be from 2 or more sources, such as 3, 4, 5, 6, 7, 8, 9, 10 or more sources (e.g., donor subjects).

The population of immune cells can be obtained from a subject in need of therapy or suffering from a disease associated with reduced immune cell activity. Thus, the cells will be autologous to the subject in need of therapy. Alternatively, the population of immune cells can be obtained from a donor, preferably a histocompatibility matched donor. The immune cell population can be harvested from the peripheral blood, cord blood, bone marrow, spleen, or any other organ/tissue in which immune cells reside in said subject or donor. The immune cells can be isolated from a pool of subjects and/or donors, such as from pooled cord blood.

When the population of immune cells is obtained from a donor distinct from the subject, the donor is preferably allogeneic, provided the cells obtained are subject-compatible in that they can be introduced into the subject. Allogeneic donor cells may or may not be human-leukocyte-antigen (HLA)-compatible. To be rendered subject-compatible, allogeneic cells can be treated to reduce immunogenicity.

The immune cells can be genetically engineered to express the CARs using suitable methods of modification are known in the art. See, for instance, Sambrook and Ausubel, CURRENT PROTOCOLS IN MOLECULAR BIOLOGY, Greene Publishing Associates and John Wiley & Sons, NY, 1994. In some embodiments, the immune cells comprise one or more nucleic acids introduced via genetic engineering that encode one or more CAR proteins. In certain embodiments, the nucleic acids encoding the CAR proteins are inserted in the genome of the immune cells using gene editing methods, e.g. CRISPR/Cas technology. In one example, the nucleic acids encoding the CAR proteins are inserted at the T-cell receptor alpha constant (TRAC) locus (see, e.g., Eyquem J et al., Nature (2017) 543:113-117).

Also provided are methods for immunotherapy comprising administering an effective amount of the immune cells of the present disclosure. In some embodiments, a medical disease or disorder is treated by transfer of a population of immune cells described herein that elicits an immune response. In certain embodiments, the medical disease or disorder is a cancer. In certain embodiments, the medical disease or disorder is an autoimmune or inflammatory disease.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### Example 1

### Materials and Methods

SEC: The procedure of size exclusion chromatography (SEC) is as follows. Dilute sample to 10.0 mg/mL with mobile phase before SEC analysis if the sample concentration is above 10.0 mg/mL. 100 g of sample was injected into column. The equipment used was the Agilent 1260 HPLC system with a TSKgel G3000SWXL column (7.8×300 mm, 5 μm particle size) and a UV detector (detection wavelength: 280 nm). Mobile phase was 50 mM phosphate buffer with 300 mM Sodium Chloride (pH 6.8±0.1). An isocratic gradient was applied for 20 min at a flow rate of 1.0 mL/min.

Imaged Capillary Isoelectric Focusing (icIEF): 20 μL of the reference standard or sample (diluted to 1.0 mg/mL) was mixed individually with ˜80 μL of a master mixture, which is composed of 0.5 μL of pI 7.40 marker, 0.5 μL of pI 9.77 marker, 1.0 μL of Pharmalyte 3-10, 3.0 μL of Pharmalyte 8-10.5, 35 μL of 1% methyl cellulose (MC), 37.5 μL of 8 M urea solution, 0.07 μL of acetic acid and 2.5 μL of ultrapure water. The loading mixture was then analyzed with iCE3 Capillary Isoelectric Focusing Analyzer equipped with a FC-COATED whole-column detection capillary. The focusing was carried out by two steps: (1) 1.5 kV for 1 minute; (2) 3 kV for 8 minutes, and the auto-sampler tray was maintained at 15° C. Absorbance detection took place at 280 nm. After the analysis, the raw data were processed with Empower 3.

EC50 FACS: Stable CHO-K1 cells expressing either human LILRB4 or flag-tagged cynomolgus monkey LILRB4 were used to determine binding capacity of stability samples of H7K3 antibody. CHO-K1 cells (1×10⁵) were stained with serially titrated stability samples followed by secondary staining with fluorescent-conjugated anti-human IgG (Biolegend). Geometric mean fluorescence (MFI) was measured and EC₅₀ were calculated by Prism.

EC50 ELISA: Human LILRB4 ECD-his recombinant protein (Sino Biological) or cynomolgus monkey LILRB4 ECD-his recombinant protein (ACRObiosystems) was coated onto EIA/RIA plates (Corning) overnight at 4° C. After blocking for 2 hours at 37° C. with 5% non-fat milk, 100 μL of serial diluted anti-LILRB4 antibodies were added into the well and incubated for 45 min at 37° C. Subsequently, the plates were washed with PBS-Tween 20 (0.05%) for three times, and PBS one time before incubated for 35 min with HRP-conjugated anti-hFc antibody (Jackson ImmunoResearch Laboratories) at room temperature. Signals were developed with TMB substrates (Sigma), stopped by the addition of 2 M sulfuric acid before read at 450 nm with a plate reader (Molecular Devices). EC₅₀ was calculated based on OD₄₅₀ measurement using Prism (GraphPad).

ApoE inhibition (IC50): A mouse T hybridoma cell line expressing LILRB4 extracellularly linked with the nuclear factor of activated T cells (NFAT)-GFP reporter system was used to screen ligand blocking capacity of H7K3 variants. When LILRB4-reporter cells (2×10⁴ per well) were co-cultured with immobilized recombinant APOE3 protein (Novoprotein Cat #CI02) at 10 μg/mL, GFP expression was induced by binding of APOE to receptor LILRB4 and the GFP signal was quantified using flow cytometry. In the presence of serially titrated LILRB4 blocking antibody H7K3, the GFP expression was reduced in a dose-dependent manner and IC₅₀ was calculated by flow cytometry signals.

Autologous ADCC: PBMCs were freshly isolated from healthy donors and cultured overnight in the presence of rhIL-2 at 50 ng/mL and serially titrated antibodies. Cells were stained with fluorescent conjugated anti-CD14, anti-CD19, anti-CD303, and anti-CD123 antibodies, and acquired by FACS Celesta to count live monocytes, pDC and B cells. Dead cells were excluded by adding 7-AAD. Percent of killing was calculated as 100−[(# of cells with antibody treatment)/(# of cells without antibody treatment)].

ADCC of THP-1-GFP cells: THP-1-GFP cells and freshly isolated PBMCs were co-cultured (E:T ratio=50:1) overnight in the presence of rhIL-2 at 50 ng/mL and serially titrated antibodies. Live THP-1-GFP cell count was obtained by gating on GFP+ and 7-AAD-cells. Percent of killing was calculated using the same formulation of autologous ADCC.

ADCC of MDSC: MDSC and NK cells from two different healthy donors were prepared. MDSC was generated by co-culturing PBMCs with SKMEL5 cells in the presence of GM-CSF at 40 ng/mL for 8 days and purified using CD33+ microbeads. IL-2 (100 ng/mL) was added for NK cell activation. MDSC:NK=1:2.5 (duplicates) with 50,000 of MDSC and 125,000 of NK cells were co-cultured for 21 hrs. After incubation of 21 hrs, cells were stained with CD14-FITC. THP-1-luc-GFP cells were used as positive control in the same setting. Percent of killing was calculated using the same formulation of autologous ADCC.

ADCP: Human monocytes were isolated from PBMCs from healthy donors using negative selection (Miltenyi Biotec) and cultured in X-vivo 10+10% FBS medium for 7 days in the presence of 50 ng/mL M-CSF (R&D system). In the last 24 hrs, 50 ng/mL of interferon gamma was added to prime macrophages. THP-1-GFP cells (2.5×10⁴) were co-cultured with macrophages (E:T=5:1) for 24 hrs in the presence of serially titrated anti-LILRB4 antibody, stained with RPE-conjugated anti-CD163 and anti-CD206. Live THP-1 cells were acquired by flow cytometry by gating on GFP+ cells. Percent of killing was calculated based on both absolute count of THP-1 cells and GFP+%.

T-cell mediated cytotoxicity: A FACS based approach was used to determine the ability of anti-LILRB4 to mediate tumor cell killing by naïve T cells. Human buffy coats were obtained from heathy donors and peripheral blood mononuclear cells (PBMC) were isolated from buffy coats by Ficoll Paque Plus (GE Healthcare Catalog No. 17-1440-03) density gradient cell separation. Pan T cells were further isolated from PBMCs using a human Pan T cell isolation kit (Miltenyi Biotec Catalog No. 130-096-535). 4×10⁵ freshly isolated human pan T cells were used as effector cells and 1×10⁵ THP-1-GFP were used as target cells in a 4:1 ratio. Human pan T cells, THP-1-GFP cells, and increasing concentrations of anti-LILRB4 antibody or isotype control human IgG1 (BioXcell Catalog No. BE0297) were mixed in 200 μL total in RPMI 1640 (Gibco Catalog No. 61870-036)+10% heat-inactivated FBS (Gibco Catalog No. 10082-147) in U-shaped 96-well plate and incubated for 48 hrs at 37° C. At the end of incubation, 40 μL of supernatant was collected for cytokine Luminex assay. 7-AAD (BD Pharmingen Catalog No. 559925) was added to cells and 100 μL of cells was acquired by FACS Celesta and the percentage of GFP-positive cells were measured. Flow cytometry data were analyzed using Flowjo software (Flowjo LLC) and cell cytotoxicity was calculated as: percent of cytotoxicity=100−([T/NT]×100), where T and NT are the percentages of GFP+ cells treated with or without test antibodies, respectively.

Cytokine assays by Luminex: Cell supernatants were tested using a custom 15 plex panel kit (R&D Systems). In order to perform the DA Bead assay using the wall less plate and the reagents of the 15 plex kit, the protocol of the R&D Systems kit was slightly modified. First, the DA Bead wall less plate was blocked for 30 minutes at room temperature with 10 μL 1% bovine serum albumin (BSA) in PBS. The DA Bead plate was subsequently washed once using the automatic washing station LT MX (Curiox Biosystems) with 0.1% BSA 0.05% Tween 20 in PBS (wash buffer). Each well received 7.5 μL of premixed magnetic beads. The appropriate wells then received 7.5 L of diluted samples, standards or blank. The DA Bead plate was then vortexed for approximately 10 seconds on an analog microplate Genie Shaker (Scientific Industries Inc., Bohemia, NY) at an intensity scale of 4. DA Bead plate was placed on a 3 mm span orbital shaker (Orbit 300, Labnet, Edison, NJ) and shaken for 120 minutes at 350 revolutions per minute (rpm) (0.2×g) at room temperature. DA Bead plate was then washed 3 times with the LT MX washing station. Each used well received 10 L of detection antibody diluent. The DA Bead was subsequently placed for approximately 10 seconds on an analog microplate genie shaker, as described above, and incubated on the orbital shaker for 60 minutes at 350 rpm at room temperature. The DA Bead plate was then washed 3 times using the LT MX station. Each well received 10 L of streptavidin phycoerythrin diluent. The DA Bead plate was placed for approximately 10 seconds on the Genie Shaker, as described above, and incubated for 30 minutes on the orbital shaker at 350 rpm at room temperature. The DA Bead plate was washed 3 times in the LT MX station. The beads were resuspended with a total volume of 65 L wash buffer and transferred to a skirted PCR plate and read in a Luminex reader for data acquisition (MAGPIX, a dual laser flow-based detection instrument, Luminex).

FACS analysis of co-cultured T-cells and THP-1 cells: Human buffy coats were obtained from heathy donors and peripheral blood mononuclear cells (PBMC) were isolated from buffy coats by Ficoll Paque Plus (GE Healthcare Catalog No. 17-1440-03) density gradient cell separation. Pan T cells were further isolated from frozen PBMCs by negative depletion using a human Pan T cell isolation kit (Miltenyi Biotec Catalog No. 130-096-535). 8×10⁵ purified human pan T cells were used as effector cells and 1×10⁵ THP-1-GFP were used as target cells in an 8:1 ratio. Human pan T cells, THP-1-GFP cells, and anti-LILRB4 antibody or isotype control human IgG1 (BioXcell Catalog No. BE0297) were mixed in 200 μL total in X-vivo 10 (Lonza Catalog No. 04-380Q)+10% heat-inactivated fetal bovine serum (FBS; Gibco Catalog No. 10082-147) in U-shaped 96-well plate and incubated for 48 hrs at 37° C. The final concentration of anti-LILRB4 antibody or human IgG1 was 3 μg/mL (20 nM). To measure intracellular TNFα and IFNγ production, protein transport inhibitor (BD Biosciences Catalog No. 555029) was added in the last 11 hrs of incubation. At the end of incubation, the cells were spun down out of the medium containing protein transport inhibitor and incubated with human IgG (Sigma Aldrich Catalog No. 14506) at room temperature for 10 minutes to block Fc receptors. The cells were then stained for surface antigens with directly conjugated anti-CD4 (Catalog No. 564975) and anti-CD8 (Catalog No. 563256), fixed and permeabilized using Fixation/Permeabilization kit (Catalog NO. 555028), and stained with anti-IFNγ (Catalog No. 554552) and anti-TNFα (Catalog No. 554514) or isotype control antibodies (Catalog NO. 554681, 555749). The antibodies against surface and intracellular antigens are from Becton, Dickinson and Company.

FACS analysis of T-cell and THP-1 cell activation: Surface expression of T-cell activation markers was evaluated with directly conjugated anti-CD4 (Catalog No. 564975), anti-CD8 (Catalog No. 563256), anti-CD69 (Catalog No. 555533), and anti-CD25 (Catalog No. 555432) antibodies from BD Biosciences. Surface expression of THP-1 cell markers was assessed with directly conjugated anti-HLA-DR (Catalog No. 559866), anti-CD80 (Catalog No. 563084), anti-CD83 (Catalog No. 565336), anti-CD86 (Catalog No. 562432), anti-CD205 (Catalog No. 558156), anti-CD87 (Catalog No. 743096) from BD Biosciences, and anti-CD40 (Catalog No. 334310), anti-HLA-A, B, C (Catalog No. 311406), anti-LILRB4 (Catalog No. 333008) from Biolegend. Cells were acquired by FACS Celesta after 7-aminoactinomycin D (7AAD; BD Pharmingen Catalog No. 559925) was added to cells. Flow cytometry data were analyzed using Flowjo software (Flowjo LLC) and graphed by Prism GraphPad software.

PCR fragments for CAR-encoding fragments: PCR were performed with PRIMESTAR DNA Polymerase (Takara Bio R010B) using Venti thermocycler using the follow conditions: 98° C. 30 s, (98° C._10 s, 64° C._5 s 72° C._30 s)×35 cycles, 72° C._7 min. The PCR products were further purified using PCR clean up kit (Macherey-Nagel 740609.250) and the eluted DNA were further washed/concentrated by ethanol precipitation.

Gene targeting: The inventors used TransAct to activate T cells first, then wash off TransAct before electroporation. 72 h after TransAct activation of PMBC, the CD3/CD28 beads were magnetically removed, and the T cells were transfected by 5 ug and 100 pmol/l RNA duplex using neon transfection system (thermo fisher, 10 ul tip). 4×10⁵ cells were mixed with the RNP complex and 2.5 ug PCR fragment encoding CAR. Following electroporation cells were diluted into culture medium and incubated at 37° C., 7% CO2. Subsequently, edited cells were cultured using standard conditions (37° C. and expanded in T-cell growth medium, replenished as needed to maintain a density of ˜1×10⁶ cells per ml every 2 to 3 days).

TCRalpha KO T cells: Human primary T cells were transfected with CRISPR-Cas9 RNP complexes including a guide RNA targeting the 5′ end of the first exon of TRAC and supplied with DNA template for homologous recombination-based knock in. After knocking out TCRalpha, the cells were grown in complete Optimizer medium with IL-2 300 IU/ml and without anti-CD3/28 added. Following transfection, cells were expanded in culture for 2 weeks.

Flow cytometry assays of CAR-T transduction: LILRB4 CAR-T were identified by binding to LILRB4-Fc fusion protein (ACRObiosystems CDK-H5259) and anti-Fc antibody (Biolegend B278652). Success in knock-out of the endogenous TCR alpha (TRAC) locus (KO) was measured by anti-CD3 staining (anti-CD3 PE, BD 555333).

Antigen stimulation assays: 1 μg/ml recombinant control antigen or LILRB4 antigen were coated on 96 well plate overnight in PBS buffer. Plate were washed twice with PBS buffer. 1×10⁵ CAR-T cells in culture media (without any cytokine added) were added to each well and incubated for 72 hours. Cell culture supernatant were collected for cytokine release measurement by Luminex assay (R&D Systems FCS™-18).

CAR-T cell mediated cytotoxicity assays: CHO K1 RB4 cells were seeded at different density (6×10⁴, 2×10⁴ or 7×10³) for 12 hours. 1×10⁵ CAR-T cells were added and cytotoxicity were measured by removing the supernatant CAR-T cells and washing the plate 2 times with PBS. Total viable adherent CHO K1 RB4 cells were measured by Promega CTG2.0 luminescence kit, and the % cytotoxicity was calculated by dividing the Luminescent signal of each condition with the same E:T ratio activated T cell control.

Phase 1 design: During the Part 1A monotherapy escalation phase (single dose on Day 1) patients will be enrolled into sequential cohorts of increasing doses of anti-LILRB4 monotherapy. The goal of Part 1A is to determine the maximum tolerated dose (MTD) of anti-LILRB4 monotherapy (MTD1). DLTs for MTD1 will be evaluated during the first 14 days of treatment (prior to the first dose of anti-LILRB4 in combination with azacytidine/azacitidine), i.e., the first dose-interval for anti-LILRB4. The initial dose-escalation begins with an accelerated titration design followed by a standard escalation phase that will use a 3+3 design. Part 1 will include both relapsed and/or refractory myelomonocytic (M4) and monocytic/monoblastic (M5) AML patients and chronic myelomonocytic leukemia (CMML) patients as described above. This 2-week monotherapy lead-in (“window”) of anti-LILRB4 enables the studying of the effects of a monoclonal antibody that specifically targets LILRB4 as monotherapy. During Part 1B (starting on Day 15), patients without DLTs during Part 1A will receive the same dose of anti-LILRB4 that was administered in Part 1A in combination with a standard dose of azacytidine/azacitidine (75 mg/m² subcutaneously for 7 days every 28 days). The MTD of anti-LILRB4 in combination with azacytidine/azacitidine (MTD2) will be determined in the 28-day DLT window, consisting of the 14 days of monotherapy and 14 days of the combination treatment. The overall DLT period of Part 1 (Part 1A and Part 1B combined) is 28 days. Subsequent cycles will be anti-LILRB4 in combination with azacytidine/azacitidine.

### Example 2

The inventors previously identified a rabbit anti-LILRB4 antibody named B4-193 that has a high binding affinity to human LILRB4 and can inhibit cancer development in a xenograft AML mouse model. (see U.S. Provisional Application No. 62/730,715, the disclosure of which is incorporated herein in its entirety)

The inventors generated a humanized anti-LILRB4 antibody that contains the same CDRs as B4-193. The humanized anti-LILRB4 antibody, named H7K3, has a heavy chain variable region sequence of SEQ ID NO: 1 and a light chain variable region sequence of SEQ ID NO: 3.

The in silico analysis of the H7K3 indicated that it has a potential oxidation site at the amino acid residue W in the heavy chain CDR3 and a potential deamidation site at the amino acid residues NS in the light chain CDR1, which may potentially decrease the stability of the antibody.

The inventors then assessed the stability of the antibody H7K3 at 40° C. in PBS or a formulation buffer, the results of which are summarized in the Tables 2 and 3. The results indicate that the antibody H7K3 is unstable.

TABLE 2
Stability of H7K3 in PBS

| | SEC | icIEF | FACS | FACS |
|---|---|---|---|---|
| | (HMW/MP/LMW) | (Acidic/MP/Basic) | Human EC50 nM | Cyno EC50 nM |
| H7K3 | | | 0.6501 | 1.185 |
| T₀ | 3.7/96.3/0 | 20.9/69.5/9.6 | 0.7337 | 1.205 |
| 2 w 40° C. | 4.3/93.8/1.9 | 88.8/9.4/1.8 | 0.8954 | 1.706 |
| 4 w 40° C. | 3.6/93.2/3.2 | 94.9/4.3/0.7 | 0.6956 | 1.72 |
| Changes at | Very slight change | Dramatic increase | No change in | Decrease in |
| 40° C. | | in acidic species | binding | binding |

TABLE 3
Stability of H7K3 in Formulation buffer

| | SEC | icIEF | FACS | FACS |
|---|---|---|---|---|
| | (HMW/MP/LMW) | (Acidic/MP/Basic) | Human EC50 nM | Cyno EC50 nM |
| H7K3 | | | 0.6501 | 1.185 |
| T₀ | 1.0/99.0/0 | 17.1/72.3/10.6 | 0.5574 | 0.8835 |
| 2 w 40° C. | 1.0/98.6/0.4 | 52.8/39.2/8.0 | 1.424 | 3.806 |
| 4 w 40° C. | 1.2/98.0/0.4 | 71.7/23.1/5.2 | 2.144 | 6.051 |
| Changes at | Very slight change | Dramatic increase | Decrease in | Log decrease in |
| 40° C. | | in acidic species | binding | binding |

### Example 3

To reengineer H7K3 to correct deamination and oxidation liabilities, the inventors generated a series of variants having mutations at the potential oxidation site and deamination site. The sequences of the H7K3 variants are summarized in the Table 4.

TABLE 4
H7K3 variants

| | | Heavy Chain | Light chain |
|---|---|---|---|
| Variant | Mutation | variable sequence | variable sequence |
| 1 | H7m1 (W/V) | SEQ ID NO: 11 | SEQ ID NO: 3 |
| 2 | H7m2 (W/Y) | SEQ ID NO: 13 | SEQ ID NO: 3 |
| 3 | H7m3 (W/F) | SEQ ID NO: 15 | SEQ ID NO: 3 |
| 4 | H7m4 (W/Q) | SEQ ID NO: 17 | SEQ ID NO: 3 |
| 5 | K3m1 (N/V) | SEQ ID NO: 1 | SEQ ID NO: 19 |
| 6 | K3m2 (N/D) | SEQ ID NO: 1 | SEQ ID NO: 21 |
| 7 | K3m3 (N/E) | SEQ ID NO: 1 | SEQ ID NO: 23 |
| 8 | K3m4 (N/Q) | SEQ ID NO: 1 | SEQ ID NO: 25 |
| 9 | K3m5 (N/S) | SEQ ID NO: 1 | SEQ ID NO: 27 |
| 10 | K3m6 (N/T) | SEQ ID NO: 1 | SEQ ID NO: 29 |
| 11 | K3m7 (S/Q) | SEQ ID NO: 1 | SEQ ID NO: 31 |
| 12 | K3m8 (S/V) | SEQ ID NO: 1 | SEQ ID NO: 33 |

The inventors tested the binding affinity of the H7K3 variants via FACS using CHO-stable cells expressing human and cyno LILRB4. The results are summarized in the Table 5 below.

TABLE 5
Binding affinity of H7K3 variants

| | | EC50 (nM) | EC50 (nM) |
|---|---|---|---|
| Variant | Mutation | Human | Cyno |
| 5 | K3m1 (N/V) | 0.4626 | 1.233 |
| 8 | K3m4 (N/Q) | 0.4796 | 0.9889 |
| 9 | K3m5 (N/S) | 0.4152 | 1.125 |
| 11 | K3m7 (S/Q) | 0.4552 | 0.9741 |
| 12 | K3m8 (S/V) | 0.402 | 0.9331 |
| WT | K3 | 0.5431 | 1.333 |

The inventors further tested the stability of the H7K3 variants. Oxidized species was observed (confirmed by mapping results) in H7K3 after incubation at 40° C. for 4 W compared to the control sample without incubation at 40° C., as summarized in the Table 6 below. No obvious oxidation was observed in H7K3m5 after incubation at 40° C. for 4 weeks. It can be concluded that H7K3m5 is more stable than H7K3.

TABLE 6
Comparison in Deglycosylated Reduced Mass (DRM) results of H7K3 and H7K3m5

| | | | | Theoretical | Measured | Difference |
|---|---|---|---|---|---|---|
| Molecule | Buffer | Condition | Subunit Modification | Mass (Da) | Mass (Da) | (ppm) |
| H7K3 | 20 mM | Control | LC: partial reduced | 23601.3 | 23601.0 | −12.7 |
| | Histidine-HCl, | | HC: partial reduced, | 48969.3 | 48969.9 | 12.3 |
| | pH 6.0, | | deglycosylated, —K | | | |
| | 7.0% sucrose, | Incubation | LC: partial reduced | 23601.3 | 23601.7 | 16.9 |
| | 0.02% PS80 | at 40° C. | LC: partial reduced, | NA | 23583.9 | NA |
| | | for 4 W | with modification | | | |
| | | | LC: partial reduced, | 23617.3 | 23617.7 | 16.9 |
| | | | Oxide | | | |
| | | | LC: partial reduced, | 23633.3 | 23633.8 | 21.2 |
| | | | 2*Oxide | | | |
| | | | HC: partial reduced, | NA | 48992.4 | NA |
| | | | deglycosylated, —K, | | | |
| | | | 2*Oxide, with unknown | | | |
| | | | modification | | | |
| H7K3m5 | | Control | LC: partial reduced | 23574.3 | 23574.1 | −8.5 |
| | | | HC: partial reduced, | 48969.3 | 48969.6 | 6.1 |
| | | | deglycosylated, —K | | | |
| | | Incubation | LC: partial reduced | 23574.3 | 23574.0 | −12.7 |
| | | at 40° C. | HC: partial reduced, | 48969.3 | 48969.6 | 6.1 |
| | | for 4 W | deglycosylated, —K | | | |

The inventors further tested the stability of the H7K3 variants. About ˜50% decrease in main peak were observed in H7K3 after incubation at 40° C. for 4 weeks compared to the control sample without incubation at 40° C. About 16% decrease in main peak 00 were observed in H7K3m5 after incubation at 40° C. for 4 weeks compared to the control. It can be concluded that H7K3m5 is more stable than H7K3.

TABLE 7
Comparison in icIEF results of H7K3 and H7K3m5

| | | | pI of | Area % | | |
|---|---|---|---|---|---|---|
| | | Buffer | main | Acidic | Main | Basic |
| Molecule | Sample | Composition | peak | peaks | peak | peaks |
| H7K3 | Control | 20 mM | 8.8 | 17.1 | 72.3 | 10.6 |
| | 40° C. | Histidine-HCl, | 8.8 | 52.8 | 39.2 | 8.0 |
| | for 2 W | 7% sucrose, | | | | |
| | 40° C. | 0.02% PS80, | 8.8 | 71.7 | 23.1 | 5.2 |
| | for 4 W | pH 6.0 | | | | |
| H7K3m5 | Control | | 8.8 | 26.3 | 62.5 | 11.1 |
| | 40° C. | | 8.8 | 34.3 | 52.4 | 13.3 |
| | for 2 W | | | | | |
| | 40° C. | | 8.8 | 41.6 | 46.4 | 11.9 |
| | for 4 W | | | | | |

No significant oxidation was detected in H7K3m5 under the light exposure. Consistency between DRM and peptide mapping results was obtained for H7K3m5 with light exposure. The results indicated that H7K3m5 showed acceptable stability under oxidizing environment (light).

For H7K3m5 after incubation at 40° C. for 4 weeks, the DRM results indicated there is no significant oxidation. H7K3m5 appears more stable than H7K3 regarding DRM and icIEF results. icIEF is capable of monitoring the oxidation and deamidation even with low ratio.

### Example 4

This example illustrates the biological function of H7K3m5.

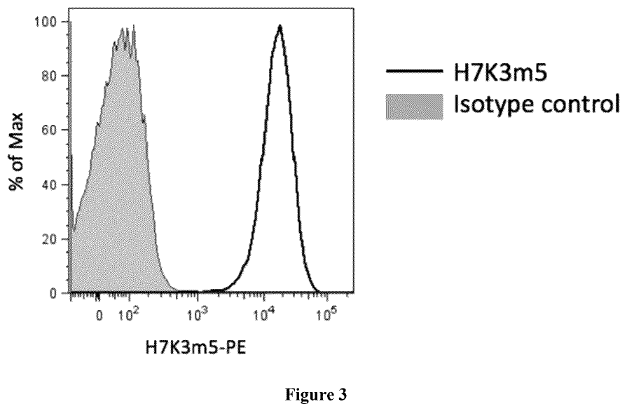

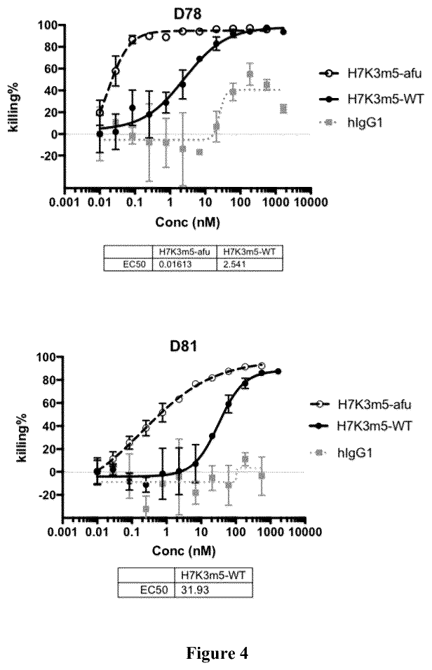

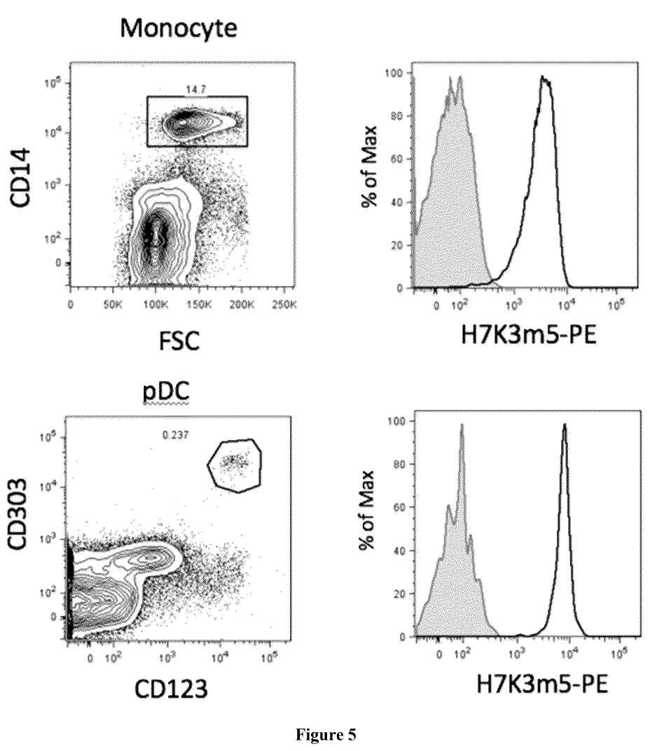

The inventors first assessed whether H7K3m5 binds to the LILRB4 expressed on cell surface. As shown in FIG. 3, H7K3m5 recognized LILRB4 expressed on THP-1-GFP cells. H7K3m5 is capable of inducing ADCC activity against THP-1 cells in the presence of PBMC in vitro, as shown in FIG. 4. Notably, afucosylated H7K3m5 showed enhanced cell killing compared to wild-type H7K3m5, with EC50 enhanced by more than 100-fold. As shown in FIG. 5, H7K3m5 also binds to LILRB4 expressed on human monocytes and plasmacytoid dendritic cells (pDC).

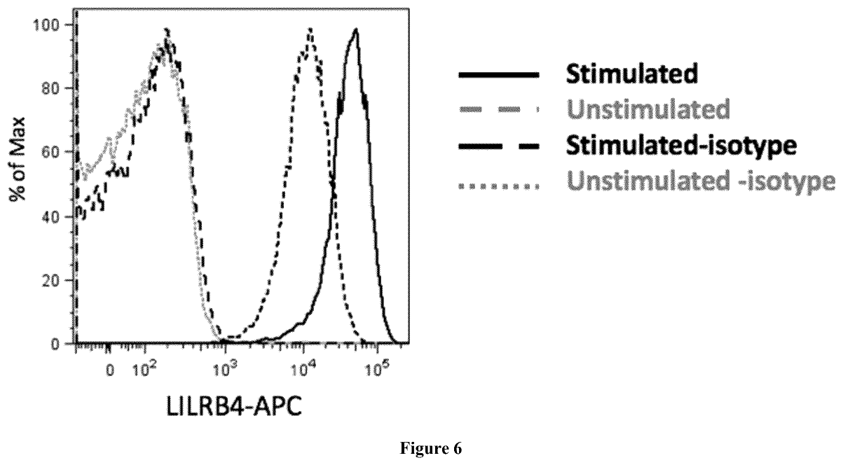

As shown in FIG. 6, LILRB4 expression in human monocytes can be up-regulated by IL-10 and IFNα treatment. Human monocytes were isolated from human PBMC by negative selection, and stimulated by 50 ng/ml IL-10 plus 1500 U IFNα in RPMI medium with 10% FBS and 1×L-glutamin for 24 hours. Cells were stained by CD14-PE and anti-LILRB4-APC at 2 μg/ml.

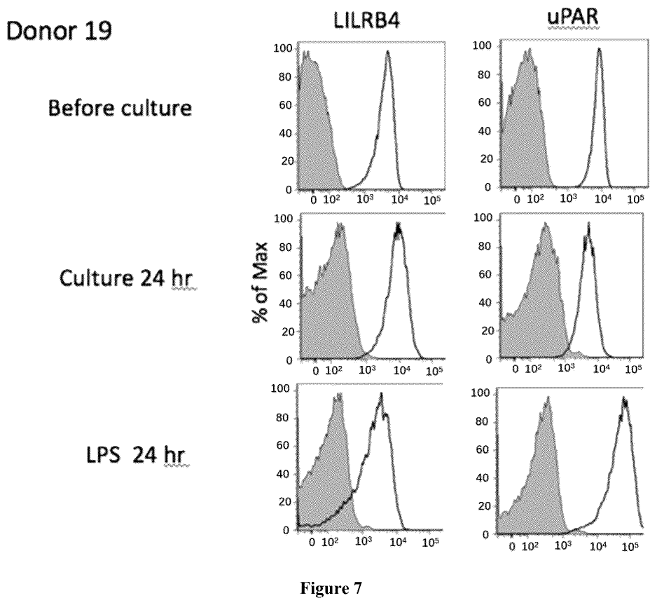

On the other hand, human monocytes treated by LPS down-regulated LILRB4 expression and increased uPAR expression level (FIG. 7).

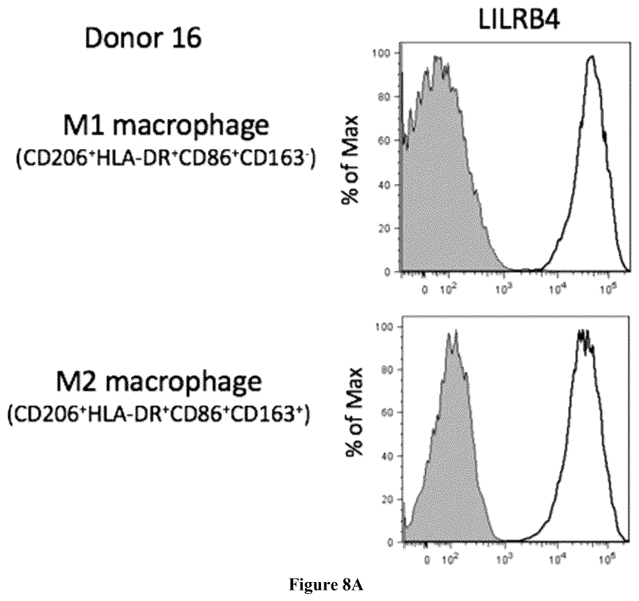

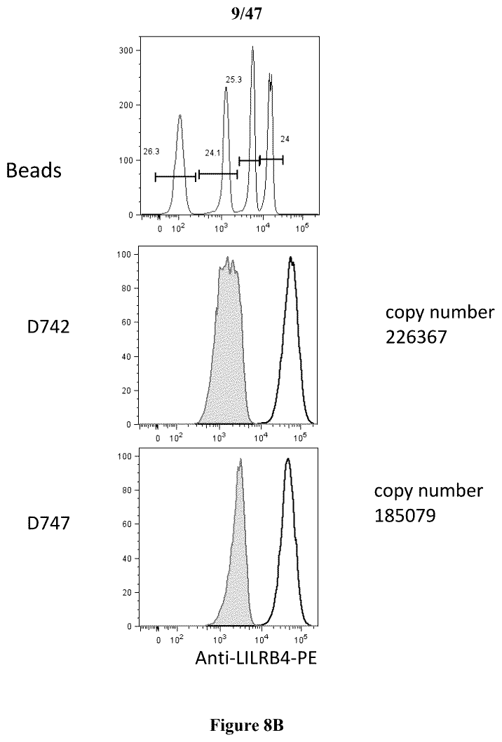

As shown in FIGS. 8A and 8B, LILRB4 is expressed on in vitro differentiated human macrophages. The copy number of LILRB4 on in vitro monocyte-derived human macrophages is very high, close to 150,000 copies/cell.

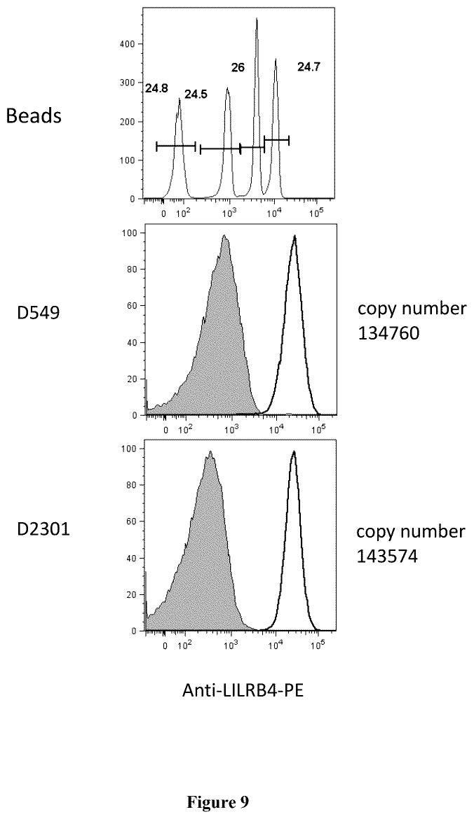

As shown in FIG. 9, LILRB4 expression is greatly increased in myeloid-derived suppressor cells (MDSC). The copy number of LILRB4 on in vitro differentiated MDSCs can be as high as 200,000 copies/cell.

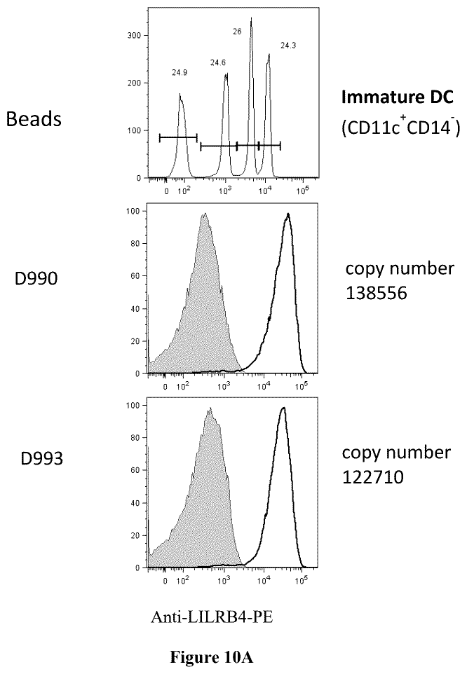

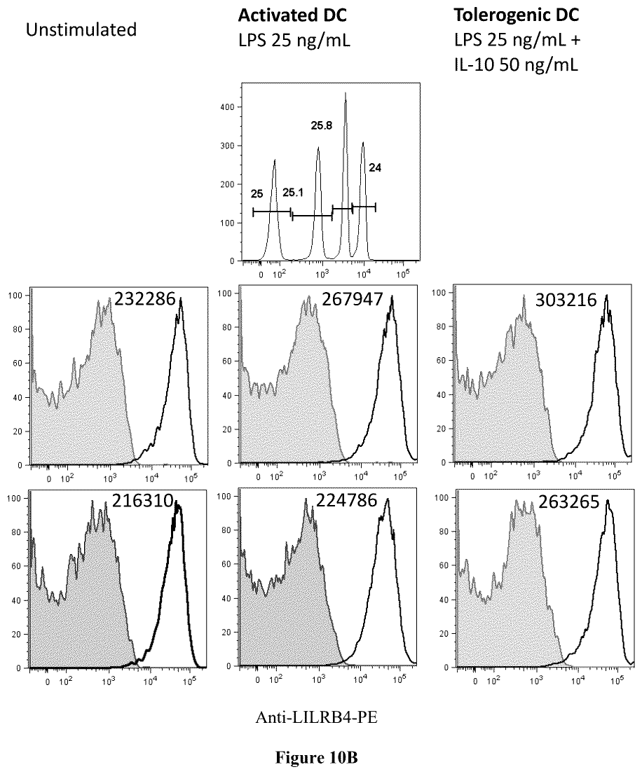

As shown in FIGS. 10A-10B, LILRB4 is also highly expressed on human monocyte-derived dendritic cells (DCs). LILRB4 level is seen in the following order from high to low: Tolerogenic DC>Activated DC>Immature DC.

TABLE 8
Comparison of copy numbers of LILRB4 on different
types of human primary cells and AML cell line

| | Range of LILRB4 | Average of LILRB4 |
|---|---|---|
| Cell type | copy number/cell | copy number/cell |
| Human monocyte from | 2355-7886 | 5061 |
| healthy donors (n = 12) | | |
| In vitro differentiated | 134,760; 143,574 | 139,167 |
| MDSCs (n = 2) | | |
| In vitro monocyte-derived | 226,367; 185,079 | 205,723 |
| macrophages (n = 2) | | |
| AML cell line THP-1 | 57,211 | 57,211 |
| (n = 1) | | |

The inventors then compared the LILRB4 mRNA expression levels between solid tumor samples with high and low signal for macrophage infiltration. RNA sequencing data from the TCGA database was analyzed using computational biology and statistical approaches to identify samples presenting coordinated up-regulated expression of multiple transcripts primarily expressed by tumor-associated macrophages (collectively defining a macrophage gene expression “signature”) within each tumor type. Samples presenting high signal for the macrophage gene expression “signature” were classified as highly infiltrated by tumor-associated macrophages. The expression levels of LILRB4 transcript were then compared between those tumor samples and the remaining samples in which a macrophage gene expression “signature” was not apparent (grouped as low macrophage infiltrated samples). As shown in FIG. 11, higher LILRB4 mRNA expression levels are correlated to the macrophage infiltration.

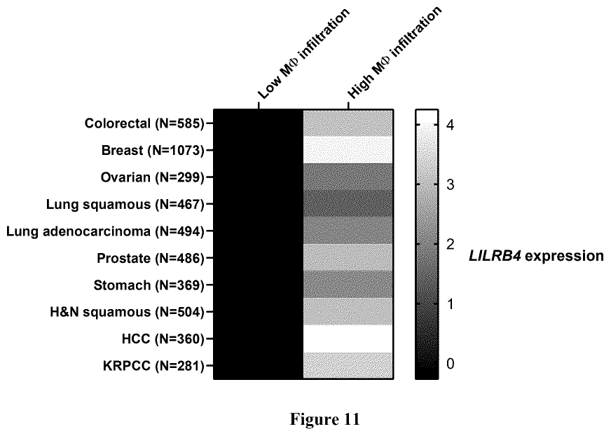

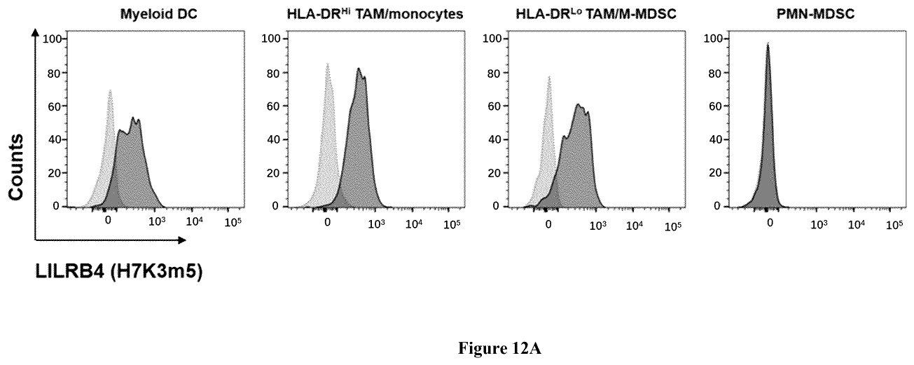

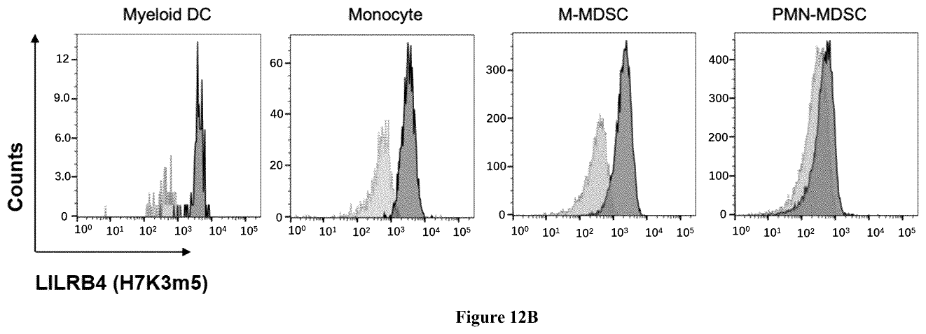

The inventors then determined the type of cells bound by LILRB4 antibody (H7K3m5) in solid tumors. Tissue samples from solid tumors were dissociated into single cells using mechanical methods and PBS-10 mM EDTA. In some cases, a peripheral blood sample was also obtained from same donor as tumor tissue sample and processed for flow cytometric analysis using standard methods. The resultant cells were incubated with H7K3m5 and an antibody cocktail for markers of myeloid cells at 4° C., using standard methods, and the stained samples were analyzed by flow cytometry. The gating in tumor samples were as follows. Myeloid dendritic cells (DC, CD11b⁺CD15⁻CD14⁻HLA-DR⁺CD11c⁺), HLA-DR^{Hi} tumor-associated macrophages (TAM)/monocytes (CD11b⁺CD15⁻CD14⁺HLA-DR⁺), HLA-DR^{Lo} TAM/monocytic myeloid-derived suppressor cells (M-MDSC, CD11b⁺CD15⁻CD14⁺HLA-DR⁻), PMN-MDSC (CD11b⁺CD15⁺CD14⁻). The gating in the peripheral blood were as follows: myeloid DC (CD11b⁺CD14⁻CD11c⁺), monocytes (CD11b⁺CD14⁺HLA-DR^{Hi}), M-MDSC (CD11b⁺CD14⁺HLA-DR^{Lo}), PMN-MDSC (CD11b⁺CD15⁺CD14⁻HLA-DR⁻Lox-1⁺). As shown in FIGS. 12A-12B, the results of these experiments demonstrate that H7K3m5 binds monocytic (but not granulocytic) myeloid cells in the tumor microenvironment and in the periphery. This finding is consistent with the expression pattern of LILRB4, which is restricted to myeloid cells of the monocytic lineage.

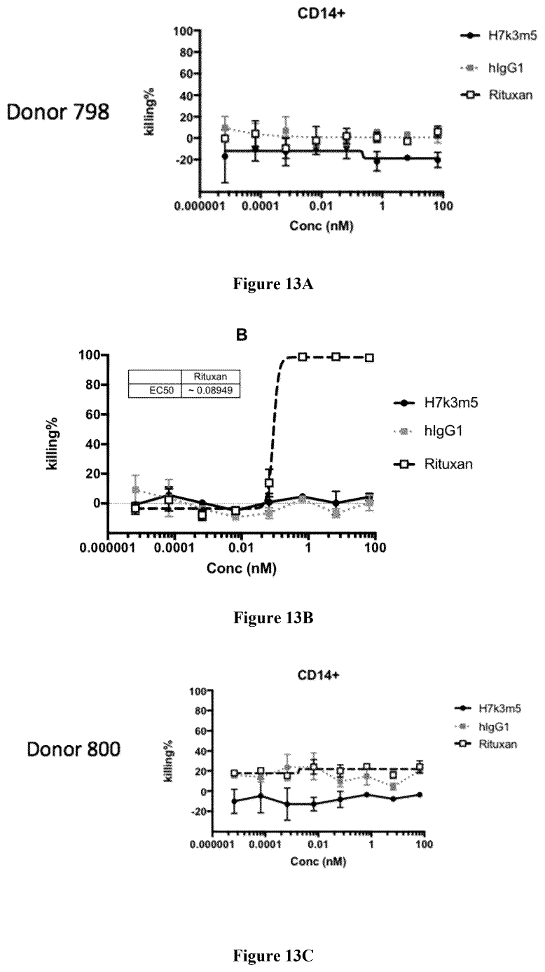

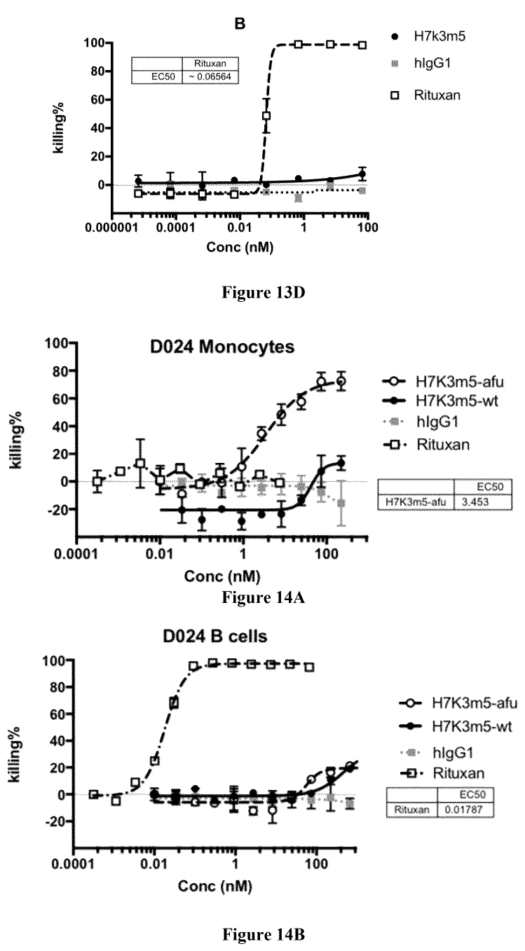

Although LILRB4 is expressed on primary normal monocytes, binding of H7K3m5 did not result in monocyte killing via ADCC (FIGS. 13A-13D). With afucosylated H7K3m5, killing of normal monocytes via ADCC was observed in 25-50% of tested PBMC donors (FIGS. 14A-14D, and FIGS. 15C-15D). In addition, as shown in FIGS. 15A-15B, both afucosylated and wild type H7K3m5 resulted in killing of pDCs via autologous ADCC. In the meantime, monocytes may be killed only with afucosylated H7K3m5, depending on donors (FIGS. 15C-15D).

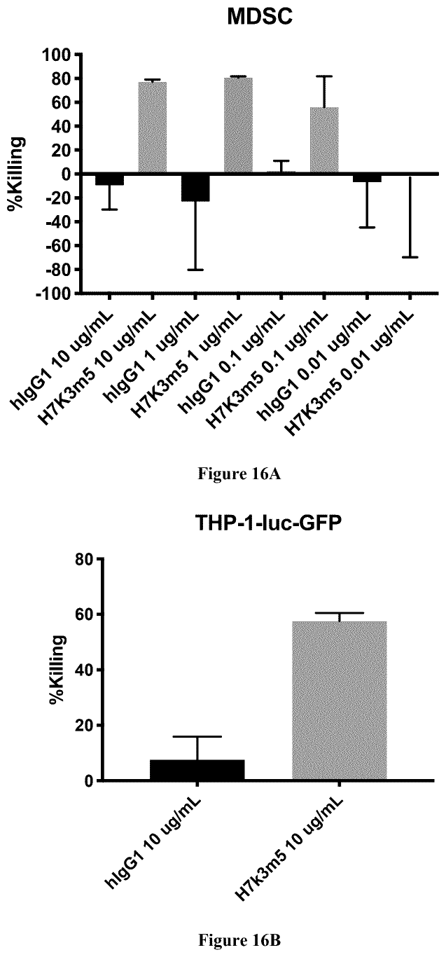

As shown in FIGS. 16A and 16B, anti-LILRB4 antibody such as H7K3m5 depleted in vitro-derived (tumor cell conditioned) myeloid-derived suppressor cells (MDSCs) via ADCC. This is a potential mechanism of action for anti-LILRB4 in the treatment of solid tumors.

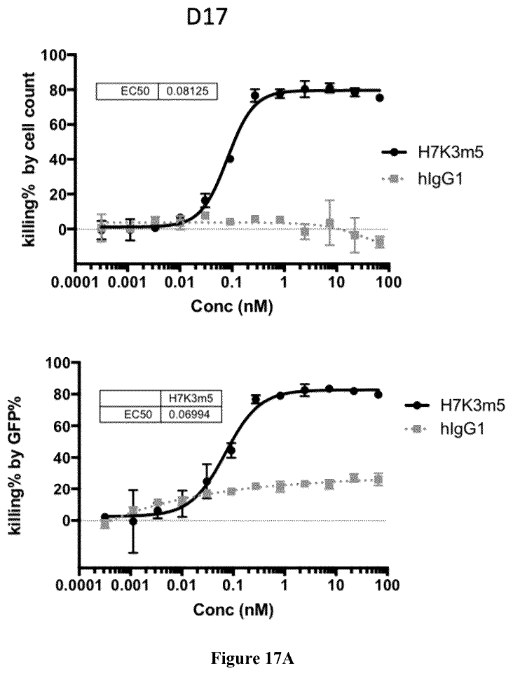

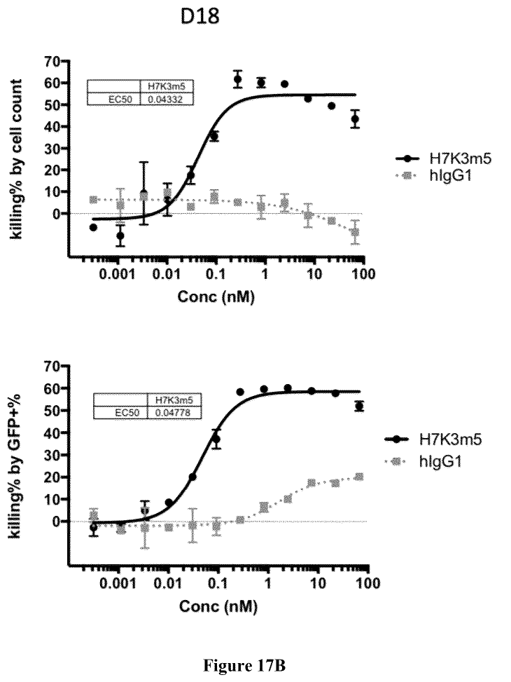

Besides ADCC, wild type H7K3m5 also demonstrated cell killing via ADCP towards THP-1 cells (FIGS. 17A-17B).

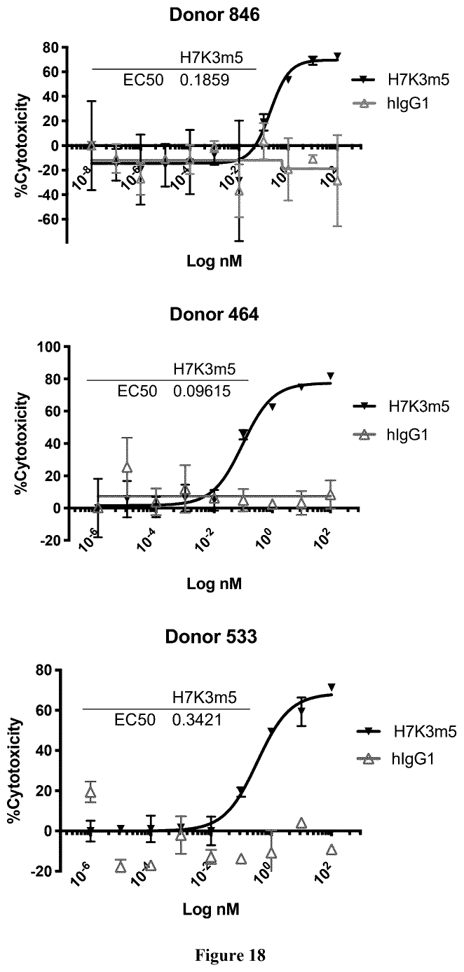

Anti-LILRB4 antibody also enhances T-cell mediated cytotoxicity against tumor cells. As shown in FIG. 18, anti-LILRB4 can induce T-cell cytotoxicity against AML cell line THP-1-GFP whereas no T cell cytotoxicity was observed with isotype control antibody. H7K3m5 treatment induced naïve T cells to kill THP-1 AML cells in a dose-dependent manner. The mean EC50 was 0.208±0.125 nM (31.2±18.8 ng/mL) (n=3).

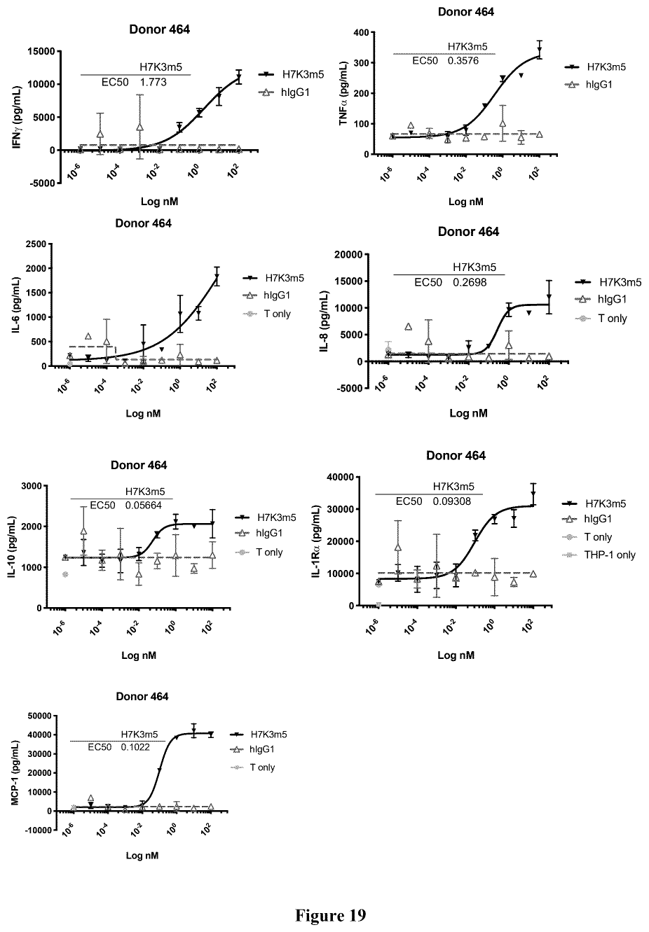

Cytokine measurement by a multiplexing Luminex assay from the supernatant of cytotoxicity assay samples demonstrated a dose-dependent increase of IFNγ and TNFα in H7K3m5 treated samples compared to control samples (FIG. 19). The cytokine profile changes were consistent with increased cell killing activity measured by THP-1 cell quantification (FIG. 18). In addition to increased TNFα and IFNγ levels which are likely produced by activated cytotoxic T cells, a dose-dependent increase of MCP-1 and IL-1Rα known produced by monocytes and macrophages were also observed, which are likely from activated THP-1 cells. IL-6, IL-8, IL-10 levels were also increased in respond to H7K3m5 treatment in comparison to controls. These cytokines are likely from both activated T cells and THP-1 cells.

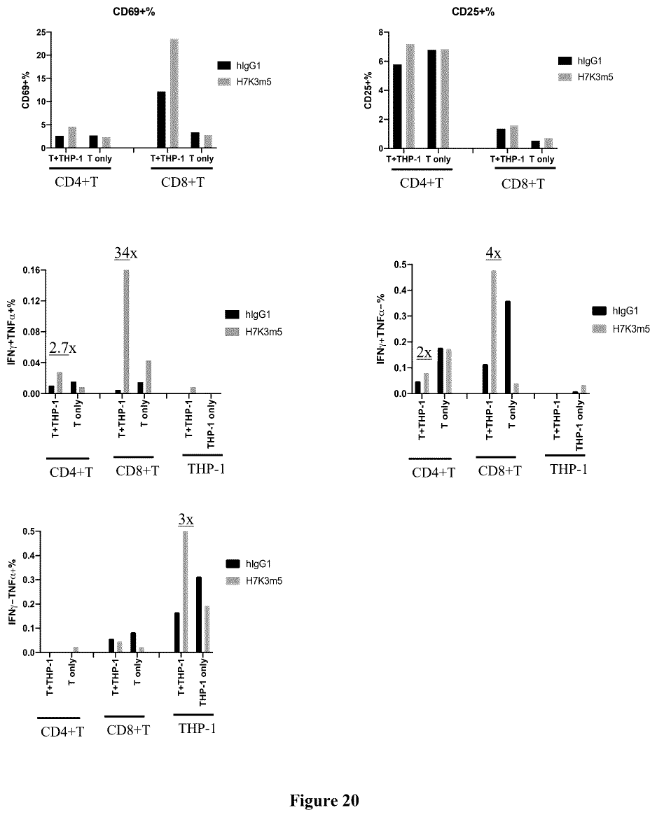

T-cell activation markers were assessed by flow cytometry (see FIG. 20). A 2-fold higher percentage of CD69-expressing T cells was observed with H7K3m5 treatment (FIG. 20A). Here, 4.7% of CD4+ T cells and 23.6% of CD8+ T cells treated with H7K3m5 expressed CD69 while only 2.6% of CD4+ T cells and 12.2% of CD8+ T cells treated with isotype control expressed CD69. There was no increase of CD69+ T cells by H7K3m5 treatment when T cells were cultured alone. H7K3m5 had only mild effect on CD25+ T-cell increase (FIG. 20B).

Cytokine production in the co-cultured T cells and THP-1 AML cells was also assessed by intracellular staining for flow cytometry. Compared to isotype control, H7K3m5 treatment increased IFNγ and TNFα producing CD4+ T cells and CD8+ T cells by 2.7-fold and 34-fold, respectively (FIG. 20C). IFNγ-producing CD4+ and CD8+ T cells were increased by 2-fold and 4-fold with H7K3m5 treatment, and IFNγ was not produced by THP-1 cells (FIG. 20D). On the contrary, TNFα-producing THP-1 cells was increased by 3-fold with H7K3m5 treatment when co-cultured with T cells (FIG. 18E). Without T cells, H7K3m5 treatment on THP-1 cells alone did not result in an increase of TNFα-producing THP-1 cells. These data indicated that IFNγ was only produced by activated T cells and TNFα was produced by both activated T cells and THP-1 AML cells.

Taking together, H7K3m5 enhanced antigen presentation of THP-1 cells and activated T cells to produce IFNγ and TNFα.

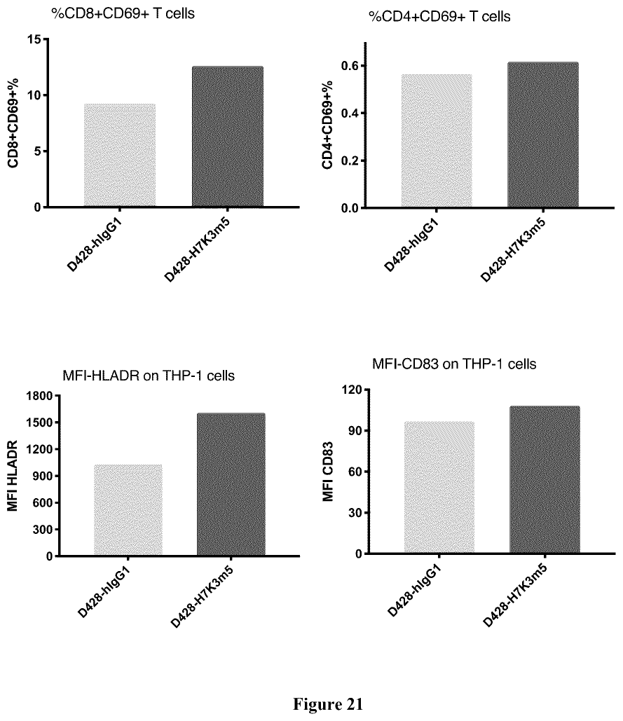

The proposed mechanism of enhancement of T-cell mediated cytotoxicity consists of blockade of LILRB4 inhibitory receptor signaling with the anti-LILRB4 blocking antibody, reduction of arginase production by THP-1 cells, production of cytokines by THP-1 cells (FIGS. 19 and 20) and enhanced antigen presentation ability of THP-1 (FIG. 21). As shown in FIG. 21, THP-1 AML cells and naïve T cells were co-cultured at 37° C. for 48 hrs at E:T ratio of 4:1 in the presence of H7K3m5 or human IgG1 at 1.5 ug/mL (10 nM). T-cell and THP-1 cell activation markers were assessed by flow cytometry (FIG. 21). On THP-1 AML cells, a mild up-regulation of costimulatory molecule CD83 was detected in all experiments. Each experiment used pan T cells from a different donor (n=4). A mild up-regulation of MHC class II molecule HLADR (n=3), MHC class I molecule (n=1), and another costimulatory molecule CD86 (n=2) was also detected on THP-1 cells. These data indicate that H7K3m5 treatment enhances antigen-presentation activity of THP-1. T-cell activation marker CD69 was induced by H7K3m5 treatment moderately (n=1), consistent with increased cytotoxic activity. Taking together, the enhanced antigen presentation of THP-1 may trigger T-cell activation and lead to enhanced cytotoxic activity.

When THP-1 cells are co-cultured with naïve T cells in the presence of H7K3m5, H7K3m5 activates T-cell cytotoxicity against THP-1 (FIG. 20). To further understand the changes to both cell types in this activity, naïve T cells and THP-1 AML cells were co-cultured at 37° C. for 48 hrs at an E:T ratio of 8:1 in the presence of H7K3m5 or human IgG1 at 3 μg/mL (20 nM). THP-1 activation was evaluated by flow cytometry (FIG. 22). Expression of HLA class I (A, B, C) and Class II (HLA-DR), co-stimulatory molecule CD40, CD86, CD80, and CD83 was induced by H7K3m5 treatment, indicating that H7K3m5 enhanced antigen-presentation function of THP-1 cells. On the contrary, expression of inhibitory molecules CD205 and LILRB4 was not reduced by H7K3m5. Interestingly and unexpected, H7K3m5 treatment also increased expression of uPAR, a NF-kB target downstream of LILRB4, which is highly expressed in monocytic AML cells and well known to promote cancer invasion, metastasis, survival and angiogenesis (Deng et al 2018).

When coculturing THP-1 AML cells with naïve T cells, no T-cell activation and cytotoxicity was observed. This is likely due to compromised antigen presentation function of THP-1 AML cells that have high LILRB4 expression. When H7K3m5 was added to co-cultured THP-1 and naïve T cells, enhanced antigen presentation function of THP-1 cells was observed exemplified by increased HLA-DR and CD83 expression. Activation of naïve T cells were correspondingly observed, in particular cytotoxic activation against THP-1 cells. This is corroborated by the increased secretion of cytokines (i.e., TNFα and IFNγ) in tissue culture media. These data demonstrate that H7K3m5 is able to activate T cell cytotoxicity against THP-1 AML cells.

Cytotoxicity of normal monocytes by naïve T cells mediated by H7K3m5 was also evaluated in this in vitro system but no killing of normal monocytes was observed (data not shown). LILRB4 density on normal monocytes is 10-fold lower than that on THP-1 AML cells, more importantly, the antigen presentation capacity of normal monocytes is expected to be significantly lower than that of tumor AML cells due to lack of tumor associated antigens. Although the inventors found that patient AML cells may express LILRB4 at the similar or high levels as that on normal monocytes, it is plausible that AML blasts may be phenotypically more like THP-1 cells and can be killed by activated T cells when treated with H7K3m5.

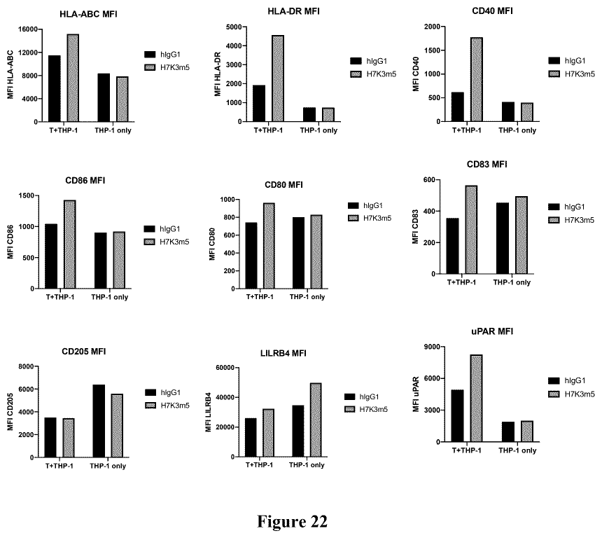

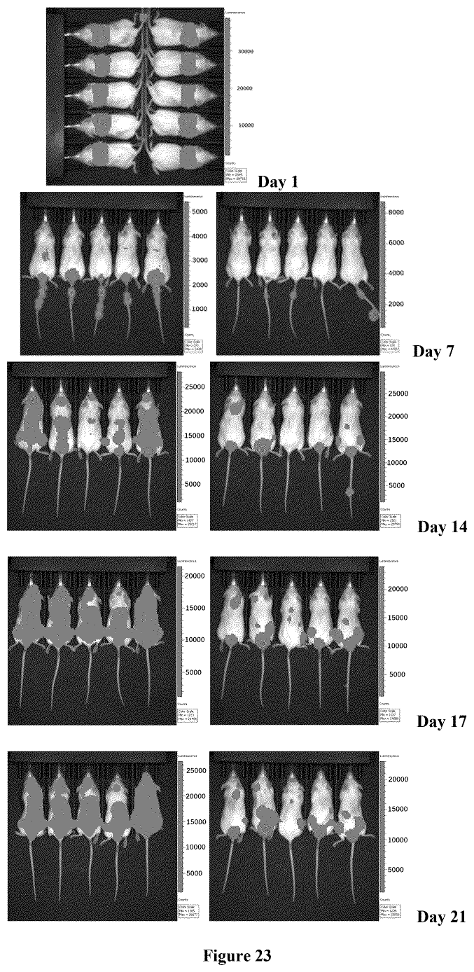

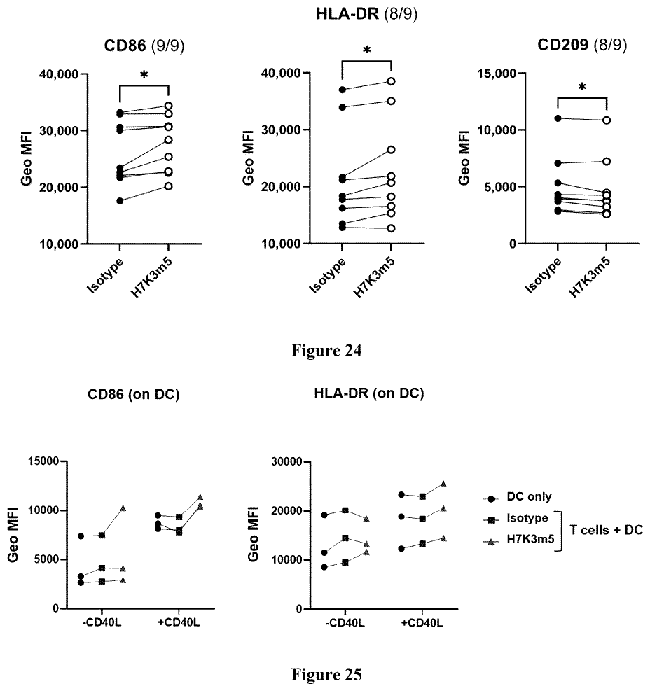

The inventors then tested the effect of H7K3m5 on the dendritic cells differentiated from monocytes (Mo-DC). Classical monocytes were isolated from healthy donor PBMC and differentiated into immature dendritic cells with DC media (StemXVivo, 50 g/mL gentamycin, 50 ng/mL GM-CSF and 35 ng/mL IL-4) for 6 days. The immature monocyte-derived dendritic cells (Mo-DC) were then incubated with antibodies (100 nM) in the presence of 100 ng/mL LPS (TLR4 agonist) to induce dendritic cell maturation and activation. After 2 days, the cells were analyzed by flow cytometry for expression of cell surface markers. As shown in FIG. 24, the increased expression of HLA-DR and of the co-stimulatory molecule CD86 and the decreased expression of the tolerogenic marker CD209 indicate that H7K3m5 enhances the antigen-presentation and pro-inflammatory capacity of Mo-DC in response to TLR signaling.

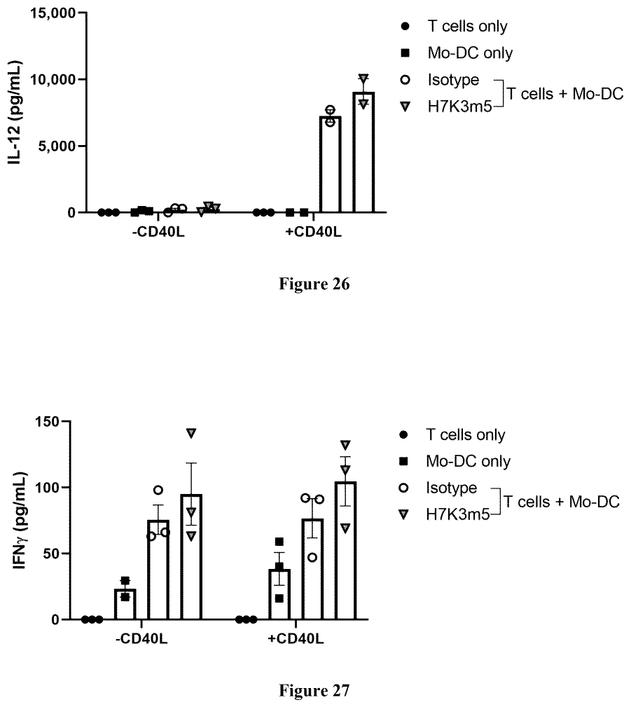

Immature dendritic cells were differentiated from monocytes (Mo-DC) isolated from PBMC of healthy donors with DC media (StemXVivo, 50 g/mL gentamycin, 100 ng/mL GM-CSF and 35 ng/mL IL-4) for 5 days. On day 5, Mo-DC were supplemented with fresh DC media and treated with 30 g/mL H7K3m5 or its isotype control in the absence or presence of g/mL CD40 ligand for an additional 2 days. On day 7, T cells were isolated from PBMC of healthy, unrelated donors and suspended in fresh media containing the cytokine cocktail and g/mL H7K3m5. Cultures of T cells only and Mo-DC only were included as controls. At the end of 4 days, IFN-7 and IL-12 levels in media supernatant were measured by ELISA, whereas the cell surface phenotype of Mo-DC was analyzed by flow cytometry. The results of the experiments illustrated in FIGS. 24-27 show that the pro-inflammatory effect of H7K3m5 on Mo-DC is more pronounced if the latter have been matured with CD40 ligand.

### Example 5

This example illustrates the generation of LILRB4/CD3 bispecific antibodies based on the heavy and light chain variable domain sequences of H7K3m5 and the antibodies disclosed in WO2019057099. Heavy chain heterodimerization was controlled by knob and hole mutations (Merchant et al Nature Biotech 1998, 16, 677-681) stabilized by engineered disulfide bonds (Carter J Immunol Methods 2001, 248, 7-15). Correct light chain pairing was controlled by replacing light chain Ckappa or heavy chain CH1 with human T-cell receptor alpha (TRAC) and beta (TRBC) constant domains as described previously (WO2019057122A1). Mutations were also introduced into the human IgG1 constant domains to reduce effector function, improve stability, and increase productivity in CHO (Alegre et al Transplantation 1994, 57 1537-43 and Hu et al Biotechnol Prog 2017, 33, 786-794).

### Expression and Purification of LILRB4/CD3 Bispecific Antibodies

FIG. 28A shows the schematic representation of 6 first generation LILRB4/CD3 bispecific antibodies in 1+1 or 2+1 configurations. The bispecific antibodies in the 1+1 configurations (4-3ab and 4ab-3) were engineered to bind to a single copy of CD3 epsilon and LILRB4. The bispecific antibodies in the 2+1 (44-4ab, 4ab4ab-3, 43ab-4, and 4ab3-4ab) configurations were engineered to bind to a single copy of CD3 epsilon and 2 copies of LILRB4. The 44-3ab and 4ab4ab-3 configurations have both LILRB4 binding moieties in tandem on one arm of the bispecific. The 43ab-4 and 4ab3-4ab configurations have LILRB4 binding moieties on both arms of the bispecific. The polypeptide chains for each first-generation bispecific antibodies and the amino acid sequences thereof are listed in Table 9.

TABLE 9
Sequences for first-generation LILRB4/CD3 bispecific antibodies

| Antibody | Bispecific | Polypeptide | | SEQ ID |
|---|---|---|---|---|
| No. | Name | Names | Configuration | NO. |
| 1 | 4-3ab | 2 | V_{H}(CD3)-TCRβ-C_{H}2-C_{H}3(KiHa) | 92 |
| | | 3 | V_{H}(LILRB4)-C_{H}1-C_{H}2-C_{H}3(KiHb) | 94 |
| | | 10 | V_{L}(CD3)-TCRα | 101 |
| | | 11 | V_{L}(LILRB4)-C_{L} | 103 |
| 2 | 4ab-3 | 1 | V_{H}(CD3)-C_{H}1-C_{H}2-C_{H}3(KiHa) | 91 |
| | | 5 | V_{H}(LILRB4)-TCRβ-C_{H}2-C_{H}3(KiHb) | 96 |
| | | 9 | V_{L}(CD3)-C_{L} | 100 |
| | | 12 | V_{L}(LILRB4)-TCRα | 104 |
| 3 | 44-3ab | 2 | V_{H}(CD3)-TCRβ-C_{H}2-C_{H}3(KiHa) | 92 |
| | | 4 | V_{H}(LILRB4)-C_{H}1-L-V_{H}(LILRB4)-C_{H}1-C_{H}2-C_{H}3(KiHb) | 95 |
| | | 10 | V_{L}(CD3)-TCRα | 101 |
| | | 11 | V_{L}(LILRB4)-C_{L} | 103 |
| 4 | 4ab4ab-3 | 1 | V_{H}(CD3)-C_{H}1-C_{H}2-C_{H}3(KiHa) | 91 |
| | | 6 | V_{H}(LILRB4)-TCRβ-L-V_{H}(LILRB4)-TCRβ-C_{H}2-C_{H}3(KiHb) | 97 |
| | | 9 | V_{L}(CD3)-C_{L} | 100 |
| | | 12 | V_{L}(LILRB4)-TCRα | 104 |
| 5 | 43ab-4 | 8 | V_{H}(LILRB4)-C_{H}1-L-V_{H}(CD3)-TCRβ-C_{H}2-C_{H}3(KiHa) | 99 |
| | | 3 | V_{H}(LILRB4)-C_{H}1-C_{H}2-C_{H}3(KiHb) | 94 |
| | | 10 | V_{L}(CD3)-TCRα | 101 |
| | | 11 | V_{L}(LILRB4)-C_{L} | 103 |
| 6 | 4ab3-4ab | 7 | V_{H}(LILRB4)-TCRβ-L-V_{H}(CD3)-C_{H}1-C_{H}2-C_{H}3(KiHa) | 98 |
| | | 5 | V_{H}(LILRB4)-TCRβ-C_{H}2-C_{H}3(KiHb) | 96 |
| | | 9 | V_{L}(CD3)-C_{L} | 100 |
| | | 12 | V_{L}(LILRB4)-TCRα | 104 |

To generate the first generation the bispecific antibodies, DNA encoding the first-generation bispecific antibodies were cloned into mammalian expression vectors after gene synthesis. Bispecific antibodies were then expressed by transiently transfecting appropriate mix of vectors into Expi293 cells and expressing in 100 mL scale. All samples were first purified from supernatants by protein A affinity chromatography. Bispecific antibodies No. 1-3 were further purified by size-exclusion chromatography (SEC) while bispecific antibodies 4-6 were purified by anion exchange chromatography (AEX). All samples were analyzed by SDS-PAGE and analytical SEC for purity. The results are shown in Table 10. Aliquots were deglycosylated with PNGase F (MVEDNA Bio M3 103) and were characterized by mass spectrometry using a Water Acquity UPLC coupled to a Xevo G2-XS QTOF using an Acquity UPLC protein BEH SEC column. The results are shown in Table 11.

TABLE 10
Yields and purities of first-generation bispecific antibodies

| | Yield from 100 mL | |
|---|---|---|
| Bispecific Antibody | Expi239 (mg) | Purity (SEC) |
| 4-3ab | 11.4 | 99.5% |
| 4ab-3 | 6.1 | 100% |
| 44-3ab | 5.8 | 96.5% |
| 4ab4ab-3 | 5.1 | 96.0% |
| 43ab-4 | 2.8 | 98.4% |
| 4ab3-4ab | 2.2 | 97.8% |

TABLE 11
Mass spectrometry data for first generation bispecific antibodies

| | Molecular | Molecular | |
|---|---|---|---|
| | Weight | Weight | |
| Bispecific | Calculated | Measured | |
| Antibody | (Da) | (Da) | Significant impurities |
| 4-3ab | 148,640 | 148,626 | Hole-hole homodimer, |
| | | | O-glycan (+948), |
| | | | LC mispairing, |
| | | | pyro-Q (−17) |
| 4ab-3 | 148,640 | 148,266 | Hole-hole homodimer, |
| | | | O-glycan (+948), |
| | | | pyro-Q (−17) |
| 4ab4ab-3 | 199,311 | 199,298 | O-glycan (+948) |
| 4ab3-4ab | 199,227 | 199,230 | None |
| 44-3ab | 196,423 | 196,410 | Pyro-Q (−17) |
| 43ab-4 | 196,423 | 196,427 | None |

Second generation bispecific antibodies were designed to improve homogeneity and manufacturability. Specifically, this was done by mutating S91A in the TCRα domain to remove O-glycan modification or making a Q1E mutation to prevent N-terminal pyro-Q formation. The polypeptide chains for each first-generation bispecific antibodies and the amino acid sequences thereof are listed in Table 12. DNA encoding the second-generation bispecific antibodies and controls were cloned into mammalian expression vectors after gene synthesis or standard molecular biology protocols starting from first-generation bispecific antibodies. Bispecific antibodies were then expressed using appropriate vectors transiently transfected into CHO-K1 cells and expressed at 1 L scale for 14 days using a fed-batch protocol. Bispecific antibodies were purified from supernatant from all samples using by a protein A column and polished by SEC. All samples were analyzed by SDS-PAGE and analytical SEC for purity. The results are shown in Table 13. Aliquots of samples were deglycosylated and were characterized by mass spectrometry, which showed that the samples lacked impurities of first-generation bispecific antibodies (see Table 14).

TABLE 12
Sequences for Second Generation LILRB4/CD3 Bispecific antibodies

| | | | | SEQ ID |
|---|---|---|---|---|
| Name | Description | Polypeptide | Configuration | NO. |
| 4ab3-4ab | Bispecific 1 | 7 | V_{H}(LILRB4)-TCRβ-L-V_{H}(CD3)-C_{H}1-C_{H}2-C_{H}3(KiHa) | 98 |
| | | 5 | V_{H}(LILRB4)-TCRβ-C_{H}2-C_{H}3(KiHb) | 96 |
| | | 9 | V_{L}(CD3)-C_{L} | 100 |
| | | 12 S91A | V_{L}(LILRB4)-TCRα(S91A) | 105 |
| 4-3ab Q1E | Bispecific 2 | 2 Q1E | V_{H}(CD3)-TCRβ-C_{H}2-C_{H}3(KiHa) | 93 |
| S91A | | | (Q1E) | |
| | | 3 | V_{H}(LILRB4)-C_{H}1-C_{H}2-C_{H}3(KiHb) | 94 |
| | | 10 S91A | V_{L}(CD3)-TCRα(S91A) | 102 |
| | | 11 | V_{L}(LILRB4)-C_{L} | 103 |
| anti-LILRB4 | Control 1 | 13 LALA | V_{H}(LILRB4)-C_{H}1-C_{H}2-C_{H}3 | 106 |
| IgG1 LALA | | 11 | V_{L}(LILRB4)-C_{L} | 103 |
| anti-CD3 | Control 2 | 1 | V_{H}(CD3)-C_{H}1-C_{H}2-C_{H}3(KiHa) | 91 |
| IgG1 LALA | | 9 | V_{L}(CD3)-C_{L} | 100 |
| | | 14 | C_{H}2-C_{H}3(KiHb) | 107 |

TABLE 13
Yields and purities of second-generation bispecific antibodies

| | Yield from 1 L | |
|---|---|---|
| Name | CHO-K1 (mg) | Purity (SEC) |
| 4ab3-4ab S91A | 164.82 | 98.6% |
| 4-3ab Q1E S91A | 205.56 | 98.4% |
| anti-LILRB4 IgG1 | 416.50 | 99.6% |
| LALA | | |
| anti-CD3 IgG1 LALA | 38.52 | 99.6% |

TABLE 14
Mass spectrometry data for second generation bispecific antibodies

| | | | Significant |
|---|---|---|---|
| Name | Calculated (Da) | Measured (Da) | impurities |
| 4ab3-4ab S91A | 199192.82 | 199190.7 | None |
| 4-3ab Q1E S91A | 148623.13 | 148621.8 | None |
| anti-LILRB4 IgG1 | 145027.37 | 145027.8 | None |
| LALA | | | |
| anti-CD3 IgG1 | 98523.88 | 98529.8 | Pyro-Q |
| LALA | | | |

### In Vitro Cytotoxicity Assay

A FACS based approach was used to determine the ability of the LILRB4/CD3 bispecific antibodies to mediate target cell killing by T cells. In vitro cytotoxicity of THP-1 and MV4-11 cells: Human AML cell line THP-1 and MV-4-11 cells were engineered to express green fluorescent protein (GFP). Human buffy coats were obtained from heathy donors collected by Stanford Blood Center. Human peripheral blood mononuclear cells (PBMC) were isolated from buffy coats by Ficoll Paque Plus (GE Healthcare Catalog No. 17-1440-03) density gradient cell separation. Pan T cells were further isolated from PBMCs using a human Pan T cell isolation kit (Miltenyi Biotec Catalog No. 130-096-535). 5×10⁵ freshly isolated human pan T cells were used as effector cells and 1×10⁵ THP-1-GFP were used as target cells in a 5:1 ratio. In the MV4-11 cell killing assay, 9×10⁵ freshly isolated human pan T cells were used as effector cells and 1×10⁵ MV4-11-GFP were used as target cells in a 9:1 ratio. Human pan T cells, THP-1-GFP or MV4-11-GFP cells, and increasing concentrations of IO-202 or isotype control human IgG1 (BioXcell Catalog No. BE0297) were mixed in 200 μL total in RPMI 1640 (Gibco Catalog No. 61870-036)+10% heat-inactivated fetal bovine serum (FBS; Gibco Catalog No. 10082-147) in U-shaped 96-well plate and incubated for 48 hrs at 37° C. At the end of incubation, 7-aminoactinomycin D (7-AAD; BD Pharmingen Catalog No. 559925) was added to cells and 100 μL of cells was acquired by FACS Celesta and the percentage of GFP-positive cells were measured. Flow cytometry data were analyzed using Flowjo software (Flowjo LLC) and cell cytotoxicity was calculated as: percent of cytotoxicity=100−([T/NT]×100), where T and NT are the percentages of GFP+ cells treated with or without test antibodies, respectively.

Autologous killing of human normal monocytes: PBMCs at 1×10⁶ and increased concentrations of LILRB4/CD3 bispecific antibodies or controls were mixed in 200 μL total in RPMI+10% heat-inactivated FBS and 50 ng/ml of IL-2 (R&D systems Catalog No. 202-IL/CF) in a U-shaped 96-well plate and incubated for 48 hours at 37° C. PBMCs incubated with increasing concentrations of rituximab (Biogen/Genentech) were used as assay controls. At the end of incubation, cells were washed and incubated with 5 μL of Fc receptor blocker (human immunoglobulin G [IgG] at 10 mg/mL, Sigma Aldrich Catalog No. 14506) at room temperature for 10 minutes, followed by incubation on ice for 30 minutes with 100 μL of fluorescent conjugated CD14 (Clone M5E2, Catalog No. 555397) and CD19 (Clone HIB19, Catalog No. 555415) from BD Biosciences. Monocytes were identified as CD14-positive cells, while B cells were identified as CD19-positive cells.

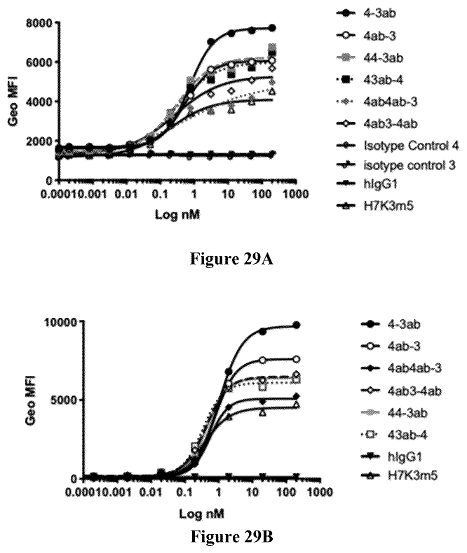

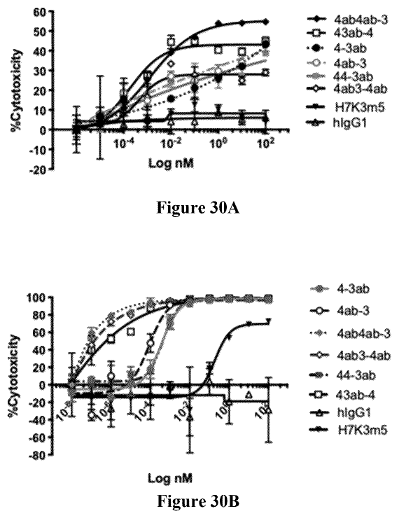

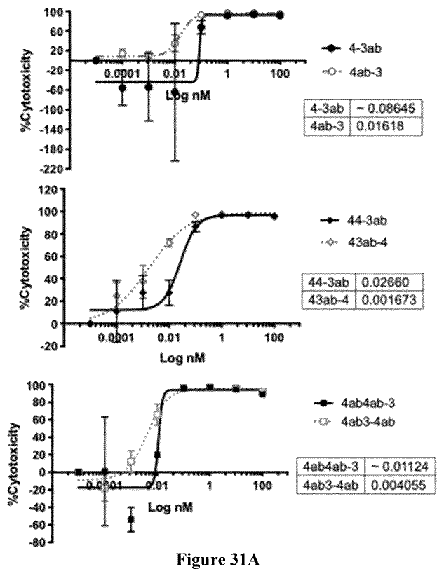

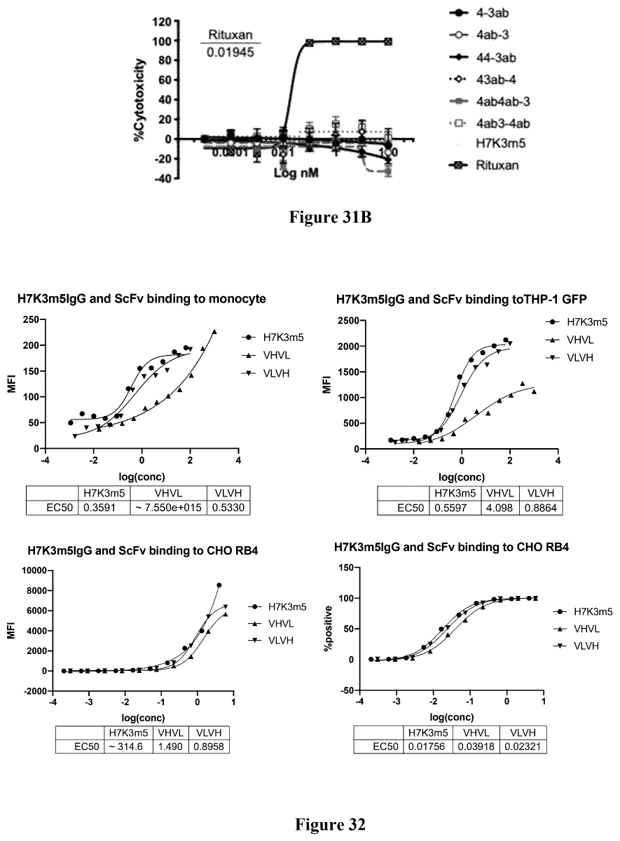

As shown in FIGS. 29A-29B, similar binding affinity trends were observed across different anti-LILRB4 mono-specific and bispecific antibodies between monocytes and THP-1. As shown in FIGS. 30A-30B, T cell-mediated cytotoxicity of bispecific LILRB4/CD3 antibodies on monocytes and THP-1-luc-GFP cells are observed. As shown in FIGS. 31A-31B, autologous killing of monocytes by LILRB4/CD3 bi-specifics (FIG. 31A) and autologous killing of B cells by Rituxan as a control (FIG. 31B) were observed.

Cell killing curve generation and EC₅₀ calculation was performed by Prism GraphPad software using non-linear sigmoidal dose-response curve fit. The average±SD were calculated by Excel. Results are reported in Tables 15 and 16.

TABLE 15
T-cell cytotoxicity of first generation bispecifics

| | THP-1 | | | | | | | |
|---|---|---|---|---|---|---|---|---|
| | EC₅₀ | THP-1 | Monocyte EC₅₀ (nM) | | | Monocyte (max %) | | |
| Name | (nM) | (max %) | Donor 1 | Donor 2 | Donor 3 | Donor 1 | Donor 2 | Donor 3 |
| 4-3ab | 0.00036 | 98 | n/a ** | 0.0094 | no activity | 43 | 41 | 13 |
| 4ab-3 | 0.000084 | 98.6 | n/a ** | 0.029 | 0.00925 | 43 | 34 | 32.5 |
| 44-3ab | 0.00043 | 96.4 | n/a ** | 0.0034 | no activity | 39 | 30 | 0 |
| 4ab4ab-3 | <0.00001* | 98.3 | 0.0013 | 0.00032 | 0.0029 | 55 | 59 | 54.5 |
| 43ab-4 | <0.00001* | 98.7 | 0.00019 | no activity | 0.000084 | 45 | 0 | 42 |
| 4ab3-4ab | <0.00001* | 98.1 | 0.00043 | 0.00093 | 0.0016 | 29 | 44 | 44 |

*estimated EC₅₀
** EC50 cannot be measured accurately; 4-3ab, 4ab-3, and 44-3ab have weaker potency than 4ab4ab-3, 43ab-4, and 4ab3-4ab

TABLE 16
T-cell cytotoxicity of second-generation bispecific antibodies

| | THP-1 | MV4-11 | Monocyte | B-cell |
|---|---|---|---|---|
| Name | EC₅₀ (nM) | EC₅₀ (nM) | EC₅₀ (nM) | EC₅₀ (nM) |
| 4ab3-4ab | 0.00050 | 0.000034 | 0.0059 | no activity |
| 4-3ab | 0.0043 | 0.0025 | 0.026 | no activity |
| anti-LILRB4 | no activity | no activity | no activity | no activity |
| IgG1 LALA | | | | |
| anti-CD3 | not tested | no activity | no activity | no activity |
| IgG1 LALA | | | | |
| Rituxan | not tested | not tested | no activity | 0.043 |

### Surface Plasmon Resonance (Biacore)

Binding affinities of first-generation bispecific antibodies to recombinant CD3e-CD3d heterodimer protein (Acro Biosystems) or LILRB4 recombinant protein (Sino Biological) were measured by surface plasmon resonance using a Biacore 8K instrument. Briefly, CD3e-CD3d or LILRB4 protein was immobilized on a CM5 chip with EDC and NHIS according to standard protocols. Bispecific antibody analytes were then injected at 6 concentration (1.25, 2.5, 5, 10, 20, and 40 nM) or 7 concentrations (20, 2.5, 5, 10, 20, 40, and 80 nM) in 1×HBIX-EP+(10 mM HEPES, 150 mM NaCl, 3 mM EDTA, 0.05% Tween 20, pH 7.4), for LILRB4 and CD3e-CD3d, respectively. The chip was regenerated using 10 mM glycine pH 1.5 as the regeneration buffer. The sensorgrams for reference channel Fc1 and buffer channel were subtracted from the test sensorgrams and the experimental data was fitted by 1:1 binding model. See Table 17 for affinities.

TABLE 17
Affinities of first generation bispecifics
to recombinant protein and cells

| | | Biacore | | |
|---|---|---|---|---|
| | Biacore affinity | Affinity to | FACS EC₅₀ to | FACS EC₅₀ to |
| | to CD3e-CD3d | LILRB4 | CD3+ Jurkat | LILRB4+ |
| Bispecific | (K_{D}, n M) | (K_{D}, nM) | cells | THP-1 cells |
| 4-3ab | 49.6 | 0.19 | NC | 1.3 |
| 4ab-3 | 33.4 | 0.27 | NC | 0.77 |
| 44-3ab | 42.1 | 0.23 | NC | 0.45 |
| 4ab4ab-3 | 26.8 | 0.26 | NC | 0.55 |
| 43ab-4 | 47.0 | 0.29 | NC | 0.33 |
| 4ab3-4ab | 57.3 | 0.26 | NC | 0.43 |

### Bio-Layer Interferometer (Gator Bio)

Binding affinities of second-generation bispecific antibodies were measured to recombinant human CD3e (Acro, cat #CDE-H5223), cyno CD3e (Acro, cat #CDE-C5226), human LILRB4 (Sino Biological, cat #16742-H08H), and cyno LILRB4 (Acro, cat #CDK-C5227) by Bio-Layer Interferometry (BLI) using an instrument from Gator Bio. Bispecific antibodies were captured using an immobilized anti-human Fc antibody (HFC) probe (Gator Bio, cat #PL168-160003) at 5 μg/mL load. Binding affinities were measured using 6 concentrations (0.6 to 159 nM, 0.7 to 164 nM, 0.8 to 200 nM, and 2.5 to 595 nM), for human CD3e, cyno CD3e, human LILRB4, and cyno LILRB4, respectively. Binding affinity constants were determined using a 1:1 fitting model (Global Fit) with Gator's data analysis software 1.6.1.1203, and the KD was calculated using the ratio Kdis/Kon. See Table 18 for affinities.

TABLE 18
Affinities of second-generation bispecific antibodies to recombinant proteins and cells

| | FACS EC₅₀ | Affinity | Affinity | FACS EC₅₀ | Affinity | Affinity |
|---|---|---|---|---|---|---|
| | to CD3 + | to human | to cyno | to LILRB4 + | to human | to cyno |
| | Jurkat cells | CD3e | CD3e | THP-1 cells | LILRB4 | LILRB4 |
| Bispecific | (estimated, nM) | (KD, nM) | (KD, nM) | (nM) | (KD, nM) | (KD, nM) |
| 4ab3-4ab | 256.7 | 0.07 | 0.26 | 0.7 | 0.89 | 8.82 |
| S91A | | | | | | |
| 4-3ab | 853.6 | 0.10 | 0.07 | 1.3 | 0.84 | 9.53 |
| Q1E/S91A | | | | | | |

### Flow Cytometry

Binding of first and second generation bispecifics to CD3 or LILRB4 was measured by FACS on Jurkat cells or THP-1 cells. To measure binding of bispecifics to THP-1 cells, THP-1 cells were first incubated with human Fc blocker (BD Pharmingen Catelog No. 564220) at 10 μg per one million cells at room temperature for 10 minutes, followed by incubated with serially diluted bispecifics for 30 minutes on ice. Cells were washed twice with BSA staining buffer (BD Pharmingen Catelog No. 554657) and incubated with secondary Alexa 647 conjugated anti-human IgGFc monoclonal antibody (Biolegend Catelog No. 409306) at 5 μg/mL for 30 minutes on ice. After final wash, 7-AAD was applied to exclude dead cells. To measure binding of first generation bispecifics to Jurkat cells, each bispecific was diluted in buffer including 1% BSA (5-fold dilution series, 400 nM highest concentration) and incubated with cells for 30 minutes at 4° C. Then bispecifics were stained with Alexa 647 anti-human IgG Fc at 5 μg/mL at 4° C. for 0.5 hours before analysis. Binding for CD3 was clearly observed on the cells compared to an isotype control, but the EC50 could not be calculated or only estimated since the binding curve did not reach a plateau. See Tables 17 and 18 for EC50 values.

### Example 6

This example illustrates the generation of CAR-T cells that express a CAR protein based on the heavy chain and light chain variable region sequences of H7K3m5.

Two different configurations of single chain Fv (scFv) derived from anti-LILRB4 monoclonal antibody H7K3m5 were tested for binding to human primary monocytes and human leukemia cell THP-1 using flow cytometry. As shown in FIG. 32, the VlVh configuration maintains the binding affinity as H7K3m5 whereas the VhVl configuration lost some binding affinity. Therefore, VlVh is selected for the CAR constructs.

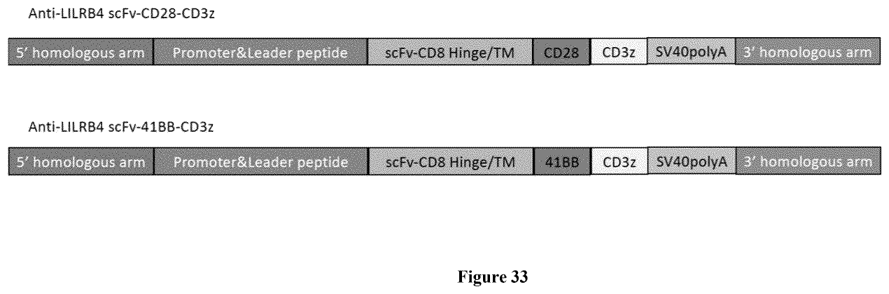

As illustrated in FIG. 33, the DNA construct for expressing the anti-LILRB4 CAR proteins is a 2^{nd} generation CAR constructs containing CD28 or 4-1BB costimulatory domains with CD3zeta activation domain. The scFv is derived from anti-LILRB4 monoclonal antibody H7K3m5. The 5′ and 3′ homologous arms are homologous sequences upstream and downstream of the Cas9 DNA cleavage site in TRAC gene (based on gRNA design). Promoter and leader peptide are necessary elements for gene expression and extracellular translocation. Here the inventors used JeT promoter to control the expression of scFv (Eyquem J et al. Nature (2017) 543:113-117). SV40 poly-A tail is included for improving transcript stability and translation.

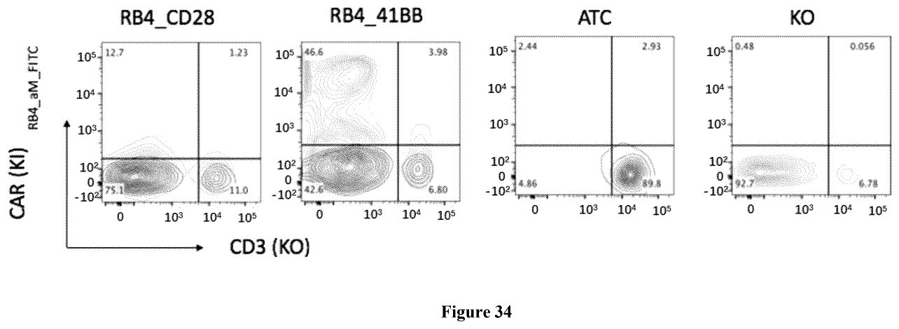

Human primary T cells were transfected with CRISPR-Cas9 RNP complexes including a guide RNA targeting the 5′ end of the first exon of TRAC and supplied with the DNA construct for homologous recombination-based knock in. After knocking out TCRalpha, the cells were grown in complete Optimizer medium with IL-2 300 IU/ml and without anti-CD3/28 added. Following transfection, cells were expanded in culture for 2 weeks. Anti-LILRB4 CAR-T cells were identified by binding to LILRB4-Fc fusion protein (ACRObiosystems CDK-H5259) and anti-Fc antibody (Biolegend B278652). Success in knock-out of the endogenous TCR alpha (TRAC) locus (KO) was measured by anti-CD3 staining (anti-CD3 PE, BD 555333). As shown in FIG. 34, efficient generation of anti-LILRB4 CAR-T cells was confirmed.

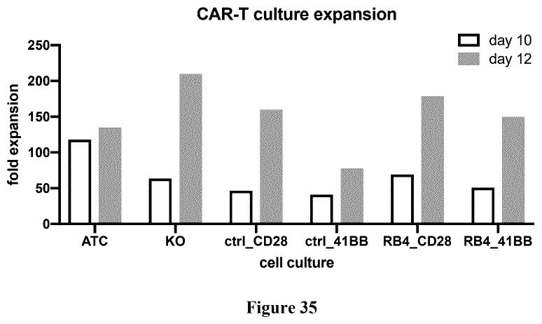

As shown in FIG. 35, the TCR alpha (TRAC) inactivated T cells (KO) and anti-LILRB4 CAR (or control CAR) knocked-in T cells undergo cell proliferation and expansion in vitro. Anti-LILRB4 CAR-T cells had significantly higher fold of expansion in comparison to the control CAR-T cells.

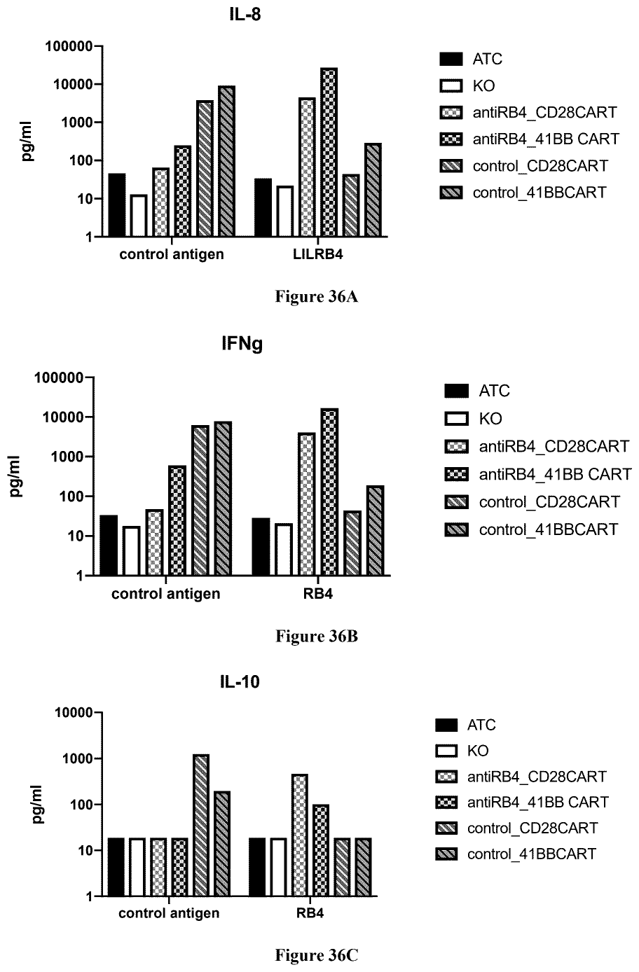

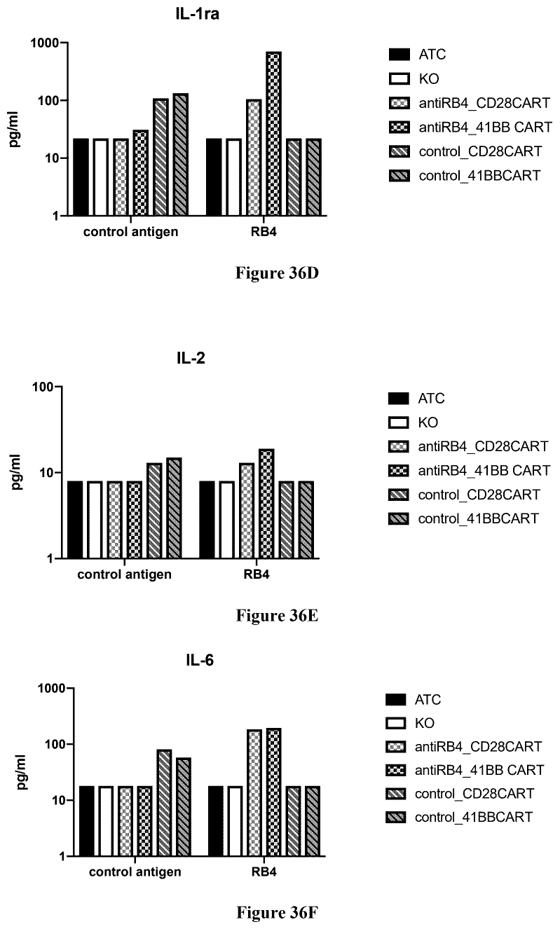

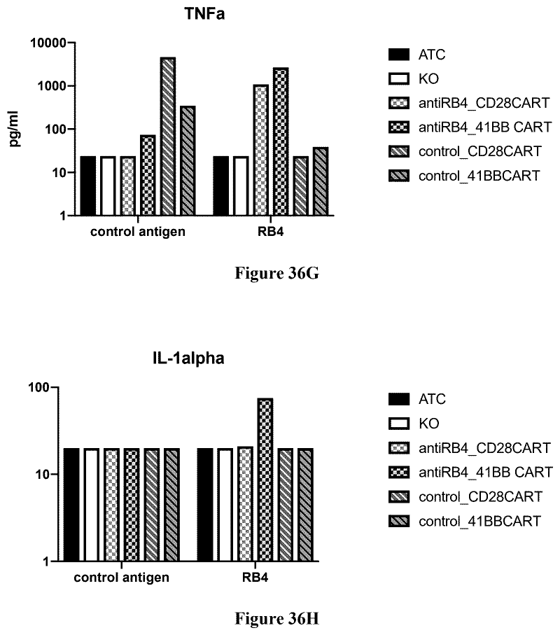

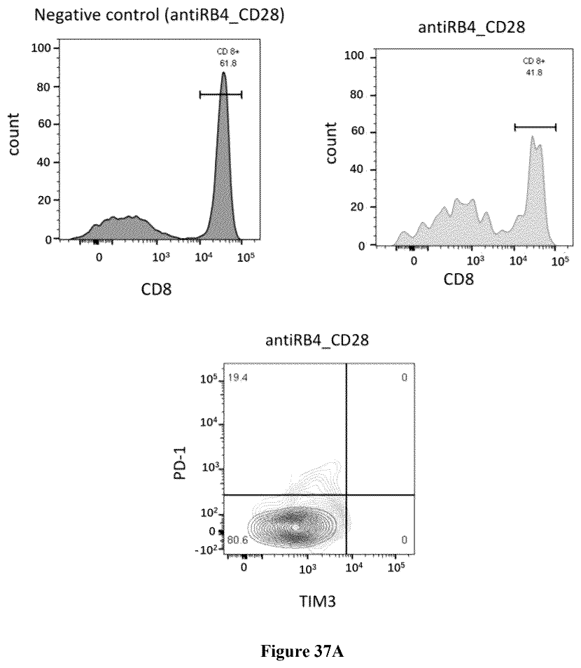

To test the antigen-dependent activation of CAR-T culture, 1 μg/ml recombinant control antigen or LILRB4 antigen were coated on 96 well plate overnight in PBS buffer. Plates were washed twice with PBS buffer. 1×10⁵ CAR-T cells in culture media (without any cytokine added) were added to each well and incubated for 72 hours. Cell culture supernatant were collected for cytokine release measurement by Luminex assay. As shown in FIGS. 36A-36H, the release of cytokine IL-8, IFNγ, IL-10, IL-1ra, IL-2, IL-6, TNFα and IL1alpha by the anti-LILRB4 CAR-T cells depends on the presence of LILRB4.

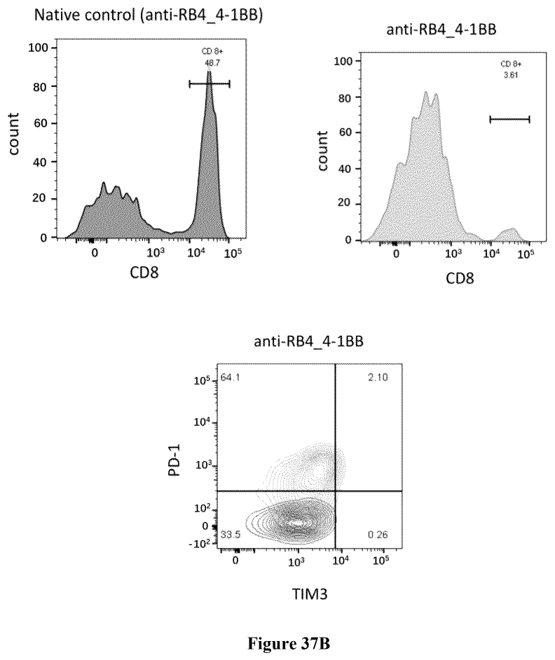

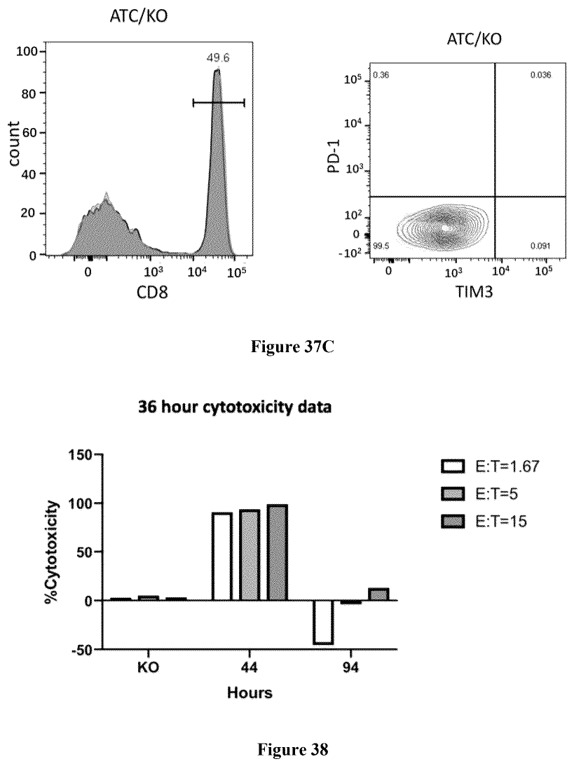

FIGS. 37A-37C show the characterization of CAR-T cells after 2 weeks expansion. Frozen CAR-T cells in liquid nitrogen storage were thawed and kept in culture for 2-3 days before flow cytometry analysis. Antibodies used were anti-CD8 APC Cy7 (BD561945), anti-PD1 PE (BD560908) anti-TIM3 BV421 (BD565562). LILRB4 CAR-T cells were identified by binding to LILRB4-Fc fusion protein (ACRObiosystems CDK-H5259) and anti-Fc antibody (Biolegend B278652). As shown in FIG. 37A, expressing anti-LILRB4_CD28 slightly decreased the percentage of CD8+ T cells (from 61.8% to 41.8%) and did not substantially change the expression of PD-1 and TIM3 on the cells. As shown in FIG. 37B, expressing anti-LILRB4_4-1BB decreased the percentage of CD8+ T cells from 48.7% to 3.61% and increased the expression of PD-1. As shown in FIG. 37C, knock-out (KO) TCR alpha expression at TRAC locus, where the CAR construct was inserted, did not substantially change the percentage of CD8+ T cells or the expression of PD-1 or TIM3.

To test the cytotoxicity of the anti-LILRB4 CAR-T cells, CHO K1 RB4 cells were seeded at different density (6×10⁴, 2×10⁴ or 7×10³) for 12 hours, 1×10⁵ CAR-T cells were added and cytotoxicity were measured by removing the supernatant CAR-T cells and wash the plate 2 times with PBS. Total viable adherent CHO K1 RB4 cells were measured by Promega CTG2.0 luminescence kit, and the % cytotoxicity were calculated by dividing the Luminescent signal of each condition with the same E:T ratio activated T cell control. As shown in FIG. 38, anti-LILRB4 CAR-T cells (44) but not anti-CD19 CAR-T cells (94) killed CHO K1 RB4 cells.

### Example 7

This example illustrates the design of phase 1 First-in-Human trial.

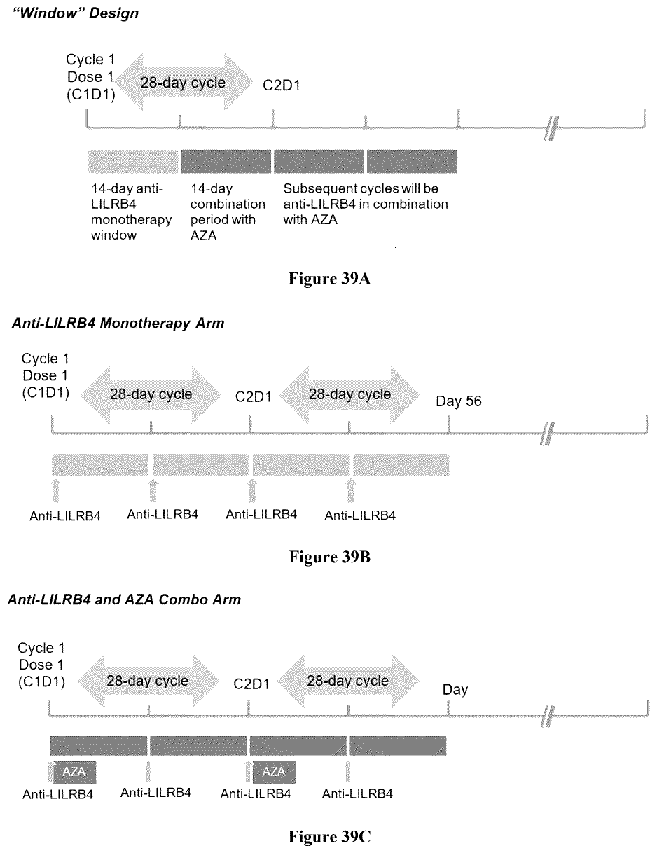

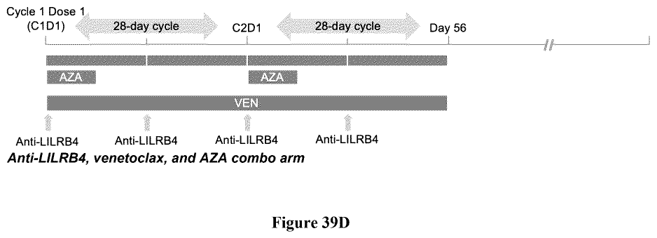

As shown in FIG. 39A, a “window” design comprising of a 2-week monotherapy lead-in (“window”) of anti-LILRB4 enables the studying of the effects of a monoclonal antibody that specifically targets LILRB4 as monotherapy.

During the Part 1A monotherapy escalation phase (single dose on Day 1) patients will be enrolled into sequential cohorts of increasing doses of anti-LILRB4 monotherapy. The goal of Part 1A is to determine the MTD of anti-LILRB4 monotherapy (MTD1). DLTs for MTD1 will be evaluated during the first 14 days of treatment (prior to the first dose of anti-LILRB4 in combination with azacytidine/azacitidine), i.e., the first dose-interval for anti-LILRB4. The initial dose-escalation begins with an accelerated titration design followed by a standard escalation phase that will use a 3+3 design. Part 1 will include both relapsed and/or refractory myelomonocytic (M4) and monocytic/monoblastic (M5) AML patients and chronic myelomonocytic leukemia (CMML) patients.

During Part 1B (starting on Day 15), patients without DLTs during Part 1A will receive the same dose of anti-LILRB4 that was administered in Part 1A in combination with a standard dose of azacytidine/azacitidine (75 mg/m² subcutaneously for 7 days every 28 days). The MTD of anti-LILRB4 in combination with azacytidine/azacitidine (MTD2) will be determined in the 28-day DLT window, consisting of the 14 days of monotherapy and 14 days of the combination treatment.

The overall DLT period of Part 1 (Part 1A and Part 1B combined) is 28 days. This could also be easily changed to 42 days, with the first 14 days for monotherapy DLT and the last 28 days (with addition of azacytidine/azacitidine) for combotherapy DLT.

Subsequent cycles will be anti-LILRB4 in combination with azacytidine/azacitidine.

As shown in FIG. 39B and FIG. 39C, once the MTD and/or RP2D for the anti-LILRB4+ azacytidine/azacitidine combination has been identified and approval given by the Safety Review Committee (SRC), enrollment in one of two expansion arms (FIG. 39B and FIG. 39C) will commence in relapsed and/or refractory monoblastic/monocytic leukemia patients.

As shown in FIG. 39B, this study will enroll a monotherapy cohort anti-LILRB4 in patients with relapsed/refractory AML with monocytic differentiation. FIG. 39B can also be the design for a First-in-Human phase 1 trial in case the “window” designed is not accepted.

As shown in FIG. 39C, this study will enroll a combination cohort of anti-LILRB4+ azacytidine/azacitidine in patients with relapsed/refractory AML with monocytic differentiation.

As shown in FIG. 39D, this study will enroll a combination cohort of anti-LILRB4+ azacytidine/azacitidine+venetoclax in patients with relapsed/refractory AML with monocytic differentiation and in patients with newly diagnosed AML with monocytic differentiation.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A bispecific antibody or antigen-binding fragment thereof capable of binding to Leukocyte Immunoglobulin-like subfamily B member 4 (LILRB4) and CD3, comprising:
(a) a first antigen-binding region capable of binding to LILRB4 comprising a first light chain variable (V_{L}) domain and a first heavy chain variable (V_{H}) domain; and
(b) a second antigen-binding region capable of binding to CD3 comprising a second V_{L} domain and a second V_{H} domain,
wherein
(i) the first V_{H} domain comprising a first HC-CDR1 having the amino acid sequence of SEQ ID NO: 5, a first HC-CDR2 having the amino acid sequence of SEQ ID NO: 6 and a first HC-CDR3 having the amino acid sequence of SEQ ID NO: 7; and
(ii) a first V_{L} domain comprising a first LC-CDR1 having the amino acid sequence of SEQ ID NO: 8 with a mutation at amino acid residues NS, a first LC-CDR2 having the amino acid sequence of SEQ ID NO: 9 and a first LC-CDR3 having the amino acid sequence of SEQ ID NO: 10.

2. The bispecific antibody or antigen-binding fragment thereof of claim 1, wherein the LC-CDR1 having the amino acid sequence of SEQ ID NO: 28.

3. The bispecific antibody or antigen-binding fragment thereof of claim 1, wherein the first V_{H} domain has the amino acid sequence of SEQ ID NO: 1; and wherein the first V_{L} domain has the amino acid sequence of SEQ ID NO: 27.

4. The bispecific antibody or antigen-binding fragment of claim 1, wherein the first V_{L} domain and the first V_{H} domain link to a first pair of constant domains, respectively, and wherein the second V_{L} domain and the second V_{H} domain link to a second pair of constant domains, respectively.

5. The bispecific antibody or antigen-binding fragment of claim 4, wherein
(a) the first V_{L} domain links to a first light chain constant (C_{L}) domain, and the first V_{H} domain links to a first heavy chain constant domain 1 (C_{H}1), or
(b) the first V_{L} domain links to a first C_{H}1 domain, and the V_{H} domain links to a second C_{L} domain.
(c) the first V_{L} domain links to a T cell receptor (TCR) α chain constant domain, and the first V_{H} domain links to a TCR β chain constant domain, or
(d) the first V_{L} domain links to a TCR β chain constant domain, and the first V_{H} domain links to a TCR α chain constant domain.

6. The bispecific antibody or antigen-binding fragment of claim 4, wherein
(a) the second V_{L} domain links to a second CL domain, and the second V_{H} domain links to a second C_{H}1 domain, or
(b) the second V_{L} domain links to a second C_{H}1 domain, and the second V_{H} domain links to a second CL domain, or
(c) the second V_{L} domain links to a T cell receptor (TCR) α chain constant domain, and the second V_{H} domain links to a TCR β chain constant domain, or
(d) the second V_{L} domain links to a TCR β chain constant domain, and the second V_{H} domain links to a TCR α chain constant domain.

7. The bispecific antibody or antigen-binding fragment of claim 6, wherein the TCR α chain constant domain has a S91A mutation.

8. The bispecific antibody or antigen-binding fragment of claim 1, wherein the first antigen-binding region and/or the second antigen-binding region is a single chain variable fragment (scFv).

9. The bispecific antibody or antigen-binding fragment of claim 1, further comprising a third antigen-binding region comprising a third V_{L} domain and a third V_{H} domain, wherein the third antigen-binding region is capable of binding to LILRB4 or CD3.

10. The bispecific antibody or antigen-binding fragment of claim 9, wherein the third V_{L} domain and the third heavy chain variable domain link to a first pair of constant domains, respectively.

11. The bispecific antibody or antigen-binding fragment of claim 10, wherein
(a) the third V_{L} domain links to a third CL domain, and the third V_{H} domain links to a third C_{H}1 domain, or
(b) the third V_{L} domain links to a third C_{H}1 domain, and the third V_{H} domain links to a third C_{L} domain, or
(c) the third V_{L} domain links to a TCR α chain constant domain, and the third V_{H} domain links to a TCR β chain constant domain, or
(d) the third V_{L} domain links to a TCR β chain constant domain, and the third V_{H} domain links to a TCR α chain constant domain.

12. The bispecific antibody or antigen-binding fragment thereof of claim 1, which is linked to one or more conjugate moieties.

13. The bispecific antibody or antigen-binding fragment thereof of claim 12, wherein the conjugate moiety comprises a clearance-modifying agent, a toxin, a detectable label, a chemotherapeutic agent, a cytokine, or purification moiety.

14. A pharmaceutical composition comprising the bispecific antibody or antigen-binding fragment thereof of claim 1, and a pharmaceutically acceptable carrier.

15. An isolated polynucleotide encoding the bispecific antibody or antigen-binding fragment thereof of claim 1.

16. A vector comprising the isolated polynucleotide of claim 15.

17. A host cell comprising the vector of claim 16.

18. A method of expressing a bispecific antibody or antigen-binding fragment thereof capable of binding to LILRB4 and CD3, comprising culturing the host cell of claim 17 under the condition at which the bispecific antibody or antigen-binding fragment is expressed.

19. A method of treating or ameliorating the effect of a cancer in a subject, comprising administering to the subject a therapeutically effective amount of the bispecific antibody or antigen-binding fragment thereof of claim 1.

20. The method of claim 19, wherein the cancer is selected from the group consisting of multiple myeloma (MM), blastic plasmacytoid dendritic cell neoplasm (BPDCN), lymphoma, lymphocytic leukemia, Hodgkin's Disease, acute myeloid leukemia (AML), acute lymphocytic/lymphoblastic leukemia, chronic lymphocytic leukemia, chronic myeloid leukemia (CML), myelodysplastic syndrome (MDS), myeloproliferative neoplasms, and chronic myelomonocytic leukemia (CMML).

21. The method of claim 19, wherein the subject is human.

22. The method of claim 19, wherein the bispecific antibody or an antigen-binding fragment thereof is administered intravenously, intra-arterially, intra-tumorally, or subcutaneously.

23. A method for treating or ameliorating an autoimmune or inflammatory disease in a subject, comprising administering to the subject a therapeutically effective amount of the bispecific antibody or antigen-binding fragment thereof of claim 1.

24. A method of killing a cell in a subject, comprising administering to the subject the bispecific antibody or antigen-binding fragment thereof of claim 1.

25. A method of activating a T cell, comprising contacting the T cell with the bispecific antibody or an antigen-binding fragment thereof of claim 1, optionally in the presence of a target cell expressing LILRB4.


SEQUENCE LISTING (124 sequences)

SEQ ID NO: 1 - protein, 120 aa, synthetic construct
  REGION 1..120: note = Synthetic
EVQLVESGGGLVQPGGSLRLSCAASGFSLSSSYWISWVRQAPGKGLEWIGSIDSGSVGITYYATWVKGRFTISRDNSKNTLYLQMNSLRAEDTAVYYCARHGDNWALDLWGQGTLVTVSS

SEQ ID NO: 2 - DNA, 360 bp, synthetic construct
  misc_feature 1..360: note = Synthetic
  source 1..360: mol_type = other DNA
gaggtgcagctggtcgagtccggaggcggactggtgcagcctggaggatccctgaggctgtcctgcgccgcttccggcttctccctgtcctcctcctactggatctcctgggtgaggcaggcccctggaaagggcctggagtggatcggctccatcgactccggctccgtgggcatcacctactacgccacctgggtgaagggcaggttcaccatctccagggacaactccaagaacaccctgtacctgcagatgaactccctgagggccgaggacaccgccgtgtactactgcgccaggcacggcgataactgggctctggacctgtggggacagggcacactggtgaccgtgtcctcc

SEQ ID NO: 3 - protein, 110 aa, synthetic construct
  REGION 1..110: note = Synthetic
DIQMTQSPSTLSASVGDRVTITCRASQSINSWLAWYQQKPGKAPKLLIYKASTLASGVPSRFSGSGSGTEFTLTISSLQPDDFATYYCQHGYIRGDLDNVFGGGTKVEIK

SEQ ID NO: 4 - DNA, 330 bp, synthetic construct
  misc_feature 1..330: note = Synthetic
  source 1..330: mol_type = other DNA
gacatccagatgacccagtccccttccaccctgtccgcttccgtgggcgacagggtgaccatcacctgcagggccagccagtccatcaactcctggctggcctggtaccagcagaagcccggcaaggcccccaagctgctgatctacaaggcttccaccctggcttccggcgtgcctagcaggttttccggctccggctccggcaccgagtttaccctgaccatctcctccctgcagcccgacgacttcgccacctactactgccagcacggctacatcaggggcgacctggacaacgtgtttggcggcggcaccaaggtggagatcaag

SEQ ID NO: 5 - protein, 9 aa, synthetic construct
  REGION 1..9: note = Synthetic
GFSLSSSYW

SEQ ID NO: 6 - protein, 9 aa, synthetic construct
  REGION 1..9: note = Synthetic
IDSGSVGIT

SEQ ID NO: 7 - protein, 11 aa, synthetic construct
  REGION 1..11: note = Synthetic
ARHGDNWALDL

SEQ ID NO: 8 - protein, 6 aa, synthetic construct
  REGION 1..6: note = Synthetic
QSINSW

SEQ ID NO: 9 - protein, 7 aa, synthetic construct
  REGION 1..7: note = Synthetic
KASTLAS

SEQ ID NO: 10 - protein, 11 aa, synthetic construct
  REGION 1..11: note = Synthetic
HGYIRGDLDNV

SEQ ID NO: 11 - protein, 120 aa, synthetic construct
  REGION 1..120: note = Synthetic
EVQLVESGGGLVQPGGSLRLSCAASGFSLSSSYWISWVRQAPGKGLEWIGSIDSGSVGITYYATWVKGRFTISRDNSKNTLYLQMNSLRAEDTAVYYCARHGDNVALDLWGQGTLVTVSS

SEQ ID NO: 12 - protein, 11 aa, synthetic construct
  REGION 1..11: note = Synthetic
ARHGDNVALDL

SEQ ID NO: 13 - protein, 120 aa, synthetic construct
  REGION 1..120: note = Synthetic
EVQLVESGGGLVQPGGSLRLSCAASGFSLSSSYWISWVRQAPGKGLEWIGSIDSGSVGITYYATWVKGRFTISRDNSKNTLYLQMNSLRAEDTAVYYCARHGDNYALDLWGQGTLVTVSS

SEQ ID NO: 14 - protein, 11 aa, synthetic construct
  REGION 1..11: note = Synthetic
ARHGDNYALDL

SEQ ID NO: 15 - protein, 120 aa, synthetic construct
  REGION 1..120: note = Synthetic
EVQLVESGGGLVQPGGSLRLSCAASGFSLSSSYWISWVRQAPGKGLEWIGSIDSGSVGITYYATWVKGRFTISRDNSKNTLYLQMNSLRAEDTAVYYCARHGDNFALDLWGQGTLVTVSS

SEQ ID NO: 16 - protein, 11 aa, synthetic construct
  REGION 1..11: note = Synthetic
ARHGDNFALDL

SEQ ID NO: 17 - protein, 120 aa, synthetic construct
  REGION 1..120: note = Synthetic
EVQLVESGGGLVQPGGSLRLSCAASGFSLSSSYWISWVRQAPGKGLEWIGSIDSGSVGITYYATWVKGRFTISRDNSKNTLYLQMNSLRAEDTAVYYCARHGDNQALDLWGQGTLVTVSS

SEQ ID NO: 18 - protein, 11 aa, synthetic construct
  REGION 1..11: note = Synthetic
ARHGDNQALDL

SEQ ID NO: 19 - protein, 110 aa, synthetic construct
  REGION 1..110: note = Synthetic
DIQMTQSPSTLSASVGDRVTITCRASQSIVSWLAWYQQKPGKAPKLLIYKASTLASGVPSRFSGSGSGTEFTLTISSLQPDDFATYYCQHGYIRGDLDNVFGGGTKVEIK

SEQ ID NO: 20 - protein, 6 aa, synthetic construct
  REGION 1..6: note = Synthetic
QSIVSW

SEQ ID NO: 21 - protein, 110 aa, synthetic construct
  REGION 1..110: note = Synthetic
DIQMTQSPSTLSASVGDRVTITCRASQSIDSWLAWYQQKPGKAPKLLIYKASTLASGVPSRFSGSGSGTEFTLTISSLQPDDFATYYCQHGYIRGDLDNVFGGGTKVEIK

SEQ ID NO: 22 - protein, 6 aa, synthetic construct
  REGION 1..6: note = Synthetic
QSIDSW

SEQ ID NO: 23 - protein, 110 aa, synthetic construct
  REGION 1..110: note = Synthetic
DIQMTQSPSTLSASVGDRVTITCRASQSIESWLAWYQQKPGKAPKLLIYKASTLASGVPSRFSGSGSGTEFTLTISSLQPDDFATYYCQHGYIRGDLDNVFGGGTKVEIK

SEQ ID NO: 24 - protein, 6 aa, synthetic construct
  REGION 1..6: note = Synthetic
QSIESW

SEQ ID NO: 25 - protein, 110 aa, synthetic construct
  REGION 1..110: note = Synthetic
DIQMTQSPSTLSASVGDRVTITCRASQSIQSWLAWYQQKPGKAPKLLIYKASTLASGVPSRFSGSGSGTEFTLTISSLQPDDFATYYCQHGYIRGDLDNVFGGGTKVEIK

SEQ ID NO: 26 - protein, 6 aa, synthetic construct
  REGION 1..6: note = Synthetic
QSIQSW

SEQ ID NO: 27 - protein, 110 aa, synthetic construct
  REGION 1..110: note = Synthetic
DIQMTQSPSTLSASVGDRVTITCRASQSISSWLAWYQQKPGKAPKLLIYKASTLASGVPSRFSGSGSGTEFTLTISSLQPDDFATYYCQHGYIRGDLDNVFGGGTKVEIK

SEQ ID NO: 28 - protein, 6 aa, synthetic construct
  REGION 1..6: note = Synthetic
QSISSW

SEQ ID NO: 29 - protein, 110 aa, synthetic construct
  REGION 1..110: note = Synthetic
DIQMTQSPSTLSASVGDRVTITCRASQSITSWLAWYQQKPGKAPKLLIYKASTLASGVPSRFSGSGSGTEFTLTISSLQPDDFATYYCQHGYIRGDLDNVFGGGTKVEIK

SEQ ID NO: 30 - protein, 6 aa, synthetic construct
  REGION 1..6: note = Synthetic
QSITSW

SEQ ID NO: 31 - protein, 110 aa, synthetic construct
  REGION 1..110: note = Synthetic
DIQMTQSPSTLSASVGDRVTITCRASQSINQWLAWYQQKPGKAPKLLIYKASTLASGVPSRFSGSGSGTEFTLTISSLQPDDFATYYCQHGYIRGDLDNVFGGGTKVEIK

SEQ ID NO: 32 - protein, 6 aa, synthetic construct
  REGION 1..6: note = Synthetic
QSINQW

SEQ ID NO: 33 - protein, 110 aa, synthetic construct
  REGION 1..110: note = Synthetic
DIQMTQSPSTLSASVGDRVTITCRASQSINVWLAWYQQKPGKAPKLLIYKASTLASGVPSRFSGSGSGTEFTLTISSLQPDDFATYYCQHGYIRGDLDNVFGGGTKVEIK

SEQ ID NO: 34 - protein, 6 aa, synthetic construct
  REGION 1..6: note = Synthetic
QSINVW

SEQ ID NO: 35 - DNA, 330 bp, synthetic construct
  misc_feature 1..330: note = Synthetic
  source 1..330: mol_type = other DNA
gacatccagatgacccagtcccctagcacactgtccgccagcgtgggcgatcgtgtgaccatcacttgtcgggcttcccagagcatcagcagctggctggcttggtatcagcagaagcccggcaaggcccccaagctgctgatctacaaggcttccactttagctagcggcgtgccttctcgtttctccggatccggatccggcaccgagttcactttaaccatcagctctttacagcccgacgacttcgccacatactactgccagcacggctacatccggggcgatttagacaatgtgttcggcggcggcaccaaggtcgagatcaag

SEQ ID NO: 36 - protein, 6 aa, synthetic construct
  REGION 1..6: note = Synthetic
QSIASW

SEQ ID NO: 37 - protein, 6 aa, synthetic construct
  REGION 1..6: note = Synthetic
QSICSW

SEQ ID NO: 38 - protein, 6 aa, synthetic construct
  REGION 1..6: note = Synthetic
QSIFSW

SEQ ID NO: 39 - protein, 6 aa, synthetic construct
  REGION 1..6: note = Synthetic
QSIGSW

SEQ ID NO: 40 - protein, 6 aa, synthetic construct
  REGION 1..6: note = Synthetic
QSIHSW

SEQ ID NO: 41 - protein, 6 aa, synthetic construct
  REGION 1..6: note = Synthetic
QSIISW

SEQ ID NO: 42 - protein, 6 aa, synthetic construct
  REGION 1..6: note = Synthetic
QSIKSW

SEQ ID NO: 43 - protein, 6 aa, synthetic construct
  REGION 1..6: note = Synthetic
QSILSW

SEQ ID NO: 44 - protein, 6 aa, synthetic construct
  REGION 1..6: note = Synthetic
QSIMSW

SEQ ID NO: 45 - protein, 6 aa, synthetic construct
  REGION 1..6: note = Synthetic
QSIPSW

SEQ ID NO: 46 - protein, 6 aa, synthetic construct
  REGION 1..6: note = Synthetic
QSIRSW

SEQ ID NO: 47 - protein, 6 aa, synthetic construct
  REGION 1..6: note = Synthetic
QSIWSW

SEQ ID NO: 48 - protein, 6 aa, synthetic construct
  REGION 1..6: note = Synthetic
QSIYSW

SEQ ID NO: 49 - protein, 6 aa, synthetic construct
  REGION 1..6: note = Synthetic
QSINAW

SEQ ID NO: 50 - protein, 6 aa, synthetic construct
  REGION 1..6: note = Synthetic
QSINCW

SEQ ID NO: 51 - protein, 6 aa, synthetic construct
  REGION 1..6: note = Synthetic
QSINDW

SEQ ID NO: 52 - protein, 6 aa, synthetic construct
  REGION 1..6: note = Synthetic
QSINEW

SEQ ID NO: 53 - protein, 6 aa, synthetic construct
  REGION 1..6: note = Synthetic
QSINFW

SEQ ID NO: 54 - protein, 6 aa, synthetic construct
  REGION 1..6: note = Synthetic
QSINGW

SEQ ID NO: 55 - protein, 6 aa, synthetic construct
  REGION 1..6: note = Synthetic
QSINHW

SEQ ID NO: 56 - protein, 6 aa, synthetic construct
  REGION 1..6: note = Synthetic
QSINIW

SEQ ID NO: 57 - protein, 6 aa, synthetic construct
  REGION 1..6: note = Synthetic
QSINKW

SEQ ID NO: 58 - protein, 6 aa, synthetic construct
  REGION 1..6: note = Synthetic
QSINLW

SEQ ID NO: 59 - protein, 6 aa, synthetic construct
  REGION 1..6: note = Synthetic
QSINMW

SEQ ID NO: 60 - protein, 6 aa, synthetic construct
  REGION 1..6: note = Synthetic
QSINNW

SEQ ID NO: 61 - protein, 6 aa, synthetic construct
  REGION 1..6: note = Synthetic
QSINPW

SEQ ID NO: 62 - protein, 6 aa, synthetic construct
  REGION 1..6: note = Synthetic
QSINRW

SEQ ID NO: 63 - protein, 6 aa, synthetic construct
  REGION 1..6: note = Synthetic
QSINTW

SEQ ID NO: 64 - protein, 6 aa, synthetic construct
  REGION 1..6: note = Synthetic
QSINWW

SEQ ID NO: 65 - protein, 6 aa, synthetic construct
  REGION 1..6: note = Synthetic
QSINYW

SEQ ID NO: 66 - protein, 245 aa, synthetic construct
  REGION 1..245: note = Synthetic
EVQLVESGGGLVQPGGSLRLSCAASGFSLSSSYWISWVRQAPGKGLEWIGSIDSGSVGITYYATWVKGRFTISRDNSKNTLYLQMNSLRAEDTAVYYCARHGDNWALDLWGQGTLVTVSSGGGGSGGGGSGGGGSDIQMTQSPSTLSASVGDRVTITCRASQSISSWLAWYQQKPGKAPKLLIYKASTLASGVPSRFSGSGSGTEFTLTISSLQPDDFATYYCQHGYIRGDLDNVFGGGTKVEIK

SEQ ID NO: 67 - DNA, 813 bp, synthetic construct
  misc_feature 1..813: note = Synthetic
  source 1..813: mol_type = other DNA
accggtgccgccaccatgggctggagctgcatcatcctgttcctggtggccaccgctacaggcgtgcactctgaggtgcagctggtggagagcggaggaggactggtgcagcctggaggctccctgagactgtcttgtgccgcttctggctttagcctgagctcctcttactggatctcctgggtgcgccaggctccaggcaagggactggagtggatcggctctatcgacagcggctccgtgggcatcacctactatgccacatgggtgaagggccggttcaccatcagcagggacaactccaagaatacactgtatctgcagatgaactccctgcgggccgaggatacagccgtgtactattgcgccaggcacggcgacaattgggctctggatctgtggggacagggcaccctggtgacagtgagctccggcggcggcggctctggaggaggaggcagcggcggaggaggctccgacatccagatgacccagtctcccagcacactgtccgcttctgtgggcgatagagtgaccatcacatgtcgcgccagccagtccatctctagctggctggcttggtaccagcagaagcccggcaaggcccctaagctgctgatctataaggcctctaccctggctagcggagtgccatcccggttctctggcagcggctccggaacagagtttaccctgacaatctcctctctgcagcccgacgatttcgccacctactattgccagcacggatacatcaggggcgacctggataacgtgtttggcggcggcaccaaggtggagatcaagagatct

SEQ ID NO: 68 - protein, 245 aa, synthetic construct
  REGION 1..245: note = Synthetic
DIQMTQSPSTLSASVGDRVTITCRASQSISSWLAWYQQKPGKAPKLLIYKASTLASGVPSRFSGSGSGTEFTLTISSLQPDDFATYYCQHGYIRGDLDNVFGGGTKVEIKGGGGSGGGGSGGGGSEVQLVESGGGLVQPGGSLRLSCAASGFSLSSSYWISWVRQAPGKGLEWIGSIDSGSVGITYYATWVKGRFTISRDNSKNTLYLQMNSLRAEDTAVYYCARHGDNWALDLWGQGTLVTVSS

SEQ ID NO: 69 - DNA, 813 bp, synthetic construct
  misc_feature 1..813: note = Synthetic
  source 1..813: mol_type = other DNA
accggtgccgccaccatgggctggagctgcatcatcctgttcctggtggccaccgctacaggcgtgcactccgacatccagatgacccagtccccctctacactgagcgcctccgtgggcgatagagtgaccatcacatgtcgcgcctctcagagcatcaacagctggctggcttggtaccagcagaagcccggcaaggcccctaagctgctgatctataaggcctctaccctggctagcggagtgccatcccggttctccggctctggcagcggaacagagtttaccctgacaatcagctccctgcagcccgacgatttcgccacctactattgccagcacggatacatcaggggcgacctggataacgtgttcggcggcggcacaaaggtggagatcaagggaggaggaggcagcggcggaggaggctccggcggcggcggctctgaggtgcagctggtggagtccggaggaggactggtgcagcctggaggctctctgagactgagctgtgccgctagcggcttctccctgtctagctcctactggatctcttgggtgcgccaggctccaggcaagggactggagtggatcggcagcatcgactccggctctgtgggcatcacctactatgccacatgggtgaagggccggtttaccatctccagggacaactctaagaatacactgtatctgcagatgaactctctgcgggccgaggataccgccgtgtactattgcgccaggcacggcgacaattgggctctggatctgtggggacagggcaccctggtgacagtgtctagcagatct

SEQ ID NO: 70 - protein, 15 aa, synthetic construct
  REGION 1..15: note = Synthetic
GGGGSGGGGSGGGGS

SEQ ID NO: 71 - protein, 21 aa, synthetic construct
  REGION 1..21: note = Synthetic
IYIWAPLAGTCGVLLLSLVIT

SEQ ID NO: 72 - DNA, 63 bp, synthetic construct
  misc_feature 1..63: note = Synthetic
  source 1..63: mol_type = other DNA
atctacatctgggctccactggcaggaacctgtggcgtgctgctgctgtccctggtcatcaca

SEQ ID NO: 73 - protein, 27 aa, synthetic construct
  REGION 1..27: note = Synthetic
FWVLVVVGGVLACYSLLVTVAFIIFWV

SEQ ID NO: 74 - DNA, 81 bp, synthetic construct
  misc_feature 1..81: note = Synthetic
  source 1..81: mol_type = other DNA
ttttgggtgctggtggtggttggtggagtcctggcttgctatagcttgctagtaacagtggcctttattattttctgggtg

SEQ ID NO: 75 - protein, 41 aa, synthetic construct
  REGION 1..41: note = Synthetic
RSKRSRLLHSDYMNMTPRRPGPTRKHYQPYAPPRDFAAYRS

SEQ ID NO: 76 - DNA, 123 bp, synthetic construct
  misc_feature 1..123: note = Synthetic
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 77 - protein, 42 aa, synthetic construct
  REGION 1..42: note = Synthetic
KRGRKKLLYIFKQPFMRPVQTTQEEDGCSCRFPEEEEGGCEL

SEQ ID NO: 78 - DNA, 126 bp, synthetic construct
  misc_feature 1..126: note = Synthetic
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 79 - protein, 113 aa, synthetic construct
  REGION 1..113: note = Synthetic
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 80 - protein, 112 aa, synthetic construct
  REGION 1..112: note = Synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - DNA, 339 bp, synthetic construct
  misc_feature 1..339: note = Synthetic
  source 1..339: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 82 - RNA, 40 nt, synthetic construct
  misc_feature 1..40: note = Synthetic
  source 1..40: mol_type = other RNA
rtrcrargrgrgrtrtrcrtrgrgrartrartrcrtrgrt

SEQ ID NO: 83 - DNA, 20 bp, synthetic construct
  misc_feature 1..20: note = Synthetic
  source 1..20: mol_type = other DNA
agatgctatttcccgtataa

SEQ ID NO: 84 - DNA, 24 bp, synthetic construct
  misc_feature 1..24: note = Synthetic
  source 1..24: mol_type = other DNA
ccatagacctcatgtctagcacag

SEQ ID NO: 85 - DNA, 3261 bp, synthetic construct
  misc_feature 1..3261: note = Synthetic
  source 1..3261: mol_type = other DNA
ggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggccctgcctgtgacagccctgctgctgcctctggctctgctgctgcatgccgctagacccgatatccagatgacacagaccaccagcagcctgagcgccagcctgggcgaccgagtgactatcagctgccgggcatcccaggatatttctaagtatctgaactggtaccagcagaagcccgacggcactgtcaaactgctgatctaccacaccagtagactgcattcaggggtgcctagcaggttctccggatctggcagtgggactgactactccctgaccatctctaacctggagcaggaagatattgccacctatttctgccagcagggcaatacactgccttacacttttggcgggggaacaaagctggagatcactggcggaggaggatctggaggaggaggaagtggaggaggaggatcagaggtgaaactgcaggaaagcggaccaggactggtcgcaccttcacagagcctgtccgtgacatgtactgtctccggagtgtctctgcccgattacggcgtctcttggatccggcagccccctagaaagggactggagtggctgggcgtgatctggggaagtgaaactacctactataatagtgctctgaaatcaagactgaccatcattaaggacaactctaaaagtcaggtgtttctgaagatgaattccctgcagaccgacgatacagcaatctactattgcgccaaacactactattacggcgggagctatgccatggattactgggggcagggaacttccgtcaccgtgagcagcaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaattgttgttgttaacttgtttattgcagcttataatggttacaaataaagcaatagcatcacaaatttcacaaataaagcatttttttcactgcattctagttgtggtttgtccaaactcatcaatgtatcttagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatcttt

SEQ ID NO: 86 - DNA, 3261 bp, synthetic construct; identical to SEQ ID NO: 85
  misc_feature 1..3261: note = Synthetic
  source 1..3261: mol_type = other DNA

SEQ ID NO: 87 - DNA, 3267 bp, synthetic construct
  misc_feature 1..3267: note = Synthetic
  source 1..3267: mol_type = other DNA
ggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggccctgcctgtgacagccctgctgctgcctctggctctgctgctgcatgccgctagacccgacatccagatgacccagtccccctctacactgagcgcctccgtgggcgatagagtgaccatcacatgtcgcgcctctcagagcatcaacagctggctggcttggtaccagcagaagcccggcaaggcccctaagctgctgatctataaggcctctaccctggctagcggagtgccatcccggttctccggctctggcagcggaacagagtttaccctgacaatcagctccctgcagcccgacgatttcgccacctactattgccagcacggatacatcaggggcgacctggataacgtgttcggcggcggcacaaaggtggagatcaagggaggaggaggcagcggcggaggaggctccggcggcggcggctctgaggtgcagctggtggagtccggaggaggactggtgcagcctggaggctctctgagactgagctgtgccgctagcggcttctccctgtctagctcctactggatctcttgggtgcgccaggctccaggcaagggactggagtggatcggcagcatcgactccggctctgtgggcatcacctactatgccacatgggtgaagggccggtttaccatctccagggacaactctaagaatacactgtatctgcagatgaactctctgcgggccgaggataccgccgtgtactattgcgccaggcacggcgacaattgggctctggatctgtggggacagggcaccctggtgacagtgtctagcaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaattgttgttgttaacttgtttattgcagcttataatggttacaaataaagcaatagcatcacaaatttcacaaataaagcatttttttcactgcattctagttgtggtttgtccaaactcatcaatgtatcttagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatcttt

SEQ ID NO: 88 - DNA, 3270 bp, synthetic construct
  misc_feature 1..3270: note = Synthetic
  source 1..3270: mol_type = other DNA
ggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggccctgcctgtgacagccctgctgctgcctctggctctgctgctgcatgccgctagacccgacatccagatgacccagtccccctctacactgagcgcctccgtgggcgatagagtgaccatcacatgtcgcgcctctcagagcatcaacagctggctggcttggtaccagcagaagcccggcaaggcccctaagctgctgatctataaggcctctaccctggctagcggagtgccatcccggttctccggctctggcagcggaacagagtttaccctgacaatcagctccctgcagcccgacgatttcgccacctactattgccagcacggatacatcaggggcgacctggataacgtgttcggcggcggcacaaaggtggagatcaagggaggaggaggcagcggcggaggaggctccggcggcggcggctctgaggtgcagctggtggagtccggaggaggactggtgcagcctggaggctctctgagactgagctgtgccgctagcggcttctccctgtctagctcctactggatctcttgggtgcgccaggctccaggcaagggactggagtggatcggcagcatcgactccggctctgtgggcatcacctactatgccacatgggtgaagggccggtttaccatctccagggacaactctaagaatacactgtatctgcagatgaactctctgcgggccgaggataccgccgtgtactattgcgccaggcacggcgacaattgggctctggatctgtggggacagggcaccctggtgacagtgtctagcaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaattgttgttgttaacttgtttattgcagcttataatggttacaaataaagcaatagcatcacaaatttcacaaataaagcatttttttcactgcattctagttgtggtttgtccaaactcatcaatgtatcttagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatcttt

SEQ ID NO: 89 - protein, 130 aa, synthetic construct
  REGION 1..130: note = Synthetic
LEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRA

SEQ ID NO: 90 - protein, 95 aa, synthetic construct
  REGION 1..95: note = Synthetic
PDIQNPDPAVYQLRDSKSSDKSVCLFTDFDSQTQVSQSKDSDVYITDKCVLDMRSMDFKSNSAVAWSQKSDFACANAFQNSIIPEDTFFPSPESS

SEQ ID NO: 91 - protein, 448 aa, synthetic construct
  REGION 1..448: note = Synthetic
QVQLVQSGAEVKKPGSSVKVSCKASGFAFTDYYIHWVRQAPGQGLEWMGWISPGNVNTKYNENFKGRVTITADKSTSTAYMELSSLRSEDTAVYYCARDGYSLYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 92 - protein, 474 aa, synthetic construct
  REGION 1..474: note = Synthetic
QVQLVQSGAEVKKPGSSVKVSCKASGFAFTDYYIHWVRQAPGQGLEWMGWISPGNVNTKYNENFKGRVTITADKSTSTAYMELSSLRSEDTAVYYCARDGYSLYYFDYWGQGTLVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRASDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 93 - protein, 474 aa, synthetic construct
  REGION 1..474: note = Synthetic
EVQLVQSGAEVKKPGSSVKVSCKASGFAFTDYYIHWVRQAPGQGLEWMGWISPGNVNTKYNENFKGRVTITADKSTSTAYMELSSLRSEDTAVYYCARDGYSLYYFDYWGQGTLVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRASDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 94 - protein, 449 aa, synthetic construct
  REGION 1..449: note = Synthetic
EVQLVESGGGLVQPGGSLRLSCAASGFSLSSSYWISWVRQAPGKGLEWIGSIDSGSVGITYYATWVKGRFTISRDNSKNTLYLQMNSLRAEDTAVYYCARHGDNWALDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 95 - protein, 682 aa, synthetic construct
  REGION 1..682: note = Synthetic
EVQLVESGGGLVQPGGSLRLSCAASGFSLSSSYWISWVRQAPGKGLEWIGSIDSGSVGITYYATWVKGRFTISRDNSKNTLYLQMNSLRAEDTAVYYCARHGDNWALDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSEVQLVESGGGLVQPGGSLRLSCAASGFSLSSSYWISWVRQAPGKGLEWIGSIDSGSVGITYYATWVKGRFTISRDNSKNTLYLQMNSLRAEDTAVYYCARHGDNWALDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 96 - protein, 475 aa, synthetic construct
  REGION 1..475: note = Synthetic
EVQLVESGGGLVQPGGSLRLSCAASGFSLSSSYWISWVRQAPGKGLEWIGSIDSGSVGITYYATWVKGRFTISRDNSKNTLYLQMNSLRAEDTAVYYCARHGDNWALDLWGQGTLVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRASDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 97 - protein, 732 aa, synthetic construct
  REGION 1..732: note = Synthetic
EVQLVESGGGLVQPGGSLRLSCAASGFSLSSSYWISWVRQAPGKGLEWIGSIDSGSVGITYYATWVKGRFTISRDNSKNTLYLQMNSLRAEDTAVYYCARHGDNWALDLWGQGTLVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRGGGGSGGGGSEVQLVESGGGLVQPGGSLRLSCAASGFSLSSSYWISWVRQAPGKGLEWIGSIDSGSVGITYYATWVKGRFTISRDNSKNTLYLQMNSLRAEDTAVYYCARHGDNWALDLWGQGTLVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 98 - protein, 705 aa, synthetic construct
  REGION 1..705: note = Synthetic
EVQLVESGGGLVQPGGSLRLSCAASGFSLSSSYWISWVRQAPGKGLEWIGSIDSGSVGITYYATWVKGRFTISRDNSKNTLYLQMNSLRAEDTAVYYCARHGDNWALDLWGQGTLVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRGGGGSGGGGSQVQLVQSGAEVKKPGSSVKVSCKASGFAFTDYYIHWVRQAPGQGLEWMGWISPGNVNTKYNENFKGRVTITADKSTSTAYMELSSLRSEDTAVYYCARDGYSLYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 99 - protein, 707 aa, synthetic construct
  REGION 1..707: note = Synthetic
EVQLVESGGGLVQPGGSLRLSCAASGFSLSSSYWISWVRQAPGKGLEWIGSIDSGSVGITYYATWVKGRFTISRDNSKNTLYLQMNSLRAEDTAVYYCARHGDNWALDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSQVQLVQSGAEVKKPGSSVKVSCKASGFAFTDYYIHWVRQAPGQGLEWMGWISPGNVNTKYNENFKGRVTITADKSTSTAYMELSSLRSEDTAVYYCARDGYSLYYFDYWGQGTLVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRASDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 100 - protein, 219 aa, synthetic construct
  REGION 1..219: note = Synthetic
DIVMTQSPDSLAVSLGERATINCKSSQSLLNSRTRKNYLAWYQQKPGQPPKLLIYWASTRQSGVPDRFSGSGSGTDFTLTISSLQAEDVAVYYCTQSHTLRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 207 aa, synthetic construct
  REGION 1..207: note = Synthetic
DIVMTQSPDSLAVSLGERATINCKSSQSLLNSRTRKNYLAWYQQKPGQPPKLLIYWASTRQSGVPDRFSGSGSGTDFTLTISSLQAEDVAVYYCTQSHTLRTFGGGTKVEIKPDIQNPDPAVYQLRDSKSSDKSVCLFTDFDSQTQVSQSKDSDVYITDKCVLDMRSMDFKSNSAVAWSQKSDFACANAFQNSIIPEDTFFPSPESS

SEQ ID NO: 102 - protein, 207 aa, synthetic construct
  REGION 1..207: note = Synthetic
DIVMTQSPDSLAVSLGERATINCKSSQSLLNSRTRKNYLAWYQQKPGQPPKLLIYWASTRQSGVPDRFSGSGSGTDFTLTISSLQAEDVAVYYCTQSHTLRTFGGGTKVEIKPDIQNPDPAVYQLRDSKSSDKSVCLFTDFDSQTQVSQSKDSDVYITDKCVLDMRSMDFKSNSAVAWSQKSDFACANAFQNSIIPEDTFFPAPESS

SEQ ID NO: 103 - protein, 217 aa, synthetic construct
  REGION 1..217: note = Synthetic
DIQMTQSPSTLSASVGDRVTITCRASQSISSWLAWYQQKPGKAPKLLIYKASTLASGVPSRFSGSGSGTEFTLTISSLQPDDFATYYCQHGYIRGDLDNVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 205 aa, synthetic construct
  REGION 1..205: note = Synthetic
DIQMTQSPSTLSASVGDRVTITCRASQSISSWLAWYQQKPGKAPKLLIYKASTLASGVPSRFSGSGSGTEFTLTISSLQPDDFATYYCQHGYIRGDLDNVFGGGTKVEIKPDIQNPDPAVYQLRDSKSSDKSVCLFTDFDSQTQVSQSKDSDVYITDKCVLDMRSMDFKSNSAVAWSQKSDFACANAFQNSIIPEDTFFPSPESS

SEQ ID NO: 105 - protein, 205 aa, synthetic construct
  REGION 1..205: note = Synthetic
DIQMTQSPSTLSASVGDRVTITCRASQSISSWLAWYQQKPGKAPKLLIYKASTLASGVPSRFSGSGSGTEFTLTISSLQPDDFATYYCQHGYIRGDLDNVFGGGTKVEIKPDIQNPDPAVYQLRDSKSSDKSVCLFTDFDSQTQVSQSKDSDVYITDKCVLDMRSMDFKSNSAVAWSQKSDFACANAFQNSIIPEDTFFPAPESS

SEQ ID NO: 106 - protein, 449 aa, synthetic construct
  REGION 1..449: note = Synthetic
EVQLVESGGGLVQPGGSLRLSCAASGFSLSSSYWISWVRQAPGKGLEWIGSIDSGSVGITYYATWVKGRFTISRDNSKNTLYLQMNSLRAEDTAVYYCARHGDNWALDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 107 - protein, 226 aa, synthetic construct
  REGION 1..226: note = Synthetic
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 108 - DNA, 1407 bp, synthetic construct
  misc_feature 1..1407: note = Synthetic
  source 1..1407: mol_type = other DNA
atggagtttgggctgagctggctttttcttgtcgcgattcttaagggtgtacagtgtcaggtgcagcttgtgcagtctggggcagaagtgaagaagcctgggtctagtgtcaaggtgtcatgcaaggctagcgggttcgcctttactgactactacatccactgggtgcggcaggctcccggacaagggttggagtggatgggatggatctccccaggcaatgtcaacacaaagtacaacgagaacttcaaaggccgcgtcaccattaccgccgacaagagcacctccacagcctacatggagctgtccagcctcagaagcgaggacactgccgtctactactgtgccagggatgggtactccctgtattactttgattactggggccagggcacactggtgacagtgagctccgcgtcgaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatgccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcttaagcctgtctccgggttaatag

SEQ ID NO: 109 - DNA, 1422 bp, synthetic construct
  misc_feature 1..1422: note = Synthetic
  source 1..1422: mol_type = other DNA
caggtgcagcttgtgcagtctggggcagaagtgaagaagcctgggtctagtgtcaaggtgtcatgcaaggctagcgggttcgcctttactgactactacatccactgggtgcggcaggctcccggacaagggttggagtggatgggatggatctccccaggcaatgtcaacacaaagtacaacgagaacttcaaaggccgcgtcaccattaccgccgacaagagcacctccacagcctacatggagctgtccagcctcagaagcgaggacactgccgtctactactgtgccagggatgggtactccctgtattactttgattactggggccagggcacactggtgacagtgctggaggacctgaagaacgtgttccctcccgaggtggccgtgttcgaacccagcgaggccgagatcagccacacccagaaggccaccctggtgtgtctggccaccggcttctaccccgaccacgtggagctgagctggtgggtgaacggcaaggaggtgcacagcggcgtgtgtaccgaccctcagcccctgaaggagcagcccgccctgcaggacagcaggtacgccctgagcagcaggctgagagtgagcgccaccttctggcagaaccccaggaaccacttcaggtgccaggtgcagttctacggcctgagcgagaacgacgagtggacccaggacagggccaagcccgtgacccagatcgtgagcgctgaggcctggggcagagcctctgacaaaactcacacatgcccaccgtgtccagcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatgccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcttaagcctgtctccgggt

SEQ ID NO: 110 - DNA, 1485 bp, synthetic construct
  misc_feature 1..1485: note = Synthetic
  source 1..1485: mol_type = other DNA
atggagtttgggctgagctggctttttcttgtcgcgattcttaagggtgtacagtgtgaggtgcagcttgtgcagtctggggcagaagtgaagaagcctgggtctagtgtcaaggtgtcatgcaaggctagcgggttcgcctttactgactactacatccactgggtgcggcaggctcccggacaagggttggagtggatgggatggatctccccaggcaatgtcaacacaaagtacaacgagaacttcaaaggccgcgtcaccattaccgccgacaagagcacctccacagcctacatggagctgtccagcctcagaagcgaggacactgccgtctactactgtgccagggatgggtactccctgtattactttgattactggggccagggcacactggtgacagtgctggaggacctgaagaacgtgttccctcccgaggtggccgtgttcgaacccagcgaggccgagatcagccacacccagaaggccaccctggtgtgtctggccaccggcttctaccccgaccacgtggagctgagctggtgggtgaacggcaaggaggtgcacagcggcgtgtgtaccgaccctcagcccctgaaggagcagcccgccctgcaggacagcaggtacgccctgagcagcaggctgagagtgagcgccaccttctggcagaaccccaggaaccacttcaggtgccaggtgcagttctacggcctgagcgagaacgacgagtggacccaggacagggccaagcccgtgacccagatcgtgagcgctgaggcctggggcagagcctctgacaaaactcacacatgcccaccgtgtccagcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatgccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcttaagcctgtctccgggttaatag

SEQ ID NO: 111 - DNA, 1410 bp, synthetic construct
  misc_feature 1..1410: note = Synthetic
  source 1..1410: mol_type = other DNA
atggagtttgggctgagctggctttttcttgtcgcgattcttaagggtgtacagtgtgaggtgcagctcgtcgagtccggaggaggtttagtccaacccggtggatctttaaggctgtcttgtgctgcttccggcttttctttatcctccagctactggatctcttgggtgcggcaagctcccggaaagggactcgagtggatcggctccatcgattccggatccgtgggcattacctactacgccacatgggtgaaaggtcgtttcacaatctcccgggacaactccaagaacaccctctatttacagatgaactctttaagggccgaggataccgctgtgtactactgtgcccggcacggcgacaattgggctttagatctgtggggccaaggtacactggtgacagtgagcagcgcgtcgaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctgcaccctgcccccatcccgggaggagatgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgttagcaagctcaccgtggacaagagcaggtggcagcaggggaacgtcttctcatgctccgtgatgcatgaggctctgcacaaccactacacgcagaagagcttaagcctgtctccgggttaatag

SEQ ID NO: 112 - DNA, 2046 bp, synthetic construct
  misc_feature 1..2046: note = Synthetic
  source 1..2046: mol_type = other DNA
gaggtgcagctcgtcgagtccggaggaggtttagtccaacccggtggatctttaaggctgtcttgtgctgcttccggcttttctttatcctccagctactggatctcttgggtgcggcaagctcccggaaagggactcgagtggatcggctccatcgattccggatccgtgggcattacctactacgccacatgggtgaaaggtcgtttcacaatctcccgggacaactccaagaacaccctctatttacagatgaactctttaagggccgaggataccgctgtgtactactgtgcccggcacggcgacaattgggctttagatctgtggggccaaggtacactggtgacagtgagcagcgcgtcgaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtggtggaggtggcagtggaggcggaggatctgaggtgcagctcgtcgagtccggaggaggtttagtccaacccggtggatctttaaggctgtcttgtgctgcttccggcttttctttatcctccagctactggatctcttgggtgcggcaagctcccggaaagggactcgagtggatcggctccatcgattccggatccgtgggcattacctactacgccacatgggtgaaaggtcgtttcacaatctcccgggacaactccaagaacaccctctatttacagatgaactctttaagggccgaggataccgctgtgtactactgtgcccggcacggcgacaattgggctttagatctgtggggccaaggtacactggtgacagtgagcagcgcgtcgaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctgcaccctgcccccatcccgggaggagatgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgttagcaagctcaccgtggacaagagcaggtggcagcaggggaacgtcttctcatgctccgtgatgcatgaggctctgcacaaccactacacgcagaagagcttaagcctgtctccgggt

SEQ ID NO: 113 - DNA, 1488 bp, synthetic construct
  misc_feature 1..1488: note = Synthetic
  source 1..1488: mol_type = other DNA
atggagtttgggctgagctggctttttcttgtcgcgattcttaagggtgtacagtgtgaggtgcagctcgtcgagtccggaggaggtttagtccaacccggtggatctttaaggctgtcttgtgctgcttccggcttttctttatcctccagctactggatctcttgggtgcggcaagctcccggaaagggactcgagtggatcggctccatcgattccggatccgtgggcattacctactacgccacatgggtgaaaggtcgtttcacaatctcccgggacaactccaagaacaccctctatttacagatgaactctttaagggccgaggataccgctgtgtactactgtgcccggcacggcgacaattgggctttagatctgtggggccaaggtacactggtgacagtgctggaggacctgaagaacgtgttccctcccgaggtggccgtgttcgaacccagcgaggccgagatcagccacacccagaaggccaccctggtgtgtctggccaccggcttctaccccgaccacgtggagctgagctggtgggtgaacggcaaggaggtgcacagcggcgtgtgtaccgaccctcagcccctgaaggagcagcccgccctgcaggacagcaggtacgccctgagcagcaggctgagagtgagcgccaccttctggcagaaccccaggaaccacttcaggtgccaggtgcagttctacggcctgagcgagaacgacgagtggacccaggacagggccaagcccgtgacccagatcgtgagcgctgaggcctggggcagagcctctgacaaaactcacacatgcccaccgtgcccagcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctgcaccctgcccccatcccgggaggagatgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgttagcaagctcaccgtggacaagagcaggtggcagcaggggaacgtcttctcatgctccgtgatgcatgaggctctgcacaaccactacacgcagaagagcttaagcctgtctccgggttaatag

SEQ ID NO: 114 - DNA, 2196 bp, synthetic construct
  misc_feature 1..2196: note = Synthetic
  source 1..2196: mol_type = other DNA
gaggtgcagctcgtcgagtccggaggaggtttagtccaacccggtggatctttaaggctgtcttgtgctgcttccggcttttctttatcctccagctactggatctcttgggtgcggcaagctcccggaaagggactcgagtggatcggctccatcgattccggatccgtgggcattacctactacgccacatgggtgaaaggtcgtttcacaatctcccgggacaactccaagaacaccctctatttacagatgaactctttaagggccgaggataccgctgtgtactactgtgcccggcacggcgacaattgggctttagatctgtggggccaaggtacactggtgacagtgctggaggacctgaagaacgtgttccctcccgaggtggccgtgttcgaacccagcgaggccgagatcagccacacccagaaggccaccctggtgtgtctggccaccggcttctaccccgaccacgtggagctgagctggtgggtgaacggcaaggaggtgcacagcggcgtgtgtaccgaccctcagcccctgaaggagcagcccgccctgcaggacagcaggtacgccctgagcagcaggctgagagtgagcgccaccttctggcagaaccccaggaaccacttcaggtgccaggtgcagttctacggcctgagcgagaacgacgagtggacccaggacagggccaagcccgtgacccagatcgtgagcgctgaggcctggggcagaggtggaggtggcagtggaggcggaggatctgaggtgcagctcgtcgagtccggaggaggtttagtccaacccggtggatctttaaggctgtcttgtgctgcttccggcttttctttatcctccagctactggatctcttgggtgcggcaagctcccggaaagggactcgagtggatcggctccatcgattccggatccgtgggcattacctactacgccacatgggtgaaaggtcgtttcacaatctcccgggacaactccaagaacaccctctatttacagatgaactctttaagggccgaggataccgctgtgtactactgtgcccggcacggcgacaattgggctttagatctgtggggccaaggtacactggtgacagtgctggaggacctgaagaacgtgttccctcccgaggtggccgtgttcgaacccagcgaggccgagatcagccacacccagaaggccaccctggtgtgtctggccaccggcttctaccccgaccacgtggagctgagctggtgggtgaacggcaaggaggtgcacagcggcgtgtgtaccgaccctcagcccctgaaggagcagcccgccctgcaggacagcaggtacgccctgagcagcaggctgagagtgagcgccaccttctggcagaaccccaggaaccacttcaggtgccaggtgcagttctacggcctgagcgagaacgacgagtggacccaggacagggccaagcccgtgacccagatcgtgagcgctgaggcctggggcagagcctctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctgcaccctgcccccatcccgggaggagatgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgttagcaagctcaccgtggacaagagcaggtggcagcaggggaacgtcttctcatgctccgtgatgcatgaggctctgcacaaccactacacgcagaagagcttaagcctgtctccgggt

SEQ ID NO: 115 - DNA, 2178 bp, synthetic construct
  misc_feature 1..2178: note = Synthetic
  source 1..2178: mol_type = other DNA
atggagtttgggctgagctggctttttcttgtcgcgattcttaagggtgtacagtgtgaggtgcagctcgtcgagtccggaggaggtttagtccaacccggtggatctttaaggctgtcttgtgctgcttccggcttttctttatcctccagctactggatctcttgggtgcggcaagctcccggaaagggactcgagtggatcggctccatcgattccggatccgtgggcattacctactacgccacatgggtgaaaggtcgtttcacaatctcccgggacaactccaagaacaccctctatttacagatgaactctttaagggccgaggataccgctgtgtactactgtgcccggcacggcgacaattgggctttagatctgtggggccaaggtacactggtgacagtgctggaggacctgaagaacgtgttccctcccgaggtggccgtgttcgaacccagcgaggccgagatcagccacacccagaaggccaccctggtgtgtctggccaccggcttctaccccgaccacgtggagctgagctggtgggtgaacggcaaggaggtgcacagcggcgtgtgtaccgaccctcagcccctgaaggagcagcccgccctgcaggacagcaggtacgccctgagcagcaggctgagagtgagcgccaccttctggcagaaccccaggaaccacttcaggtgccaggtgcagttctacggcctgagcgagaacgacgagtggacccaggacagggccaagcccgtgacccagatcgtgagcgcggaggcctggggcagaggtggaggtggcagtggaggcggaggatctcaggtgcagcttgtgcagtctggggcagaagtgaagaagcctgggtctagtgtcaaggtgtcatgcaaggctagcgggttcgcctttactgactactacatccactgggtgcggcaggctcccggacaagggttggagtggatgggatggatctccccaggcaatgtcaacacaaagtacaacgagaacttcaaaggccgcgtcaccattaccgccgacaagagcacctccacagcctacatggagctgtccagcctcagaagcgaggacactgccgtctactactgtgccagggatgggtactccctgtattactttgattactggggccagggcacactggtgacagtgagctccgcgtcgaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatgccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcttaagcctgtctccgggttaatag

SEQ ID NO: 116 - DNA, 2121 bp, synthetic construct
  misc_feature 1..2121: note = Synthetic
  source 1..2121: mol_type = other DNA
gaggtgcagctcgtcgagtccggaggaggtttagtccaacccggtggatctttaaggctgtcttgtgctgcttccggcttttctttatcctccagctactggatctcttgggtgcggcaagctcccggaaagggactcgagtggatcggctccatcgattccggatccgtgggcattacctactacgccacatgggtgaaaggtcgtttcacaatctcccgggacaactccaagaacaccctctatttacagatgaactctttaagggccgaggataccgctgtgtactactgtgcccggcacggcgacaattgggctttagatctgtggggccaaggtacactggtgacagtgagcagcgcgtcgaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtggtggaggtggcagtggaggcggaggatctcaggtgcagcttgtgcagtctggggcagaagtgaagaagcctgggtctagtgtcaaggtgtcatgcaaggctagcgggttcgcctttactgactactacatccactgggtgcggcaggctcccggacaagggttggagtggatgggatggatctccccaggcaatgtcaacacaaagtacaacgagaacttcaaaggccgcgtcaccattaccgccgacaagagcacctccacagcctacatggagctgtccagcctcagaagcgaggacactgccgtctactactgtgccagggatgggtactccctgtattactttgattactggggccagggcacactggtgacagtgctggaggacctgaagaacgtgttccctcccgaggtggccgtgttcgaacccagcgaggccgagatcagccacacccagaaggccaccctggtgtgtctggccaccggcttctaccccgaccacgtggagctgagctggtgggtgaacggcaaggaggtgcacagcggcgtgtgtaccgaccctcagcccctgaaggagcagcccgccctgcaggacagcaggtacgccctgagcagcaggctgagagtgagcgccaccttctggcagaaccccaggaaccacttcaggtgccaggtgcagttctacggcctgagcgagaacgacgagtggacccaggacagggccaagcccgtgacccagatcgtgagcgctgaggcctggggcagagcctctgacaaaactcacacatgcccaccgtgtccagcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatgccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcttaagcctgtctccgggt

SEQ ID NO: 117 - DNA, 729 bp, synthetic construct
  misc_feature 1..729: note = Synthetic
  source 1..729: mol_type = other DNA
atggacatgcgcgtgcccgcccagctgctgggcctgctgctgctgtggttccccggatcccgatgcgatatcgtgatgacccagagcccagactcccttgctgtctccctcggcgaaagagcaaccatcaactgcaagagctcccaaagcctgctgaactccaggaccaggaagaattacctggcctggtatcagcagaagcccggccagcctcctaagctgctcatctactgggcctccacccggcagtctggggtgcccgatcggtttagtggatctgggagcgggacagacttcacattgacaattagctcactgcaggccgaggacgtggccgtctactactgtactcagagccacactctccgcacattcggcggagggactaaagtggagattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaatag

SEQ ID NO: 118 - DNA, 621 bp, synthetic construct
  misc_feature 1..621: note = Synthetic
  source 1..621: mol_type = other DNA
gatatcgtgatgacccagagcccagactcccttgctgtctccctcggcgaaagagcaaccatcaactgcaagagctcccaaagcctgctgaactccaggaccaggaagaattacctggcctggtatcagcagaagcccggccagcctcctaagctgctcatctactgggcctccacccggcagtctggggtgcccgatcggtttagtggatctgggagcgggacagacttcacattgacaattagctcactgcaggccgaggacgtggccgtctactactgtactcagagccacactctccgcacattcggcggagggactaaagtggagattaagcccgacatccagaaccccgaccccgccgtgtaccagctgagagacagcaagagcagcgacaagagcgtgtgcctgttcaccgacttcgacagccagacccaggtgagccagagcaaggactccgacgtgtatatcaccgacaagtgcgtgctggacatgaggagcatggacttcaagagcaacagcgccgtggcctggagccagaagagcgacttcgcctgcgccaacgccttccagaacagcatcatccccgaggacaccttcttccccagccccgagagcagc

SEQ ID NO: 119 - DNA, 693 bp, synthetic construct
  misc_feature 1..693: note = Synthetic
  source 1..693: mol_type = other DNA
atggacatgcgcgtgcccgcccagctgctgggcctgctgctgctgtggttccccggatcccgatgcgatatcgtgatgacccagagcccagactcccttgctgtctccctcggcgaaagagcaaccatcaactgcaagagctcccaaagcctgctgaactccaggaccaggaagaattacctggcctggtatcagcagaagcccggccagcctcctaagctgctcatctactgggcctccacccggcagtctggggtgcccgatcggtttagtggatctgggagcgggacagacttcacattgacaattagctcactgcaggccgaggacgtggccgtctactactgtactcagagccacactctccgcacattcggcggagggactaaagtggagattaagcccgacatccagaaccccgaccccgccgtgtaccagctgagagacagcaagagcagcgacaagagcgtgtgcctgttcaccgacttcgacagccagacccaggtgagccagagcaaggactccgacgtgtatatcaccgacaagtgcgtgctggacatgaggagcatggacttcaagagcaacagcgccgtggcctggagccagaagagcgacttcgcctgcgccaacgccttccagaacagcatcatccccgaggacaccttcttccccgcccccgagagcagctaatag

SEQ ID NO: 120 - DNA, 723 bp, synthetic construct
  misc_feature 1..723: note = Synthetic
  source 1..723: mol_type = other DNA
atggacatgcgcgtgcccgcccagctgctgggcctgctgctgctgtggttccccggatcccgatgcgacatccagatgacccagtcccctagcacactgtccgccagcgtgggcgatcgtgtgaccatcacttgtcgggcttcccagagcatcagcagctggctggcttggtatcagcagaagcccggcaaggcccccaagctgctgatctacaaggcttccactttagctagcggcgtgccttctcgtttctccggatccggatccggcaccgagttcactttaaccatcagctctttacagcccgacgacttcgccacatactactgccagcacggctacatccggggcgatttagacaatgtgttcggcggcggcaccaaggtcgagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaatag

SEQ ID NO: 121 - DNA, 615 bp, synthetic construct
  misc_feature 1..615: note = Synthetic
  source 1..615: mol_type = other DNA
gacatccagatgacccagtcccctagcacactgtccgccagcgtgggcgatcgtgtgaccatcacttgtcgggcttcccagagcatcagcagctggctggcttggtatcagcagaagcccggcaaggcccccaagctgctgatctacaaggcttccactttagctagcggcgtgccttctcgtttctccggatccggatccggcaccgagttcactttaaccatcagctctttacagcccgacgacttcgccacatactactgccagcacggctacatccggggcgatttagacaatgtgttcggcggcggcaccaaggtcgagatcaagcccgacatccagaaccccgaccccgccgtgtaccagctgagagacagcaagagcagcgacaagagcgtgtgcctgttcaccgacttcgacagccagacccaggtgagccagagcaaggactccgacgtgtatatcaccgacaagtgcgtgctggacatgaggagcatggacttcaagagcaacagcgccgtggcctggagccagaagagcgacttcgcctgcgccaacgccttccagaacagcatcatccccgaggacaccttcttccccagccccgagagcagc

SEQ ID NO: 122 - DNA, 687 bp, synthetic construct
  misc_feature 1..687: note = Synthetic
  source 1..687: mol_type = other DNA
atggacatgcgcgtgcccgcccagctgctgggcctgctgctgctgtggttccccggatcccgatgcgacatccagatgacccagtcccctagcacactgtccgccagcgtgggcgatcgtgtgaccatcacttgtcgggcttcccagagcatcagcagctggctggcttggtatcagcagaagcccggcaaggcccccaagctgctgatctacaaggcttccactttagctagcggcgtgccttctcgtttctccggatccggatccggcaccgagttcactttaaccatcagctctttacagcccgacgacttcgccacatactactgccagcacggctacatccggggcgatttagacaatgtgttcggcggcggcaccaaggtcgagatcaagcccgacatccagaaccccgaccccgccgtgtaccagctgagagacagcaagagcagcgacaagagcgtgtgcctgttcaccgacttcgacagccagacccaggtgagccagagcaaggactccgacgtgtatatcaccgacaagtgcgtgctggacatgaggagcatggacttcaagagcaacagcgccgtggcctggagccagaagagcgacttcgcctgcgccaacgccttccagaacagcatcatccccgaggacaccttcttccccgcccccgagagcagctaatag

SEQ ID NO: 123 - DNA, 1410 bp, synthetic construct
  misc_feature 1..1410: note = Synthetic
  source 1..1410: mol_type = other DNA
atggagtttgggctgagctggctttttcttgtcgcgattcttaagggtgtacagtgtgaggtgcagctcgtcgagtccggaggaggtttagtccaacccggtggatctttaaggctgtcttgtgctgcttccggcttttctttatcctccagctactggatctcttgggtgcggcaagctcccggaaagggactcgagtggatcggctccatcgattccggatccgtgggcattacctactacgccacatgggtgaaaggtcgtttcacaatctcccgggacaactccaagaacaccctctatttacagatgaactctttaagggccgaggataccgctgtgtactactgtgcccggcacggcgacaattgggctttagatctgtggggccaaggtacactggtgacagtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggccgtcctacagtcctcaggactctactccctcagcagcgtggtgaccgtgccctccagcagcttgggcacccagacctacatctgcaacgtgaatcacaagcccagcaacaccaaggtggacaagcgggttgagcccaaatcttgtgacaaaactcacacatgcccaccgtgcccagcacctgaagccgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttgatga

SEQ ID NO: 124 - DNA, 756 bp, synthetic construct
  misc_feature 1..756: note = Synthetic
  source 1..756: mol_type = other DNA
atggtgctcgccgcccctctcctgctcggatttctcctgctggctctcgaactgagacccaggggagaggccgacaaaactcacacatgcccaccgtgcccagcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctgcaccctgcccccatcccgggaggagatgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgttagcaagctcaccgtggacaagagcaggtggcagcaggggaacgtcttctcatgctccgtgatgcatgaggctctgcacaaccactacacgcagaagagcttaagcctgtctccgggttaatag